(19) United States
(12) Patent Application Publication
(10) Pub. No.: US 2022/0403367 A1
(43) Pub. Date: Dec. 22, 2022

(54) MICROBIOME ENGINEERING THROUGH ENGINEERED MOBILE GENETIC ELEMENTS

(71) Applicant: THE TRUSTEES OF COLUMBIA UNIVERSITY IN THE CITY OF NEW YORK, NEW YORK, NY (US)

(72) Inventor: Harris He WANG, New York, NY (US)

(73) Assignee: THE TRUSTEES OF COLUMBIA UNIVERSITY IN THE CITY OF NEW YORK, NEW YORK, NY (US)

(21) Appl. No.: 17/422,373

(22) PCT Filed: Jan. 13, 2020
(86) PCT No.: PCT/US20/13368

§ 371 Date: Jul. 12, 2021

Related U.S. Application Data
(60) Provisional application No. 62/791,508, filed on Jan. 11, 2019.
(60) Provisional application No. 62/813,504, filed on Mar. 4, 2019.

(51) Int. Cl. C12N 15/10; C12N 1/20; A61K 35/74

(52) U.S. Cl. CPC C12N 15/102; C12N 15/1086; C12N 1/20; A61K 35/74

(57) ABSTRACT

The present invention relates to utilizing engineered horizontal gene transfer elements and high-throughput selection strategies to tag and retrieve genetically modified native commensal strains from the mammalian gut. In certain aspects, the present invention relates to methods wherein isolated bacteria from the mammalian gut microbiome that were amenable to genetic manipulation were redeployed back into the mammalian subject as host-optimized engineerable probiotics.

### STATEMENT REGARDING FEDERALLY SPONSORED RESEARCH OR DEVELOPMENT

This invention was made with government support under OD009172 and GM110714 awarded by the National Institutes of Health, 1453219 awarded by the National Science Foundation, W911NF-15-2-0065 awarded by Army/ARO and N00014-15-1-2704 awarded by Navy/ONR. The government has certain rights in the invention.

### SEQUENCE LISTING STATEMENT

The text of the computer readable sequence listing filed herewith, titled “38839-252_SEQUENCE_LISTING”, created Jul. 12, 2021, having a file size of 33,514 bytes, is hereby incorporated by reference in its entirety.

### BACKGROUND

Microbes in nature live in open, dynamic and challenging habitats that are difficult, if not impossible, to replicate in a laboratory setting (Stewart, E. J. Growing unculturable bacteria. Journal of Bacteriology 194, 4151-4160 (2012)). They form complex communities whose physiology and metabolism are interlinked in ways that have yet to be fully elucidated (Little, A. E., Robinson, C. J., Peterson, S. B., Raffa, K. F. & Handelsman, J. Rules of Engagement: Interspecies Interactions that Regulate Microbial Communities. Annu Rev Microbiol 62, 375-401 (2008)). As a result, far fewer microbes have been cultivated in the laboratory, in stark contrast to their large diversity in the wild (Alain, K. & Querellou, J. Cultivating the uncultured: Limits, advances and future challenges. Extremophiles 13, 583-594 (2009)). Even fewer of these domesticated microbes are genetically tractable, such that we can add, delete or modify their genetic content. Recent advances to survey microbial populations by deep sequencing have greatly outpaced genetic methods to manipulate them. For example, of the thousands of bacterial species from the mammalian gut that have been isolated and sequenced, only a handful of them are genetically modifiable (Yaung, S. J., Church, G. M. & Wang, H. H. Recent progress in engineering human-associated microbiomes. Methods Mol. Biol. 1151, 3-25 (2014), Cuív, P. Ó. et al. Isolation of genetically tractable most-wanted bacteria by metaparental mating. Sci. Rep. 5, 13282 (2015)). The inability to genetically alter a bacterium hinders our basic understanding of the organism and has stalled efforts to mechanistically connect the gut microbiome with host physiology, despite a plethora of correlative evidence (Blaser, M., Bork, P., Fraser, C., Knight, R. & Wang, J. The microbiome explored: recent insights and future challenges. Nat. Rev. Microbiol. 11, 213-7 (2013)). Furthermore, the recalcitrance of these intractable microbes to genome and metabolic engineering limits their bio-industrial and probiotic potential.

Despite collective efforts to mend this gap, many microbes remain genetically intractable due to inherent biological factors, such as the restriction and methylation and CRISPR-Cas systems (Thomas, C. M. & Nielsen, K. M. Mechanisms of, and barriers to, horizontal gene transfer between bacteria. Nat. Rev. Microbiol. 3, 711-721 (2005), Marraffini, L. A. CRISPR-Cas immunity in prokaryotes. Nature 526, 55-61 (2015)), which prevent successful delivery of genetic material. Currently, there are no technologies to address this challenge and there is an urgent need for effective methods, especially genetic methods to modify the microbiota. Such methods will be especially useful for personalized microbiome engineering, but may also have industrial uses, such as in agricultural and industrialized livestock settings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Overview of Metagenomic Alteration of Gut microbiome by In situ Conjugation (MAGIC). (a) MAGIC implementation to transfer replicative or integrative pGT vectors from an engineered donor strain into amenable recipients in a complex microbiome. Replicative vectors feature a broad-host range origin of replication (oriR), while integrative vectors contain a transposable Himar cassette and transposase. The donor E. coli strain contains genomically integrated conjugative transfer genes (tra) and a mCherry gene. Transconjugant bacteria are detectable based on expression of an engineered payload that includes GFP and an antibiotic resistance gene (abR).

FIG. 2A through FIG. 2D. Identification and isolation of genetically tractable bacteria from the murine gut using MAGIC. (FIG. 2A) Implementation of MAGIC in a murine model with fecal bacterial analysis by FACS, antibiotic selection, and sequencing. (FIG. 2A) FACS dot plots of fecal bacteria, pre- and post-gavage of EcGT2 donors containing pGT-L3 or pGT-L6 vector libraries. Green boxes define the sorted GFP+/mCherry− transconjugant populations. For each vector library, fecal samples from 3 co-housed mice were independently evaluated by flow cytometry with similar results. (FIG. 2B) Longitudinal analysis of fecal microbiome by flow cytometry for presence of EcGT2 pGT-NT donor cells (red triangles, n=4 mice) and transconjugants of vector libraries pGT-L3 (purple circles, n=3 mice), pGT-L6 (maroon circles, n=3 mice), pGT-NT control (green circles, n=4 mice), or PBS (no donor) control (orange circles, n=2 mice). Donor cells and transconjugants were lost within 48 hours. The dotted line shows the detection limit. (FIG. 2C) 16S taxonomic classification of transconjugants (GFP⁺/mCh⁻) enriched by FACS of pGT-L3 and pGT-L6 recipient groups. Each column represents transconjugants from one mouse. Each OTU's relative abundance in the total bacterial population is shown in the grayscale heat-map, while each OTU's fold enrichment among transconjugants is shown in the orange heat-map. Bracketed values indicate confidence of taxonomic assignment by RDP classifier. Genera with successfully cultivated isolates are denoted by white stars. (FIG. 2D) PCR confirmed the presence of the antibiotic resistance/GFP payload cassette from pGT-L3 and pGT-L6 vectors in diverse isolates that were engineered in the murine gut and isolated by selective plating with carbenicillin or tetracycline. NA indicates 16S sequences that were not available.

FIG. 3A through FIG. 3C. Transconjugant native gut bacteria recolonize the gut and mediate secondary transfer of engineered genetic payloads. (FIG. 3A) Left panel: GFP expression profiles of three isolates (MGB3, MGB4, MGB9; n=5 for each) versus control strain (E. coli MG1655, n=5). MGB isolates were P. mirabilis (orange bar) and E. fergusonii (blue bars) containing either vector pGT-Ah1 (red border) or vector pGT-B1 (purple border). E. fergusonii strains were genetically identical, but received two different vectors. Right panel: efficiency of in vitro conjugation of pGT vectors from MGB strains to E. coli MG1655 recipients. EcGT2 donors were used as positive controls (gray bars). Sample sizes are n=2-4. Bars indicate means; error bars indicate standard deviation. (FIG. 3B) Colonization of MGB strains and EcGT2 lab strain in mice (n=6, n=4 respectively) over time, after initial oral gavage. Cell densities were determined by both plating (light green) and flow cytometry (dark green) of fecal bacteria, and by flow cytometry for E. coli (orange). Error bars indicate standard deviation. (FIG. 3C) FACS enrichment and 16S taxonomic classification of top in vivo transconjugants at 6 hours post-gavage with MGB strains. Fecal samples from 6 mice were combined for analysis. Each OTU's relative abundance in the total bacterial population is shown in the grayscale heat-map, while each OTU's fold enrichment among transconjugants is shown in the orange heat-map. Bracketed values indicate confidence of taxonomic assignment by RDP classifier. Red asterisks denote OTUs that share the same genus as MGB donors.

FIG. 4A through FIG. 4D. Overview of Metagenomic Alteration of Gut microbiome by In situ Conjugation (MAGIC) and plasmid maps of MAGIC vectors. (FIG. 4A) In contrast to traditional approaches to cultivate microbes first and then test for genetic accessibility, MAGIC harnesses horizontal gene transfer in the native environment to genetically modify bacteria in situ. Transconjugant bacteria can be detected by FACS or antibiotic selection and further manipulated. (FIG. 4B) Map of Himar transposon integrative vectors (pGT-Ah and pGT-Kh variants found in libraries L2, L4, L5, L6, L7 and L8). (FIG. 4C) Map of replicative vectors with pBBR1 origin of replication (pGT-B variants found in libraries L1, L4, and L6). (FIG. 4D) Map of replicative vectors with RSF1010 origin of replication (pGT-S variants found in library L3). Although this vector backbone contain genes involved in conjugation (black), these vectors are not self-transmissible^{38, 39}.

FIG. 5A-FIG. 5B. FACS gating methodology for isolation of transconjugant bacteria. (FIG. 5A) Illustration of FACS enrichment method to isolate transconjugant cells from complex recipient populations. GFP and mCherry fluorescence are used to gate cell populations consisting of E. coli donors and diverse recipients. Quadrants Q1 and Q2 correspond to donor cells (mCh⁺), while un-manipulated recipients are in quadrant Q3. Quadrant Q4 contains transconjugants that received the GFP gene cargo and are not naturally mCherry fluorescent (GFP⁺, mCh⁻). Q4 cells are isolated and further analyzed. This gating was used to analyze fecal samples from each individual mouse in each in situ experiment, as well as every in vitro conjugation in this study by flow cytometry. (FIG. 5B) To validate the FACS enrichment method, GFP⁺ E. coli were mixed with a natural murine fecal bacterial community at given levels (1-100% of population) and retrieved by FACS. 16S sequencing of the samples showed that the fluorescent E. coli were efficiently and specifically enriched by FACS. Although the raw Q4 population contained some autofluorescent cells, the only remaining OTU in Q4 after applying an enrichment filter (see Online Methods) was E. coli.

FIG. 6A-FIG. 6B. pGT vectors were transferred from E. coli donors to representative recipient species during in vitro conjugations. (FIG. 6A) In vitro conjugation efficiency of replicative vector pGT-B1 from E. coli donor to various recipients, which are plotted by phylogenetic relationships. (FIG. 6B) In vitro conjugation efficiency of vector pG-Ah1 between E. coli donor and various recipients. This vector is replicative only in Proteobacteria (E. coli, S. enterica, V. cholera, P. aeruginosa) but delivered genetic cargo by transposition into a broader array of bacteria. Asterisks indicate cultures grown in anaerobic conditions, while all other cultures were grown aerobically. Conjugation efficiencies were calculated from 2 independent conjugations.

FIG. 7A through FIG. 7C. pGT vectors were transferred from E. coli donors to murine fecal bacteria during in vitro conjugations. (FIG. 7A) In vitro conjugation of pGT vectors from EcGT2 donor strain into fecal bacteria extracted from murine feces. (FIG. 7B) Aerobic (top) and anaerobic (bottom) conjugations were performed using EcGT2 strains containing no vector (mock conjugation), a nontransferable vector (pGT-NT), pGT-L3, pGT-L7, and pGT-L8. Aerobic conjugations were plated on selective and non-selective media and grown aerobically at 37 C for 24 hours. Anaerobic conjugations were plated on selective and non-selective media, grown anaerobically at 37 C for 48 hours, and exposed to oxygen at room temperature for 48 hours. Red arrows indicate GFP+ CFUs on nonselective plates. (FIG. 7C) Efficiencies of aerobic (top) and anaerobic (bottom) conjugations. Aerobic conjugation efficiencies were calculated from 3 independent conjugations; anaerobic conjugation efficiencies were calculated from 1 conjugation.

FIG. 8A-FIG. 8B. FACS enriches for GFP+, antibiotic-resistant transconjugant gut bacteria arising from in vitro conjugations. (FIG. 8A) Implementation of FACS enrichment of in vitro conjugations. (FIG. 8B) Conjugations between EcGT2 harboring vector libraries pGT-L3, pGT-L7, and pGT-L8 and murine fecal bacteria were performed aerobically overnight. A mock conjugation using EcGT2 with no vector and a negative control conjugation using the pGT-NT non-transferable vector were also performed. 20,000 FACS sorted events from Q3 (mCherry−/GFP−) and Q4 (mCherry−/GFP+) populations were plated on selective and non-selective media and grown aerobically to select for transconjugants. Cultivable aerobic transconjugants of pGT-L3 and pGT-L7 vectors were successfully enriched by FACS, although GFP+ CFUs may appear dim against the autofluorescent media. This experiment was performed independently twice with similar results.

FIG. 9. Fluorescence microscopy of FACS-sorted in vitro conjugations. Overlays of bright-field, GFP, and mCherry channels are shown, alongside GFP and mCherry channels. Q3 populations from unmodified fecal bacteria are negative for both GFP and mCherry, while Q4 populations from aerobic overnight in vitro conjugations of vector libraries pGT-L3 and pGT-L7 show enrichment of GFP-expressing cells as well as some donor cells (mCherry+/GFP−), which were eliminated in downstream sequencing analyses. This experiment was performed independently three times with similar results.

FIG. 10A through FIG. 10C. Identification of FACS-enriched in vitro transconjugants by 16S sequencing. (FIG. 10A) FACS dot plots of in vitro conjugations of murine gut bacteria and EcGT2 donors with vector libraries pGT-L1, L3, and L7. This experiment was performed 3 times with similar results. Green boxes define the sorted GFP⁺/mCherry⁻ transconjugant populations. (FIG. 10B) 16S taxonomic classification of in vitro GFP⁺/mCherry⁻ transconjugants of pGT-L1, L3, and L7 enriched by FACS. Relative abundance of each OTU in the unsorted population is shown in the grayscale heat-map, while fold enrichment for transconjugants of each OTU is shown in the orange heat-map with annotated taxonomic identities. Bracketed values indicate confidence of taxonomic assignment by RDP classifier. Genera with successfully cultivated isolates are denoted by stars. Each column represents FACS-enriched transconjugants from one conjugation. (FIG. 10C) Comparison of OTUs shared between transconjugants arising from each vector library during in vitro conjugations. 18 OTUs were shared between all 3 libraries, with a total of 47 OTUs being shared between at least 2 libraries.

FIG. 11A through FIG. 11D. Identification of FACS-enriched in situ transconjugants by 16S sequencing. (FIG. 11A) Implementation of MAGIC in a murine model with fecal bacterial analysis by FACS, antibiotic selection, and sequencing. (FIG. 11B) FACS dot plots of in situ conjugations using EcGT2 donors with vector libraries pGT-L1, L2, and L3. Green boxes define the sorted GFP⁺/mCherry⁻ transconjugant populations. Each plot shows fluorescence expression of bacteria from the combined fecal samples of 3 co-housed mice. The experiment was run 3 independent times with similar results. (FIG. 11C) 16S taxonomic classification of FACS-enriched transconjugants from in situ mouse experiments using vector libraries pGT-L1, L2, and L3. Relative abundance of each OTU in the unsorted population is shown in the grayscale heat-map, while fold enrichment for transconjugants of each OTU is shown in the orange heat-map with annotated taxonomic identities. Bracketed values indicate confidence of taxonomic assignment by RDP classifier. Each column represents data from a separately housed cohort of 3 mice whose fecal samples were combined for analysis. Genera with successfully cultivated isolates are denoted by stars. (FIG. 11D) The pGT-L3 transconjugant population from (b) was further analyzed by comparing Q4 enriched OTUs against Q3 OTUs, which represent a sample of the GFP-native bacteria population, and by performing enrichment analysis of Q4 samples that were sorted again for Q4. Enriched GFP+ transconjugants were robust whether compared against the total fecal population or against Q3. 7 out of 11 OTUs enriched in Q4 were present in the double-sorted Q4 population, indicating that Q4 sorting is robust. The OTUs lost upon double-sorting were obligate anaerobes and likely sensitive to prolonged aerobic conditions during double-sorting.

FIG. 12A through FIG. 12C. Identification of FACS-enriched in situ transconjugants of multi-vector libraries. (FIG. 12A) Flow cytometric quantification of in situ transconjugants in the total bacterial population, post-gavage of EcGT2 donors containing pGT-L4 (green, n=4 mice) or pGT-L5 (blue, n=4 mice) vector libraries. Control groups gavaged with PBS (black, n=2 mice) or donors containing a non-transferrable pGT-NT vector (red, n=2 mice) produced no detectable transconjugants. Black bars indicate means. (FIG. 12B) Longitudinal analysis of murine fecal microbiome by flow cytometry for presence of transconjugants post-gavage of EcGT2 donors containing pGT-L4 (green, n=6 mice), or pGT-L5 (blue, n=6 mice). Donor cells of these libraries (orange, n=12 mice) were lost within 48 hours, while transconjugants were observed up to 72 hours post-gavage. The dotted line indicates the detection limit of flow cytometry. Error bars indicate standard deviation. (FIG. 12C) 16S taxonomic classification of transconjugants (GFP⁺/mCh⁻) enriched by FACS of pGT-L4 and pGT-L5 recipient groups. Relative abundance of each OTU in the unsorted population is shown in the grayscale heat-map on the left, while fold enrichment for transconjugants of each OTU is shown in the orange heat-map on the right with annotated taxonomic identities. Bracketed values indicate confidence of taxonomic assignment by RDP classifier. Each column represents data from 6 mice from 2 independent cohorts whose fecal samples were combined for analysis. Genera with successfully cultivated isolates are denoted with stars.

FIG. 13A through FIG. 13D. Identification of FACS-enriched in situ transconjugants in mice from a different commercial vendor. (FIG. 13A) FACS dot plots of in situ conjugations using EcGT2 pGT-L3 donors in a cohort of mice from a different vendor (Charles River Laboratories). Green boxes define the sorted GFP+/mCherry− transconjugant populations. Flow cytometry was performed 3 times, on fecal samples from individual co-housed mice, with similar results. (FIG. 13B) 16S taxonomic classification of FACS-enriched GFP+/mCherry− transconjugants of pGT-L3. Relative abundance of each OTU in the unsorted population is shown in the grayscale heat-map, while fold enrichment for transconjugants of each OTU is shown in the orange heat-map with annotated taxonomic identities. Bracketed values indicate confidence of taxonomic assignment by RDP classifier. Each column represents bacteria from one mouse. Genera with successfully cultivated isolates are denoted by stars. (FIG. 13C) Metagenomic 16S rRNA sequencing of mouse fecal samples shows that mice from different vendors have divergent gut microbiomes, with some shared OTUs. (FIG. 13D) In in situ experiments using the same vector library (pGT-L6) in cohorts of 3 mice each from different vendors, 10 transconjugant OTUs were shared between cohorts.

FIG. 14. PCR-validated transconjugant isolates from in situ mouse experiments. 297 PCR-validated isolates from in situ experiments using vector libraries pGT-L3 and pGT-L6 were identified by 16S Sanger sequencing and assigned to a genus using RDP classifier with assignment confidence >0.89.

FIG. 15A through FIG. 15G. Comparison of vector and payload stability in two transconjugant isolates. (FIG. 15A) Vector map of pGT-B1. GFP and beta-lactamase genes are expressed from separate promoters on a replicative pBBR1 origin plasmid. (FIG. 15B) MGB4, an Escherichia fergusonii isolate containing pGT-B1, lost GFP expression over time when serially passaged without selection for 15 days. Plating was performed for 3 independent serial passages. (FIG. 15C) Quantification of carb-resistant and GFP+ CFUs of MGB4 over time; all CFUs remained carb-resistant as the population lost GFP expression. Center values are the means of 3 serial passages; error bars represent standard deviation. (FIG. 15D) Colony PCR for the pGT-B1 backbone showed that the plasmid was absent in GFP− CFUs at all time points surveyed. Each lane shows the PCR product for one colony. This PCR was performed once. (FIG. 15E) Vector map of pGT-Ah1, which contains GFP and beta-lactamase genes on a transposable cassette. The plasmid backbone contains a chloramphenicol resistance gene for selection. (FIG. 15F) MGB9, an Escherichia fergusonii isolate containing pGT-Ah1, remained 100% GFP+ during serial passaging without selection over 11 days. Plating was performed for 3 independent serial passages. (FIG. 15G) Over time the proportion of MGB9 CFUs expressing the genes on the transposable cassette (GFP+and carb-resistant) remained at 100%, while the chloramphenicol resistance conferred by the pGT-Ah1 backbone was lost in some of the population. Center values are the means of 3 serial passages; error bars represent standard deviation.

FIG. 16A through FIG. 16D. Characterization of 3 Modifiable Gut Bacteria (MGB) strains by whole-genome sequencing and in vitro conjugation. (FIG. 16A) Three distinct MGB strains, isolated from in vitro conjugations between E. coli pGT donors and murine fecal bacteria, were analyzed by whole-genome sequencing. MGB4 and MGB9 appear to be the same strain isolated from separate experiments with different pGT vectors transferred. Sequencing of (FIG. 16B) MGB4/9 and (FIG. 16C) MGB3 revealed the presence of genes involved in conjugation and genetic transfer. However, only MGB4/9 strains that shared homology with the pECO-fce plasmid were observed to transfer their pGT vectors to E. coli during in vitro conjugations. (FIG. 16D) PCR confirmation of pGT vector transfer from MGB4 to an E. coli recipient following in vitro conjugation. The conjugation was performed 3 times with similar results; 5 individual transconjugants were assessed by colony PCR.

FIG. 17A- FIG. 17B. Longevity of donor E. coli strains in the murine gut following oral gavage. (FIG. 17A) In vivo gut colonization profiles of MAGIC donors EcGT1 (S17, galK::mCherry), EcGT2 (S17, asd::mCherry), and control E. coli MG1655 in C57BL/6 mice measured by flow cytometry of fecal bacteria after a single gavage of 10⁹ cells. Mean values were calculated using feces from 2 gavaged mice; error bars indicate standard deviation. (FIG. 17B) Two orally gavaged doses of 10⁹ EcGT1 cells resulted in a longer persistence of this donor in the gut. Mean values were calculated using feces from 2 gavaged mice; error bars indicate SEM.

FIG. 18A through FIG. 18D. Characterization of MGB recolonization of the murine gut. (FIG. 18A) Schematic diagram of experiment: genetically tractable gut microbiota were isolated from the murine microbiome in vitro and then orally gavaged to recolonize the gut. (FIG. 18B) MGB3, MGB4, and MGB9 strains orally gavaged into mice (n=4) as a mixture recolonized the GI tract without any antibiotic treatment. MGBs were detectable in fecal samples for at least 15 days post-gavage. (FIG. 18C) MGB strains (namely MGB4) were present in all sampled locations along the GI tract when the mice (n=4) were euthanized 15 days post-gavage. Error bars represent standard deviation. (FIG. 18D) Phylogenetic tree of FACS-sorted GFP⁺/mCherry⁻.transconjugants in fecal samples from mice after 11 days post-gavage of MGB strains. Fecal samples from 4 mice were combined for analysis. Relative abundance of each OTU in the unsorted population is shown in the grayscale heat-map, while fold enrichment for transconjugants of each OTU is shown in the orange heat-map. Bracketed values indicate confidence of taxonomic assignment by RDP classifier. The red asterisk denotes the Escherichia/Shigella OTU that shares a genus with the MGB4/9 donors.

### DETAILED DESCRIPTION

Engineering microbial populations in open environments is an outstanding challenge. Described herein are methods and materials to modify genetically tractable yet undomesticated microbes from complex microbial communities. Certain aspects relate to utilizing engineered horizontal gene transfer elements to genetically alter cells of microbiome and/or involve high-throughput selection strategies to tag and retrieve genetically modified native commensal strains from the mammalian gut. Further, methods are described wherein isolated bacteria from the mammalian gut microbiome that were amenable to genetic manipulation are redeployed back into the mammalian subject as host-optimized engineerable probiotics.

Other embodiments relate to a modular mobile plasmid vector that is assembled with multiple components to inure the plasmid with the ability to transfer a payload of interest to cells of a microbiome. Components of a replicative plasmid vector embodiment may include at least one origin of replication (oriR) sequence; an origin of transfer sequence; a payload sequence of interest; and a regulatory sequence linked with the payload sequence so as to control expression of the payload sequence of interest. An integrative plasmid vector embodiment may include at least one origin of replication (oriR) sequence; an origin of transfer sequence; a transposase sequence; a payload sequence of interest; a regulatory sequence linked with the payload sequence so as to control expression of the payload sequence; and a first and second transposon-associated recognition sequence (e.g. inverted repeat (IR) sequence) positioned upstream and downstream of the payload sequence, respectively.

The plasmid vectors may include further optional components depending on the intended use/objectives, such as selection markers (e.g. antibiotic selection genes, auxotrophic markers, etc.) helpful for selection of donor cells, as well as markers designed for selection of recipient cells (e.g. fluorescent markers). In a specific example, the plasmid vector may be based on a pGT backbone, possessing a number of modular components as set forth in the diagram below:

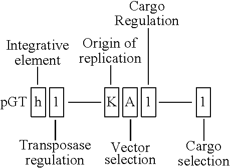

As will be further explained herein, the modular plasmid vector may be designed for replication within the recipient cell or designed to be integrative, i.e., the payload of interest is chromosomally integrated in the recipient cell. For replicative plasmid vectors, these do not need the integrative element and transposase regulation components. Further, in certain embodiments, the plasmid vector need not include certain section marker sequences. The selection markers are helpful for methods that involve identifying and isolating tractable strains from the microbiome environment, as will be discussed further below. Yet in further embodiments, disclosed are microbial shuttles that are engineered to carry and deliver a payload of interest to tractable cells of a microbiome. Examples for each of the components and the codes used in the plasmid nomenclature are provided in Table 1, and sequences are provided in Table 3. In addition to the oriR examples provided in Tables 1 and 3, the oriR may also be pBBR1, OriV, R6K, p15A, pBI143, Inc (IncP, IncX, IncF, Inc), Col and RS1010. Sequences and other background information for these alternative oriR components are known, see Microbiol Mol Biol Rev 1998 52:434-464 and Jain, FEMS Microbiology Letters, 2013 348:87-96, incorporated by reference. Examples of oriT examples include but are not limited to RK2 and F. Other oriT examples could be used as are taught in Li et al., Nucleic Acids Research, 2018, 46:W229-W234 and parts.igem.org/conjugation. Regulatory sequences (e.g. promoters and enhancers) can be any suitable known promoter. Common bacterial promoters useful in plasmids include T7, T7lac, Sp6, araBAD, trp, lac, Ptac, pL, and T3. For further background, see Chen et al., Nat Commun 9, 64 (2018) and Kent R, Dixon N., “Contemporary Tools for Regulating Gene Expression in Bacteria”, Trends Biotechnol, 2019, doi: 10.1016/j.tibtech.2019.09.007.

According to other embodiments, disclosed is a method for altering a microbiome of a subject (e.g. human or non-human animal). In a specific example, the method allows for identifying recipient strains that received a plasmid vector as described herein. In an even more specific example, the method involves the steps of: (a) providing a donor bacterial strain, wherein the donor bacterial strain comprises a genomically integrated conjugation system or an episomal system, a fluorophore gene and is optionally auxotropic for at least one compound; (b) introducing a plasmid vector to the donor bacterial strain, wherein the plasmid vector comprises an origin of replication (oriR), an origin of transfer (oriT), an antibiotic selection gene, and sfGFP gene; or the plasmid vector comprises an OriR, and OriT, a Himar transposon comprising an antibiotic selection gene, sfGFP gene and a Himar transposase gene; (c) selecting recipient bacterial strains that had incorporated the plasmid vector by antibiotic selection or Fluorescence Activated Cell Sorting (FACS); and, (d) optionally recolonizing the subject with recipient bacteria from step (c).

The method may further involve the steps of: (i) isolating gut bacteria from the subject to provide a recipient bacterial strain after step (b); and, (ii) mixing the donor and recipient bacterial strains. As used herein, the term “donor bacterial strain”, “microbial shuttle”, “shuttle vector” and “shuttle” are used interchangeably.

In one embodiment, the fluorophore gene may be mCherry, mTurquoise2, GFP, mTagBFP2, mCerulean3, EGFP, mWasabi, mNeonGreen, mClover3, Venus, Citrine, mKOk, tdTomato, TagRFP-T, mRuby3, mScarlet, FusionRed, mStable, mKate2, mMaroon1, mCardinal, T-Sapphire, mCyRFP1, and/or LSSSmOrange, and the donor bacterial strain is auxotrophic for a metabolite (e.g. diaminopimelic acid), the conjugation system is RP4, R1, F conjugation or, or pKM101 and the origin of replication (oriR) is selected from the group consisting of pBR1, OriV, R6K, p15A, pBI143 and RS1010.

In another embodiment, the origin of transfer (oriT) is Rk2.

The antibiotic selection gene may encode resistance to one of the following antibiotic selection agents: carbenicillen, beta-lactamase, chloramphenicol, tetracycline, spectinomycin, kanamycin, or gentamycin. The antibiotic selection of step (e) may comprise selection with carbenicillen, beta-lactamase, chloramphenicol, tetracycline, spectinomycin, kanamycin, or gentamycin.

In one embodiment, the donor bacterial strain is a gram negative or gram positive bacterial strain. For example, the donor bacterial strain may be a strain of Escherichia coli, or a strain of Shigella.

The subject may be a human or non-human animal from any environment including aquatic and terrestrial environments.

The recipient bacterial strain may be one or more phyla selected from the group of phyla consisting of Bacteriodetes, Proteobacteria, Fusobacteria, Actinobacteria, Deferribacteres, Tenericutes, Planctomycetes, and Firmicutes. In another embodiment, the recipient bacterial strain is of an order selected from the group consisting of Bacteroidales, Cytophagales, Flavobacteriales, Fusobacteriales, Verrucomicrrobia, Xanthomonadales, Neisseriales, Burkholderiales, Psudomonadales, Pasteurelales, Enterobacteriales, Deinococcales, Conobacteriales, Solirubrobacterales, Actinomycetales, Sphingomonadales, Rhizobiales, Selenomonadales, Lactobcillales, Erysipelotrichales, Bacillales, and Clostridiales and mixtures thereof.

Data is presented herein demonstrating in situ transfer of synthetic gene circuits directly into the native microbiome in a live animal to actuate new functions. Moreover, host-adapted probiotics are disclosed that, in contrast to the traditional non-gut-adapted ones, can be readily and stably reestablished back into their native gut microbiome, thus providing a new tool for specific and long-term studies with a more personalized medicine approach.

Using engineered horizontal gene transfer and transposon systems, bacteria were successfully identified and isolated from the mammalian gut microbiome that were amenable to genetic manipulation and then redeployed them back into the mammalian subject as host-optimized engineerable probiotics. Data is provided demonstrating in situ gene transfer in a live animal to deliver new traits into its established gut microbiota. This in situ genome engineering approach enables the accelerated development of new microbial chasses for synthetic biology and the introduction of novel capabilities into established microbial communities with minimal disruption to their native milieu in order to explore community dynamics and host interactions.

Further embodiments pertain to cells into which plasmid vectors described herein have been introduced. The cell may be capable of colonizing within the subject and generating their payload sequence of interest, or deliver their payload of interest to commensal bacteria within a microbiome of the subject. Examples of payload sequences of interest include those that encode any beneficial or therapeutic polypeptides. Payload sequences of interest may also include sequences that encode antisense, siRNA, shRNA, or other RNA interfering molecules that are delivered to a cell in the microbiome or in in the natural environment for silencing expression of a targeted gene. Payload sequences may also include those that encodes various components of CRISPR/Cas machinery, restriction endonucleases and the like to make further genetic modifications in a recipient cell.

Other embodiments pertain to compositions that include one or more donor bacterial cells that include a plasmid vector as described herein. Such compositions may be formulated for administration to a subject, including, but not limited to, topical, oral or inhalatory routes of administration. The donor bacterial cells of the compositions can include plasmid vectors that comprise a payload sequence of interest that serves a beneficial and/or therapeutic purpose.

### Definitions

In accordance with the present invention, there may be numerous tools and techniques within the skill of the art, such as those commonly used in molecular immunology, cellular immunology, pharmacology, and microbiology. See, e.g., Sambrook et al. (2001) Molecular Cloning: A Laboratory Manual. 3rd ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

“Treating” or “treatment” of a state, disorder or condition includes:

(1) preventing or delaying the appearance of clinical symptoms of the state, disorder, or condition developing in a subject who may be afflicted with or predisposed to the state, disorder or condition but does not yet experience or display clinical symptoms of the state, disorder or condition; or

(2) inhibiting the state, disorder or condition, i.e., arresting, reducing or delaying the development of the disease or a relapse thereof (in case of maintenance treatment) or at least one clinical symptom, sign, or test, thereof; or

(3) relieving the disease, i.e., causing regression of the state, disorder or condition or at least one of its clinical or sub-clinical symptoms or signs.

The benefit to a subject to be treated is either statistically significant or at least perceptible to the patient or to the physician.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

An “immune response” refers to the development in the host of a cellular and/or antibody-mediated immune response to a composition or vaccine of interest. Such a response usually consists of the subject producing antibodies, B cells, helper T cells, suppressor T cells, regulatory T cells, and/or cytotoxic T cells directed specifically to an antigen or antigens included in the composition or vaccine of interest.

A “therapeutically effective amount” means the amount of a compound, cells, or compositions containing cells that, when administered to a subject are capable genetically altering cells of a microbiome in the subject. Further, a therapeutically effective amount may be an amount of a compound, cells, or cell-containing composition, for treating a state, disorder or condition, is sufficient to effect such treatment.

As used herein, “payload sequence of interest” relates to any sequence encoding a payload. A payload sequence of interest are typically, but not necessarily, heterologous to the cell into which they are introduced.

As used herein, the term “payload” refers to a peptide, polypeptide, protein, DNA and/or RNA sequence. Examples of payloads include, but are not limited to, therapeutic proteins, RNA interfering molecules, selectable markers (positive or negative e.g. auxotrophy, prototrophy or antibiotic resistance), reporter (e.g. fluorophore), and/or or nucleic acid sequences involved in genetic manipulation such as guide RNA sequences. Examples of reporter genes is found in Thorn, Mol Biol Cell, 2017, 28:848-857 incorporated herein. Examples antibiotic resistance markers include, but are not limited to, genes that confer resistance to Ampicillin, Carbenicillin, Chloramphenicol, hygromycin B, Kanamycin, Spectinomycin, or Tetracyline. The term “payload of interest” refers to the payload encoded by the payload sequence of interest. At certain locations herein, the terms “payload” and “cargo” are used interchangeably. Examples of auxotrophic and prototrophic markers are described in U.S. Pat. No. 9,243,253, incorporated herein.

The term “therapeutic protein” refers to a polypeptide that affects or effects a cellular or molecular function within the cell in which they are expressed, or are released by the cell in which they are expressed to effect a function in a host or environment. Examples of therapeutic proteins include but are not limited to antibodies, cytokines, growth factors, transcription factors, enzymes, components of genetic manipulation machinery such as RNA-programmable nucleases (e.g. CAS proteins). The term “enzymes” can include a broad range of beneficial enzymes including, but not limited to, enzymes intended to treat a disease, deficiency or disorder in a subject, an enzyme related to cellular process of cells in the microbiome such as enzymes related to synthesis of a metabolite (nucleic acids, fatty acids, amino acids, vitamins, autoinducers, etc.) or digestion of metabolites.

The term “percent identity” means the percentage determined by the direct comparison of two sequences (nucleic or protein) by determining the number of nucleic acids or amino acid residues common to both sequences, then dividing this by the number of nucleic acids or amino acid residues in the longer of the two sequences and multiplying the result by 100. Alignment, for purposes of determining percent identity, can be achieved in various ways that are within the skill in the art, for instance, using publicly available computer software such as BLAST, ALIGN, or Megalign (DNASTAR) software. Percent identity of two sequences can be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

While it is possible to use a composition provided by the present invention for therapy as is, it may be preferable to administer it in a pharmaceutical formulation, e.g., in admixture with a suitable pharmaceutical excipient, diluent or carrier selected with regard to the intended route of administration and standard pharmaceutical practice. Accordingly, in one aspect, the present invention provides a pharmaceutical composition or formulation comprising at least one active composition, or a pharmaceutically acceptable derivative thereof, in association with a pharmaceutically acceptable excipient, diluent and/or carrier. The excipient, diluent and/or carrier must be “acceptable” in the sense of being compatible with the other ingredients of the formulation and not deleterious to the recipient thereof.

The compositions of the invention can be formulated for administration in any convenient way for use in human or veterinary medicine. The invention therefore includes within its scope pharmaceutical compositions comprising a product of the present invention that is adapted for use in human or veterinary medicine.

In a preferred embodiment, the pharmaceutical composition is conveniently administered as an oral formulation. Oral dosage forms are well known in the art and include tablets, caplets, gelcaps, capsules, and medical foods. Tablets, for example, can be made by well-known compression techniques using wet, dry, or fluidized bed granulation methods.

Such oral formulations may be presented for use in a conventional manner with the aid of one or more suitable excipients, diluents, and carriers. Pharmaceutically acceptable excipients assist or make possible the formation of a dosage form for a bioactive material and include diluents, binding agents, lubricants, glidants, disintegrants, coloring agents, and other ingredients. Preservatives, stabilizers, dyes and even flavoring agents may be provided in the pharmaceutical composition. Examples of preservatives include sodium benzoate, ascorbic acid and esters of p-hydroxybenzoic acid. Antioxidants and suspending agents may be also used. An excipient is pharmaceutically acceptable if, in addition to performing its desired function, it is non-toxic, well tolerated upon ingestion, and does not interfere with absorption of bioactive materials.

Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

As used herein, the phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopoeia or other generally recognized pharmacopeias for use in animals, and more particularly in humans.

“Patient” refers to mammals and includes human and veterinary subjects.

The dosage of the therapeutic formulation will vary widely, depending upon the nature of the disease, the patient's medical history, the frequency of administration, the manner of administration, the clearance of the agent from the subject, and the like. The initial dose may be larger, followed by smaller maintenance doses. The dose may be administered as infrequently as weekly or biweekly, or fractionated into smaller doses and administered daily, semi-weekly, etc., to maintain an effective dosage level. In some cases, oral administration will require a higher dose than if administered intravenously. In some cases, topical administration will include application several times a day, as needed, for a number of days or weeks in order to provide an effective topical dose.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, olive oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

The terms “treat”, “treatment”, and the like refer to a means to slow down, relieve, ameliorate or alleviate at least one of the symptoms of the disease, or reverse the disease after its onset.

The terms “prevent”, “prevention”, and the like refer to acting prior to overt disease onset, to prevent the disease from developing or minimize the extent of the disease or slow its course of development.

The term “agent” as used herein means a substance that produces or is capable of producing an effect and would include, but is not limited to, chemicals, pharmaceuticals, biologics, small organic molecules, antibodies, nucleic acids, peptides, and proteins.

As used herein, the term “isolated” and the like means that the referenced material is free of components found in the natural environment in which the material is normally found. In particular, isolated biological material is free of cellular components. In the case of nucleic acid molecules, an isolated nucleic acid includes a PCR product, an isolated mRNA, a cDNA, an isolated genomic DNA, or a restriction fragment. In another embodiment, an isolated nucleic acid is preferably excised from the chromosome in which it may be found. Isolated nucleic acid molecules can be inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated material may be, but need not be, purified.

The term “purified” and the like as used herein refers to material that has been isolated under conditions that reduce or eliminate unrelated materials, i.e., contaminants. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

The terms “expression profile” or “gene expression profile” refers to any description or measurement of one or more of the genes that are expressed by a cell, tissue, or organism under or in response to a particular condition. Expression profiles can identify genes that are up-regulated, down-regulated, or unaffected under particular conditions. Gene expression can be detected at the nucleic acid level or at the protein level. The expression profiling at the nucleic acid level can be accomplished using any available technology to measure gene transcript levels. For example, the method could employ in situ hybridization, Northern hybridization or hybridization to a nucleic acid microarray, such as an oligonucleotide microarray, or a cDNA microarray. Alternatively, the method could employ reverse transcriptase-polymerase chain reaction (RT-PCR) such as fluorescent dye-based quantitative real time PCR (TaqMan® PCR). In the Examples section provided below, nucleic acid expression profiles were obtained using Affymetrix GeneChip® oligonucleotide microarrays. The expression profiling at the protein level can be accomplished using any available technology to measure protein levels, e.g., using peptide-specific capture agent arrays.

The terms “gene”, “gene transcript”, and “transcript” are used somewhat interchangeable in the application. The term “gene”, also called a “structural gene” means a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription. “Transcript” or “gene transcript” is a sequence of RNA produced by transcription of a particular gene. Thus, the expression of the gene can be measured via the transcript.

The term “antisense DNA” is the non-coding strand complementary to the coding strand in double-stranded DNA.

The term “genomic DNA” as used herein means all DNA from a subject including coding and non-coding DNA, and DNA contained in introns and exons.

The term “nucleic acid hybridization” refers to anti-parallel hydrogen bonding between two single-stranded nucleic acids, in which A pairs with T (or U if an RNA nucleic acid) and C pairs with G. Nucleic acid molecules are “hybridizable” to each other when at least one strand of one nucleic acid molecule can form hydrogen bonds with the complementary bases of another nucleic acid molecule under defined stringency conditions. Stringency of hybridization is determined, e.g., by (i) the temperature at which hybridization and/or washing is performed, and (ii) the ionic strength and (iii) concentration of denaturants such as formamide of the hybridization and washing solutions, as well as other parameters. Hybridization requires that the two strands contain substantially complementary sequences. Depending on the stringency of hybridization, however, some degree of mismatches may be tolerated. Under “low stringency” conditions, a greater percentage of mismatches are tolerable (i.e., will not prevent formation of an anti-parallel hybrid).

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Vectors include, but are not limited to, plasmids, phages, and viruses.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. A “cassette” refers to a DNA coding sequence or segment of DNA which codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct” or “gene construct.” A common type of vector is a “plasmid”, which generally is a self-contained molecule of double-stranded DNA, usually of bacterial origin, that can readily accept additional (foreign) DNA and which can readily introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes, and may be from the same or different organisms. A large number of vectors, including plasmid and fungal vectors which replicate or exist episomally, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g. antibiotic resistance, and one or more expression cassettes.

The term “host cell” means any cell of any organism that is selected, modified, transformed, grown, used or manipulated in any way, for the production of a substance by the cell, for example, the expression by the cell of a gene, a DNA or RNA sequence, a protein or an enzyme. Host cells can further be used for screening or other assays, as described herein.

A “polynucleotide” or “nucleotide sequence” is a series of nucleotide bases (also called “nucleotides”) in a nucleic acid, such as DNA and RNA, and means any chain of two or more nucleotides. A nucleotide sequence typically carries genetic information, including the information used by cellular machinery to make proteins and enzymes. These terms include double or single stranded genomic and cDNA, RNA, any synthetic and genetically manipulated polynucleotide, and both sense and anti-sense polynucleotide. This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases, for example thio-uracil, thio-guanine and fluoro-uracil.

“Nucleic acid” refers to deoxyribonucleotides or ribonucleotides and polymers thereof in either single- or double-stranded form. The nucleic acids herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, and carbamates) and with charged linkages (e.g., phosphorothioates, and phosphorodithioates). Polynucleotides may contain one or more additional covalently linked moieties, such as, for example, proteins (e.g., nucleases, toxins, antibodies, signal peptides, and poly-L-lysine), intercalators (e.g., acridine, and psoralen), chelators (e.g., metals, radioactive metals, iron, and oxidative metals), and alkylators. The polynucleotides may be derivatized by formation of a methyl or ethyl phosphotriester or an alkyl phosphoramidate linkage. Modifications of the ribose-phosphate backbone may be done to facilitate the addition of labels, or to increase the stability and half-life of such molecules in physiological environments. Nucleic acid analogs can find use in the methods of the invention as well as mixtures of naturally occurring nucleic acids and analogs. Furthermore, the polynucleotides herein may also be modified with a label capable of providing a detectable signal, either directly or indirectly. Exemplary labels include radioisotopes, fluorescent molecules, and biotin.

The term “polypeptide” as used herein means a compound of two or more amino acids linked by a peptide bond. “Polypeptide” is used herein interchangeably with the term “protein.”

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

In the context of genetic transfer between two strains or species of bacteria, the terms “conjugation,” “conjugated,” and “conjugation” refer to the horizontal transfer of genetic material between two cells by direct contact via a pilus. Conjugation, along with viral transduction and transduction, is one of three modes by which DNA can move horizontally between members of a microbial community.

The term “consensus sequence,” as used herein in the context of nucleic acid sequences, refers to a calculated sequence representing the most frequent nucleotide residues found at each position in a plurality of similar sequences. Typically, a consensus sequence is determined by sequence alignment in which similar sequences are compared to each other and similar sequence motifs are calculated. In the context of transposase target site sequences, a consensus sequence of a transposase target site may, in some embodiments, be the sequence most frequently bound, or bound with the highest affinity, by a given transposase.

The term “engineered,” as used herein refers to a protein molecule, a nucleic acid, complex, substance, cell or entity that has been designed, produced, prepared, synthesized, and/or manufactured by a human. Accordingly, an engineered product is a product that does not occur in nature.

The term “effective amount,” as used herein, refers to an amount of a biologically active agent that is sufficient to elicit a desired biological response. For example, in some embodiments, an effective amount of a nuclease may refer to the amount of the nuclease that is sufficient to induce cleavage of a target site specifically bound and cleaved by the nuclease. In some embodiments, an effective amount of a transposase may refer to the amount of the transposase that is sufficient to induce transposition at a target site specifically bound and recombined by the transposase. As will be appreciated by the skilled artisan, the effective amount of an agent, e.g., a nuclease, a transposase, a hybrid protein, a fusion protein, a protein dimer, a complex of a protein (or protein dimer) and a polynucleotide, or a polynucleotide, may vary depending on various factors as, for example, on the desired biological response, the specific allele, genome, target site, cell, or tissue being targeted, and the agent being used.

The term “homologous,” as used herein is an art-understood term that refers to nucleic acids or polypeptides that are highly related at the level of nucleotide and/or amino acid sequence. Nucleic acids or polypeptides that are homologous to each other are termed “homologues.” Homology between two sequences can be determined by sequence alignment methods known to those of skill in the art. In accordance with the invention, two sequences are considered to be homologous if they are at least about 50-60% identical, e.g., share identical residues (e.g., amino acid residues) in at least about 50-60% of all residues comprised in one or the other sequence, at least about 70% identical, at least about 80% identical, at least about 90% identical, at least about 95% identical, at least about 98% identical, at least about 99% identical, at least about 99.5% identical, or at least about 99.9% identical, for at least one stretch of at least 20, at least 30, at least 40, at least 50, at least 60, at least 70, at least 80, at least 90, at least 100, at least 120, at least 150, or at least 200 amino acids.

The term “linker,” as used herein, refers to a chemical group or a molecule linking two adjacent molecules or moieties, e.g., a binding domain (e.g., dCas9) and a transposase domain (e.g., Himar). In some embodiments, a linker joins a nuclear localization signal (NLS) domain to another protein (e.g., a Cas9 protein or a transposase or a fusion thereof). In some embodiments, a linker joins a gRNA binding domain of an RNA-programmable nuclease and the catalytic domain of a transposase. In some embodiments, a linker joins a dCas9 and a transposase. Typically, the linker is positioned between, or flanked by, two groups, molecules, or other moieties and connected to each one via a covalent bond, thus connecting the two. In some embodiments, the linker is an amino acid or a plurality of amino acids (peptide linker). In some embodiments, the linker is an organic molecule, group, polymer, or chemical moiety. In some embodiments, the peptide linker is any stretch of amino acids having at least 1, at least 2, at least 3, at least 4, at least 5, at least 6, at least 7, at least 8, at least 9, at least 10, at least 15, at least 20, at least 25, at least 30, at least 40, at least 50, or more amino acids. In some embodiments, the peptide linker comprises repeats of the tri-peptide Gly-Gly-Ser, e.g., comprising the sequence (GGS)ₙ, wherein n represents at least 1, 2, 3, 4, 5, 6, 7, 8, 9, 10, or more repeats. In some embodiments, the linker comprises the sequence (GGS)₆ (SEQ ID NO: 11). In some embodiments, the peptide linker is the 16 residue “XTEN” linker, or a variant thereof (See, e.g., the Examples; and Schellenberger et al. A recombinant polypeptide extends the in vivo half-life of peptides and proteins in a tunable manner. Nat. Biotechnol. 27, 1186-1190 (2009)).

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The term “nuclease,” as used herein, refers to an agent, for example, a protein, capable of cleaving a phosphodiester bond connecting two nucleotide residues in a nucleic acid molecule. In some embodiments, “nuclease” refers to a protein having an inactive DNA cleavage domain, such that the nuclease is incapable of cleaving a phosphodiester bond. In some embodiments, a nuclease is a protein, e.g., an enzyme that can bind a nucleic acid molecule and cleave a phosphodiester bond connecting nucleotide residues within the nucleic acid molecule. A nuclease may be an endonuclease, cleaving a phosphodiester bonds within a polynucleotide chain, or an exonuclease, cleaving a phosphodiester bond at the end of the polynucleotide chain. In some embodiments, a nuclease is a site-specific nuclease, binding and/or cleaving a specific phosphodiester bond within a specific nucleotide sequence, which is also referred to herein as the “recognition sequence,” the “nuclease target site,” or the “target site.” In some embodiments, a nuclease is a RNA-guided (i.e., RNA-programmable) nuclease, which is associated with (e.g., binds to) an RNA (e.g., a guide RNA, “gRNA”) having a sequence that complements a target site, thereby providing the sequence specificity of the nuclease. In some embodiments, a nuclease recognizes a single stranded target site, while in other embodiments, a nuclease recognizes a double-stranded target site, for example, a double-stranded DNA target site. The target sites of many naturally occurring nucleases, for example, many naturally occurring DNA restriction nucleases, are well known to those of skill in the art. In many cases, a DNA nuclease, such as EcoRI, HindIII, or BamHI, recognize a palindromic, double-stranded DNA target site of 4 to 10 base pairs in length, and cut each of the two DNA strands at a specific position within the target site. Some endonucleases cut a double-stranded nucleic acid target site symmetrically, i.e., cutting both strands at the same position so that the ends comprise base-paired nucleotides, also referred to herein as blunt ends. Other endonucleases cut a double-stranded nucleic acid target sites asymmetrically, i.e., cutting each strand at a different position so that the ends comprise unpaired nucleotides. Unpaired nucleotides at the end of a double-stranded DNA molecule are also referred to as “overhangs,” e.g., as “5′-overhang” or as “3′-overhang,” depending on whether the unpaired nucleotide(s) form(s) the 5′ or the 5′ end of the respective DNA strand. Double-stranded DNA molecule ends ending with unpaired nucleotide(s) are also referred to as sticky ends, as they can “stick to” other double-stranded DNA molecule ends comprising complementary unpaired nucleotide(s). A nuclease protein typically comprises a “binding domain” that mediates the interaction of the protein with the nucleic acid substrate, and also, in some cases, specifically binds to a target site, and a “cleavage domain” that catalyzes the cleavage of the phosphodiester bond within the nucleic acid backbone. In some embodiments a nuclease protein can bind and cleave a nucleic acid molecule in a monomeric form, while, in other embodiments, a nuclease protein has to dimerize or multimerize in order to cleave a target nucleic acid molecule. Binding domains and cleavage domains of naturally occurring nucleases, as well as modular binding domains and cleavage domains that can be fused to create nucleases binding specific target sites, are well known to those of skill in the art.

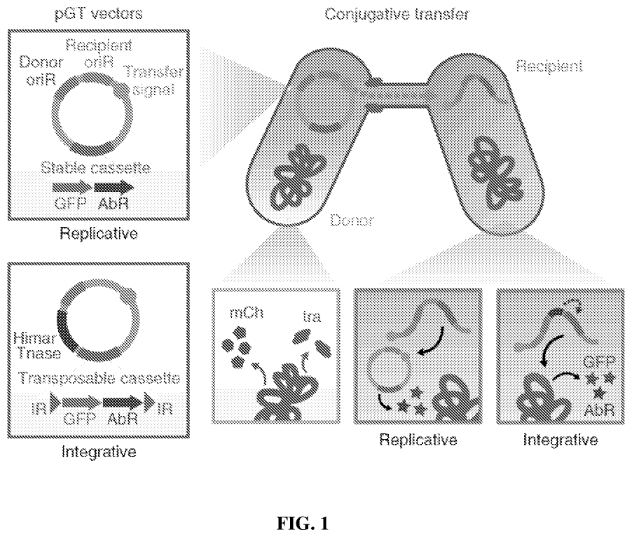

The term “RNA-programmable nuclease,” and “RNA-guided nuclease” are used interchangeably herein and refer to a nuclease that forms a complex with (e.g., binds or associates with) one or more RNA that is not a target for cleavage. In some embodiments, an RNA-programmable nuclease, when in a complex with an RNA, may be referred to as a nuclease: RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 complex to the target); and (2) a domain that binds a Cas9 protein. In some embodiments, domain (2) corresponds to a sequence known as a tracrRNA, and comprises a stem-loop structure. For example, in some embodiments, domain (2) is homologous to a tracrRNA as depicted in FIG. 1E of Jinek et al., Science 337:816-821(2012), the entire contents of which is incorporated herein by reference. Other examples of gRNAs (e.g., those including domain 2) can be found in U.S. Provisional Patent Application, U.S. Ser. No. 61/874,682, filed Sep. 6, 2013, entitled “Switchable Cas9 Nucleases And Uses Thereof,” U.S. Provisional Patent Application, U.S. Ser. No. 61/874,746, filed Sep. 6, 2013, entitled “Delivery System For Functional Nucleases;” PCT Application WO 2013/176722, filed Mar. 15, 2013, entitled “Methods and Compositions for RNA-Directed Target DNA Modification and for RNA-Directed Modulation of Transcription;” and PCT Application WO 2013/142578, filed Mar. 20, 2013, entitled “RNA-Directed DNA Cleavage by the Cas9-crRNA Complex;” the entire contents of each are hereby incorporated by reference in their entirety. Still other examples of gRNAs and gRNA structure are provided herein. See e.g., the Examples. In some embodiments, a gRNA comprises two or more of domains (1) and (2), and may be referred to as an “extended gRNA.” For example, an extended gRNA will e.g., bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease: RNA complex. In some embodiments, the RNA-programmable nuclease is the (CRISPR-associated system) Cas9 endonuclease, for example Cas9 (Csn1) from Streptococcus pyogenes (see, e.g., “Complete genome sequence of an M1 strain of Streptococcus pyogenes.” Ferretti J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference.

Because RNA-programmable nucleases (e.g., Cas9) use RNA:DNA hybridization to determine target DNA cleavage sites, these proteins are able to cleave, in principle, any sequence specified by the guide RNA. Methods of using RNA-programmable nucleases, such as Cas9, for site-specific cleavage (e.g., to modify a genome) are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823-826 (2013); Hwang, W. Y. et al. Efficient genome editing in zebrafish using a CRISPR-Cas system. Nature biotechnology 31, 227-229 (2013); Jinek, M. et al. RNA-programmed genome editing in human cells. eLife 2, e00471 (2013); Dicarlo, J. E. et al. Genome engineering in Saccharomyces cerevisiae using CRISPR-Cas systems. Nucleic acids research (2013); Jiang, W. et al. RNA-guided editing of bacterial genomes using CRISPR-Cas systems. Nature biotechnology 31, 233-239 (2013); the entire contents of each of which are incorporated herein by reference).

The term “recombinase,” as used herein, refers to a site-specific enzyme that mediates the recombination of DNA between recombinase recognition sequences, which results in the excision, integration, inversion, or exchange (e.g., translocation) of DNA fragments between the recombinase recognition sequences. Recombinases can be classified into two distinct families: serine recombinases (e.g., resolvases and invertases) and tyrosine recombinases (e.g., integrases). Examples of serine recombinases include, without limitation, Hin, Gin, Tn3, β-six, CinH, ParA, γδ, Bxb1, φC31, TP901, TG1, φBT1, R4, φRV1, φFC1, MR11, A118, U153, and gp29. Examples of tyrosine recombinases include, without limitation, Cre, FLP, R, Lambda, HK101, HK022, and pSAM2. The serine and tyrosine recombinase names stem from the conserved nucleophilic amino acid residue that the recombinase uses to attack the DNA and which becomes covalently linked to the DNA during strand exchange. Recombinases have numerous applications, including the creation of gene knockouts/knock-ins and gene therapy applications. See, e.g., Brown et al., “Serine recombinases as tools for genome engineering.”Methods. 2011; 53(4):372-9; Hirano et al., “Site-specific recombinases as tools for heterologous gene integration.” Appl. Microbiol. Biotechnol. 2011; 92(2):227-39; Chavez and Calos, “Therapeutic applications of the ΦC31 integrase system.” Curr. Gene Ther. 2011; 11(5):375-81; Turan and Bode, “Site-specific recombinases: from tag-and-target- to tag-and-exchange-based genomic modifications.” FASEB J. 2011; 25(12):4088-107; Venken and Bellen, “Genome-wide manipulations of Drosophila melanogaster with transposons, Flp recombinase, and Φ31 integrase.”Methods Mol. Biol. 2012; 859:203-28; Murphy, “Phage recombinases and their applications.” Adv. Virus Res. 2012; 83:367-414; Zhang et al., “Conditional gene manipulation: Cre-ating a new biological era.” J. Zhejiang Univ. Sci. B. 2012; 13(7):511-24; Karpenshif and Bernstein, “From yeast to mammals: recent advances in genetic control of homologous recombination.” DNA Repair (Amst). 2012; 1; 11(10):781-8; the entire contents of each are hereby incorporated by reference in their entirety. The recombinases provided herein are not meant to be exclusive examples of recombinases that can be used in embodiments of the invention. The methods and compositions of the invention can be expanded by mining databases for new orthogonal recombinases or designing synthetic recombinases with defined DNA specificities (See, e.g., Groth et al., “Phage integrases: biology and applications.” J. Mol. Biol. 2004; 335, 667-678; Gordley et al., “Synthesis of programmable integrases.” Proc. Natl. Acad. Sci. USA. 2009; 106, 5053-5058; the entire contents of each are hereby incorporated by reference in their entirety). Other examples of recombinases that are useful in the methods and compositions described herein are known to those of skill in the art, and any new recombinase that is discovered or generated is expected to be able to be used in the different embodiments of the invention. In some embodiments, the catalytic domains of a recombinase are fused to a nuclease-inactivated RNA-programmable nuclease (e.g., dCas9, or a fragment thereof), such that the recombinase domain does not comprise a nucleic acid binding domain or is unable to bind to a target nucleic acid (e.g., the recombinase domain is engineered such that it does not have specific DNA binding activity). Recombinases lacking DNA binding activity and methods for engineering such are known, and include those described by Klippel et al., “Isolation and characterisation of unusual gin mutants.” EMBO J. 1988; 7: 3983-3989: Burke et al., “Activating mutations of Tn3 resolvase marking interfaces important in recombination catalysis and its regulation. Mol Microbiol. 2004; 51: 937-948; Olorunniji et al., “Synapsis and catalysis by activated Tn3 resolvase mutants.” Nucleic Acids Res. 2008; 36: 7181-7191; Rowland et al., “Regulatory mutations in Sin recombinase support a structure-based model of the synaptosome.”Mol Microbiol. 2009; 74: 282-298; Akopian et al., “Chimeric recombinases with designed DNA sequence recognition.” Proc Natl Acad Sci USA. 2003; 100: 8688-8691; Gordley et al., “Evolution of programmable zinc finger-recombinases with activity in human cells. J Mol Biol. 2007; 367: 802-813; Gordley et al., “Synthesis of programmable integrases.”Proc Natl Acad Sci USA. 2009; 106: 5053-5058; Arnold et al., “Mutants of Tn3 resolvase which do not require accessory binding sites for recombination activity.” EMBO J. 1999; 18: 1407-1414; Gaj et al., “Structure-guided reprogramming of serine recombinase DNA sequence specificity.” Proc Natl Acad Sci USA. 2011; 108(2):498-503; and Proudfoot et al., “Zinc finger recombinases with adaptable DNA sequence specificity.” PLoS One. 2011; 6(4):e19537; the entire contents of each are hereby incorporated by reference. For example, serine recombinases of the resolvase-invertase group, e.g., Tn3 and γδ resolvases and the Hin and Gin invertases, have modular structures with autonomous catalytic and DNA-binding domains (See, e.g., Grindley et al., “Mechanism of site-specific recombination.”Ann Rev Biochem. 2006; 75: 567-605, the entire contents of which are incorporated by reference). The catalytic domains of these recombinases are thus amenable to being recombined with nuclease-inactivated RNA-programmable nucleases (e.g., dCas9, or a fragment thereof) as described herein, e.g., following the isolation of ‘activated’ recombinase mutants which do not require any accessory factors (e.g., DNA binding activities) (See, e.g., Klippel et al., “Isolation and characterisation of unusual gin mutants.” EMBO J. 1988; 7: 3983-3989: Burke et al., “Activating mutations of Tn3 resolvase marking interfaces important in recombination catalysis and its regulation. Mol Microbiol. 2004; 51: 937-948; Olorunniji et al., “Synapsis and catalysis by activated Tn3 resolvase mutants.” Nucleic Acids Res. 2008; 36: 7181-7191; Rowland et al., “Regulatory mutations in Sin recombinase support a structure-based model of the synaptosome.”Mol Microbiol. 2009; 74: 282-298; Akopian et al., “Chimeric recombinases with designed DNA sequence recognition.” Proc Natl Acad Sci USA. 2003; 100: 8688-8691). Additionally, many other natural serine recombinases having an N-terminal catalytic domain and a C-terminal DNA binding domain are known (e.g., phiC31 integrase, TnpX transposase, IS607 transposase), and their catalytic domains can be co-opted to engineer programmable site-specific recombinases as described herein (See, e.g., Smith et al., “Diversity in the serine recombinases.”Mol Microbiol. 2002; 44: 299-307, the entire contents of which are incorporated by reference). Similarly, the core catalytic domains of tyrosine recombinases (e.g., Cre, λ integrase) are known, and can be similarly co-opted to engineer programmable site-specific recombinases as described herein (See, e.g., Guo et al., “Structure of Cre recombinase complexed with DNA in a site-specific recombination synapse.” Nature. 1997; 389:40-46; Hartung et al., “Cre mutants with altered DNA binding properties.” J Biol Chem 1998; 273:22884-22891; Shaikh et al., “Chimeras of the Flp and Cre recombinases: Tests of the mode of cleavage by Flp and Cre. J Mol Biol. 2000; 302:27-48; Rongrong et al., “Effect of deletion mutation on the recombination activity of Cre recombinase.”Acta Biochim Pol. 2005; 52:541-544; Kilbride et al., “Determinants of product topology in a hybrid Cre-Tn3 resolvase site-specific recombination system.” J Mol Biol. 2006; 355:185-195; Warren et al., “A chimeric cre recombinase with regulated directionality.” Proc Natl Acad Sci USA. 2008 105:18278-18283; Van Duyne, “Teaching Cre to follow directions.” Proc Natl Acad Sci USA. 2009 Jan. 6; 106(1):4-5; Numrych et al., “A comparison of the effects of single-base and triple-base changes in the integrase arm-type binding sites on the site-specific recombination of bacteriophage λ.” Nucleic Acids Res. 1990; 18:3953-3959; Tirumalai et al., “The recognition of core-type DNA sites by λ integrase.” J Mol Biol. 1998; 279:513-527; Aihara et al., “A conformational switch controls the DNA cleavage activity of λ integrase.” Mol Cell. 2003; 12:187-198; Biswas et al., “A structural basis for allosteric control of DNA recombination by λ integrase.” Nature. 2005; 435:1059-1066; and Warren et al., “Mutations in the amino-terminal domain of λ-integrase have differential effects on integrative and excisive recombination.” Mol Microbiol. 2005; 55:1104-1112; the entire contents of each are incorporated by reference).

The term “recombine,” or “recombination,” in the context of a nucleic acid modification (e.g., a genomic modification), is used to refer to the process by which two or more nucleic acid molecules, or two or more regions of a single nucleic acid molecule, are modified by the action of a recombinase protein (e.g., an inventive recombinase fusion protein provided herein). Recombination can result in, inter alia, the insertion, inversion, excision or translocation of nucleic acids, e.g., in or between one or more nucleic acid molecules.

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a non-human mammal including but not limited to, a sheep, a goat, horse, a cow, deer, antelope, buffalo, rabbit, camel, alpaca, llama, a cat, rat, mouse, guinea pig, or a dog. In some embodiments, the subject is a non-mammalian vertebrate such as a bird (including turkey, goose, duck, pheasant, quail, grouse, ostrich, emu or pigeon), an amphibian, a reptile, or a fish. In alternative embodiments, the subject is a non-vertebrate animal including but not limited to an insect, crustacean, arachnid, or bivalve. Common examples of non-vertebrate animals include a fly, shrimp, spider, crab, lobster, oyster, clam or a nematode. In some embodiments, the subject is a research animal. In some embodiments, the subject is genetically engineered, e.g., a genetically engineered non-human subject. The subject may be of either sex and at any stage of development.

The terms “target nucleic acid,” and “target genome,” as used herein in the context of transposase, refer to a nucleic acid molecule or a genome, respectively, that comprises at least one target site of a given transposase. In the context of fusions comprising a (nuclease-inactivated) RNA-programmable nuclease and a transposase domain, a “target nucleic acid” and a “target genome” refers to one or more nucleic acid molecule(s), or a genome, respectively, that comprises at least one target site. In some embodiments, the target nucleic acid(s) comprises at least two, at least three, or at least four target sites. In certain preferred embodiments, the target genome comprises a bacterial genome.

As used herein, the term “bacteria” encompasses both prokaryotic organisms and archaea present in microbiota of a subject or occurring in a natural environment not necessarily within a subject.

The terms “intestinal microbiota”, “gut flora”, and “gastrointestinal microbiota” are used interchangeably to refer to bacteria in the digestive tract.

The term “Eubacteria” refers to all bacteria and excludes archaea. In mammals, >90% of all colonic bacteria are in the phyla Firmicutes or Bacteroidetes (Ley et al., Nat Rev Microbiol 2008; 6:776-88).

The term “cecal microbiota” refers to microbiota derived from cecum, which in mammals is the beginning region of the large intestine in the form of a pouch connecting the ileum with the ascending colon of the large intestine; it is separated from the ileum by the ileocecal valve (ICV), and joins the colon at the cecocolic junction.

The term “ileal microbiota” refers to microbiota derived from ileum, which in mammals is the final section of the small intestine and follows the duodenum and jejunum; ileum is separated from the cecum by the ileocecal valve (ICV).

As used herein, the term “probiotic” refers to a substantially pure bacteria (i.e., a single isolate), or a mixture of desired bacteria, and may also include any additional components that can be administered to a mammal for restoring microbiota. Such compositions are also referred to herein as a “bacterial inoculant.” Probiotics or bacterial inoculant compositions of the invention are preferably administered with a buffering agent to allow the bacteria to survive in the acidic environment of the stomach, i.e., to resist low pH and to grow in the intestinal environment. Such buffering agents include sodium bicarbonate, milk, yogurt, infant formula, and other dairy products.

As used herein, the term “prebiotic” refers to an agent that increases the number and/or activity of one or more desired bacteria. Non-limiting examples of prebiotics useful in the methods of the present invention include fructooligosaccharides (e.g., oligofructose, inulin, inulin-type fructans), galactooligosaccharides, amino acids, alcohols, and mixtures thereof. See, e.g., Ramirez-Farias et al., Br J Nutr (2008) 4:1-10; Pool-Zobel and Sauer, J Nutr (2007), 137:2580 S-2584S.

As used herein, the term “metagenomic” refers to the study of genetic material obtained from a defined environment.

As used herein, the term “microbiome” refers to the collection of genomes of microbiota in a defined environment.

As used herein, the term “microbiota” refers to the specific microorganisms present in a defined environment.

Suitable samples for detecting or determining the presence or level of at least one bacterial strain, or for adaptation to a probiotic, typically include whole blood, plasma, serum, saliva, urine, stool (i.e., feces), tears, and any other bodily fluid, or a tissue sample (i.e., biopsy) such as a small intestine or colon sample. Preferably, the sample is serum, whole blood, plasma, stool, urine, or a tissue biopsy.

In addition to the above methods, approaches are disclosed herein to isolate undomesticated but genetically tractable microbes and new mobile plasmids from natural environments, using the mammalian gut and respiratory microbiomes as testbeds. Although microbes are found everywhere in nature, very few can be cultivated in the laboratory and even fewer are genetically tractable. Many microbes are genetically intractable due to various factors, including barriers for DNA entry and active defense mechanisms such as restriction and CRISPR Cas systems. Currently, there are no technologies to address this challenge [Stewart, E. J. Growing unculturable bacteria. Journal of Bacteriology 194, 4151-4160 (2012).]. In particular, a platform technology has been developed that allows for “genome engineering in situ” that involves modifying horizontal gene transfer (HGT) and transposon systems to tag and enrich genetically tractable organisms from a complex microbiota that can serve as new microbial chassis for synthetic biology. Strategies using engineered HGT are inspired by natural HGT processes that occur in many microbial communities, including the gut microbiome, where mobile plasmids and transposons propagate by lateral dissemination [Little, A. E., Robinson, C. J., Peterson, S. B., Raffa, K. F. & Handelsman, J. Rules of Engagement: Interspecies Interactions that Regulate Microbial Communities. Annu Rev Microbiol 62, 375-401 (2008).].

As described herein, the present disclosure successfully demonstrates i) the development of engineered mobile genetic elements to infiltrate and deliver genetic constructs into undomesticated members of a complex microbial community in situ—in the native environment, ii) high-throughput phenotypic selection or screening to identify microbes that are receptive to engineered HGT, iii) implementation of this platform to isolate genetically tractable but undomesticated or unculturable microbes from the natural gut and respiratory microbiomes, and iv) isolation of novel mobile plasmids from natural microbiomes.

Over the past two decades, advances in high-throughput (HT) sequencing to survey microbial populations have greatly outpaced our ability to genetically manipulate microbes. For example, the human gastrointestinal tract is colonized by thousands of bacterial species that are vital for host subject development and homeostasis. However, only a small subset of these bacterial genomes can be manipulated, rendering the rest inaccessible to detailed genetic studies and engineering.

According to certain method embodiments, disclosed are two platform approaches, herein referred to as Genome Tagging and Retrieval In Situ (GTRIS) and Plasmid Trap and Retrieval In Situ (PTRIS) that involve engineering conjugative transposon systems that can be implemented in a controlled fashion in the native environment of undomesticated microbes to deliver user-defined genetic circuits to engineer the target microbe and capture new mobile plasmids. Studies are provided herein that demonstrate these new capabilities by genetically manipulating an unculturable gut microbe, Segmented Filamentous Bacteria (SFB), and other gut and respiratory microbiota, as well as isolating new and useful mobile plasmids from those communities. Current technological limitations prevent genetic tools from being implemented in complex natural environments, where many useful microbes have yet to be (or cannot be) cultivated in the laboratory. The development of genetic tools for undomesticated microbes will significantly expand our currently limited repertoire of microbial chasses for engineering new functions into cells that live in complex and challenging environments. Here, quantitative metrics for success include the number of new microbes that can be engineered and new plasmids that can be captured. This generalized platform can be implemented for a broad array of useful applications spanning from the human microbiome to animal, soil, or other surface-associated microbiomes, such as biofilms on ship hulls or open wounds.

Provided below is an overview of some general examples related to the Development and Implementation of Embodiments of the invention. Note that different acronyms are set forth, which provide a shorthand for certain embodiments. Such acronyms are helpful to describe certain implementations of a given embodiment, and are not intended to be limiting.

### Example 1: Engineering a Programmable Host-Range Conjugative Transposon System to Infiltrate a Microbial Community In Situ

A general delivery system using E. coli as a donor vehicle has been developed to infiltrate a native microbiome, transmit a genetic payload stably into the genomes of recipient microbes, and isolate successful recipients via HT enrichment or selection. This donor can be used to genetically manipulate a number of different bacteria, including Proteobacteria, Firmicutes, and Bacteroidetes, which constitute important microbes for industrial and medical biotechnology.

Strain engineering: “Shuttle strains” have been engineered that act as intermediary microbial hosts for mobile conjugative plasmids. To transmit the mobile plasmids into both gram+ and gram− microbes, a probiotic E. coli strain has been utilized, which is naturally adapted to mammalian associated environments. In a specific example, the E. coli Nissle strain has been engineered to contain chromosomal KO of the asd gene, which causes metabolic auxotrophy for diaminopimelic acid (DAP), an essential cell wall component [Pansegrau, W. et al. Complete nucleotide sequence of Birmingham IncP alpha plasmids. Compilation and comparative analysis. J Mol Biol 239, 623-663 (1994)]. In the absence of DAP supplementation, the Δasd strain is unable to grow, which provides a control of strain growth in complex environment such as the mammalian GI or respiratory tract. In addition, the strain is chromosomally tagged with a red-fluorescence protein (RFP) marker to distinguish it from natural microflora for downstream identification.

Conjugation engineering: Self-transmissible mobile genetic elements are prevalent in nature. Certain embodiments involve engineering the RK2/RP4 promiscuous conjugative plasmid, which is a member of the IncP-α family of broad-host range plasmids [Lampe, D. J., Akerley, B. J., Rubin, E. J., Mekalanos, J. J. & Robertson, H. M. Hyperactive transposase mutants of the Himar1 mariner transposon. Proc. Natl. Acad. Sci. U. S. A. 96, 11428-33 (1999).]. The 60kb RK2 plasmid encodes over 70 genes necessary for its own independent replication and mobilization via pilus-based transfer, and appears to stably propagate in many Gram+/− bacteria even in the absence of selection. It has been discovered that the RK2 system is highly competent and able to mobilize between E. coli strains at 90-100% efficiency on solid medium. In particular, the wild-type RK2 plasmid has been engineered and implemented as the basis of conjugation system of many embodiments described herein.

In certain embodiments, the three natural antibiotic resistance genes (cat, bla, kan) were removed from RK2 and, optionally, a mobile transposon system was introduced into a permissive location in the RK2 plasmid. Accordingly, examples of mobile plasmids contain the origin of transfer (oriT) recognition sequences that allows trans-conjugation by RK2. These plasmids can be stably maintained in a variety of microbes. Upon mating of the donor cell with the target recipient, the engineered transconjugant plasmid is transferred into the recipient, where an engineered transposon can integrate into the genome to increase stability.

Transposon engineering: In one example, the Himar transposon, a hyperactive variant of the mariner transposon [Lee, S. M. et al. Bacterial colonization factors control specificity and stability of the gut microbiota. Nature 501, 426-429 (2013)] is implemented into a integrative plasmid vector. Himar integrates nonspecifically at T/A base-pairs and does not require specific host factors for transposition. As T/A bases tend to be located in non-coding regions, Himar transposons are less likely to disrupt genes. To avoid natural allosteric inhibition of transposition at high transposase levels, the Himar1C9 variant can be used, which is insensitive to Himar protein levels [Yaung, S. J. et al. Improving microbial fitness in the mammalian gut by in vivo temporal functional metagenomics. Mol. Syst. Biol. 11, 788 (2015)]. In a specific embodiment, the Himar transposon is engineered to carry a selectable gene payload sequence flanked by a modified Himar1 inverted repeat (IR) sequences (TAGACCGGGACTTATCATCCAACCTGT; SEQ ID NO: 12). The modified Himar1 IR sequence contains a MmeI Type IIS restriction enzyme recognition site (underlined) which cuts 20-21 bps away from the recognition site, leaving ˜17 bps of flanking genomic DNA. This enables deep sequencing to determine the genomic insertion loci of the payload gene. The combination of Himar mutagenesis and deep-sequence, dubbed Tn-seq, has been applied to several microbes [Klümper, U. et al. Broad host range plasmids can invade an unexpectedly diverse fraction of a soil bacterial community. ISME J. 9, 934-945 (2015]. In the event that the Δasd auxotrophy does not limit growth of the engineered shuttle cells in vivo, additional auxotrophic markers are deployed. Data provided herein shows that a series of engineered shuttle strains are capable of RK2 conjugation and Himar transposition into any targeted microbial community.

### Example 2: Development of a Broad-Host Range Gene Expression System to Activate Genetic Elements in a Variety of Microbes.

In the GTRIS system, foreign DNA is transferred from donors into recipients via RK2-mediated conjugation. Upon transfer, the foreign DNA must have sufficient gene expression of the transposase to insert the payload gene into the recipient genome. The payload must then have sufficient expression of its marker tag for phenotypic selection. Here, we aim to generate regulatory elements for a broad range of microbial recipients.

Design of constitutive regulatory elements: An HT reporter system may be implemented to measure transcriptional and translational activity of thousands of cis-regulatory elements in a variety of microbes. The system uses microarray oligo synthesis to generate large promoter/RBS libraries, and RNAseq and FACSseq to measure transcription and translation levels. We have characterized gene expression activity of ˜30,000 promoters/RBSs from ˜180 phylogenetically diverse microbes in E. coli, B. subtilis, and P. aeruginosa. Based on this experimental dataset and available genome sequences, this enabled development of algorithms to predict regulatory elements that are active in various microbial clades. Regulatory regions are identified from essential genes in ˜15,000 sequenced genomes in NCBI, clustered by phylogeny, and used to perform motif discovery on these regulatory clusters to find DNA motifs that promote transcriptional and translational activity. These motifs can be mapped to measured activity levels from our experimental library to tune algorithm parameters.

Engineering host-specific regulatory elements: In one example, RNAi/CRISPRi-based strategies are implemented to reduce cross-expression of the payload construct and transposase in donor cells. One approach is to express synthetic small regulatory RNAs (sRNAs) for gene knockdown in donors. The sRNAs consist of a target-binding sequence that binds a specific complementary mRNA, and a scaffold that recruits RNA-binding proteins to block translation. sRNAs can be designed to knockdown one or more mRNAs in vivo [Yoo, S. M., D. Na, and S. Y. Lee, Design and use of synthetic regulatory small RNAs to control gene expression in Escherichia coli. Nat Protoc, 2013. 8(9): p. 1694-707]. A second approach uses CRISPR interference (CRISPRi) [Larson, M.H., et al., CRISPR interference (CRISPRi) for sequence-specific control of gene expression. Nat Protoc, 2013. 8(11): p. 2180-96], which prevents transcription of the target gene or genes. CRISPRi uses the natural CRISPR system, in which small RNAs are targeted to specific DNA sequences and recruit the Cas9 endonuclease to cleave the DNA. In CRISPRi, a catalytically-inactivated Cas9 is expressed in the donor strain and recruited by synthetic small guide RNAs (sgRNA) to block transcription of a target gene. By respectively inhibiting translation and transcription, sRNAs and CRISPRi are two orthogonal strategies that can be used in combination to regulate gene expression. To ensure these systems are active only in donor cells, sRNA and sgRNA/Cas9 sequences can be genomically integrated or placed on a non-conjugative plasmid.

To minimize technical risks, certain embodiments employ engineering regulatory elements only for Proteobacteria, Firmicutes, and Bacteroidetes, and adopting HT synthesis and assays to test thousands of constructs simultaneously. Additionally, the dual sRNA/CRISPRi approach will reduce leaky expression and limit toxicity. These approaches deliver a library of regulatory elements active in different microbes and a design algorithm to generate additional elements. The resulting expression system and parts list enables programmatic gene activation in phylogenetically diverse microbial clades from 3 major phyla.

### Example 3: Implementation of a HT Selection Platform to Directly Isolate Genetically Tractable Microbes from Complex Communities.

Genetically manipulatable microbes in a complex community will have a higher likelihood to receive and express the targeted payload. One embodiment utilizes HT selection methods of Fluorescence Activated Cell Sorting (FACS) and antibiotic resistance (AR) to isolate these engineerable recipients that heterologously express the foreign payload tag and identify the resulting strains by 16S rRNA for downstream cultivation.

FACS enrichment: The transposon system developed in Strategy 1 was engineered to contain a constitutively active fluorescence gene cassette (expressed by promoters from Example 2) that produced a signal detectable by flow cytometry in recipient cells where conjugation and transposition have occurred. The super-folding green fluorescence protein (sfGFP) was implemented for aerobic microbes and a flavin-based fluorescence protein (Fbfp) was implemented for anaerobic microbes [Drepper, T., et al., Flavin mononucleotide-based fluorescent reporter proteins outperform green fluorescent protein-like proteins as quantitative in vivo real-time reporters. Appl Environ Microbiol, 2010. 76(17): p. 5990-4]. A FACS protocol was developed (see Examples Section) to select against the Red channel and for the Green channel. In a mixed culture, the donor is RFP+/GFP-, while transconjugant recipients are RFP−/GFP+. Multiple rounds of FACS may be needed to remove residual donor cells. FACS-enriched recipients are then identified by 16S and also grown on various rich media to culture isolates.

Antibiotic resistance enrichment: Concurrent to FACS approach, the detection limit of transconjugation is further reduced by using AR selection, which can be applied cheaply to cell populations of >1010. The antibiotic resistance profile of the native recipient microbiome is first characterized to select the best resistance marker to use in our transposon. Preliminary studies suggested that the murine gut microbiota has very limited resistance (<10 CFU/gram fecal mass) to chloramphenicol and tetracycline. Other recent works have determined AR profiles of human fecal microbiota [Sommer, M.O., G. Dantas, and G. M. Church, Functional characterization of the antibiotic resistance reservoir in the human microflora. Science, 2009. 325(5944): p. 1128-31]. In addition to bulk 16S profiling of recipient cells, selection protocols are implemented for isolating AR-tagged recipients from AR-sensitive donor strains.

In the event that FACS enrichment has high levels of background due to residual donor cells, the enrichment protocol can be implemented over multiple iterations to reduce background. Using multiple antibiotic selections mitigates technical risks associated with pan-resistance of the recipient microbiota. MIC levels can also be determined to ensure proper antibiotic dosage. These experiments are expected to deliver a HT enrichment protocol to isolate cells that contain one of four screenable/selectable cassettes (GFP, FbfP, two AR) for horizontal transfer using the engineered conjugation and transposon system developed in Thrust 1.

### Example 4: Isolation of Novel Genetically Manipulable Microbes from In Vitro Communities and In Vivo Environments in Live Animals

The Genome Tagging and Retrieval (GTR) system embodiment is implemented in microbiota populations both in vitro in laboratory conditions and in situ in the native mammalian environment, in order to isolate new microbial chasses that are amenable to genetic manipulation. In vitro GTR is implementable in synthetic communities and mammalian-associated microbiota and in situ GTR in murine models that are colonized with an unculturable gut microbe or with a complex community of natural microflora.

In vitro GTR of synthetic consortia: E. coli shuttle strain described above with respect to Strategy 1 that contains the RK2-Himar conjugation-transposon system is introduced to a synthetic community of eight nonpathogenic microbes (E. coli, S. enterica, P. aeruginosa, B. subtilis, L. reuteri, B. thetaiotaomicron, E. faecalis, and V. cholera). To assess rates of conjugation and transposition, the FACS/AR enrichment protocol described under Strategy 3 above was used. Based on preliminary results, high transfer and capture rates (>40% efficiency) for E. coli, S. enterica, E. faecalis and moderate rates for L. reuteri, B. thetaiotaomicron, V. cholera, and P. aeruginosa were observed. 16S genotyping of GFP+/RFP− cells from the resulting population yields the distribution of transfer efficiencies for each of the 8 species in comparison to the starting population.

GTR of gut microbiota: To demonstrate GTR in a complex natural microbiota, a transposon payload was introduced to intestinal microbes by in vitro conjugation of the E. coli shuttle strain with fresh murine fecal samples from conventionally-raised B6 mice. Donor concentration and mating time affect trans-conjugation and transposition efficiency can be determined. FACS/AR enrichment may be applied to the fecal population and GFP+/RFP− or AR+ cells will be isolated and identified by 16S. These resulting microbes may be cultivated in a 3:2PAS medium for culturing a significant fraction of gut microbiota. To demonstrate GTR in situ, the Δasd E. coli shuttle strain harboring RK2 and a plasmid library of pJN105, pFD340, pJP028 with the Himar-(GFP/AR) transposon is introduced into the murine gastrointestinal (GI) tract. The shuttle strain is introduced into conventional B6 mice by oral gavage and allowed to equilibrate for 24-hours. Fecal samples are assessed for conjugation and transposition into the native gut microbiota by FACS/AR enrichment. Effectiveness of the system may be demonstrated by applying it to mice that are mono-associated with Segmented Filamentous Bacteria (SFB), a thus-far unculturable but sequenced gut microbe that immunologically interacts with the host subject intestine [Ivanov, II, et al., Induction of intestinal Th17 cells by segmented filamentous bacteria. Cell, 2009. 139(3): p. 485-98]. Successful introduction of Himar transposons to SFB in situ enables Tn-seq to interrogate the role of various SFB genes in intestinal colonization in vivo.

GTR of bronchoalveolar microbiota: GTR may be implemented in vitro on clinical respiratory microbiota from patients with respiratory distress who undergo bronchoalveolar gavage. These respiratory microbiota tend to contain Pseudomonas, Streptococcus, and Staphylococcus species. Genetic tractability into these pathogenic species can be used to develop therapies to fight against pulmonary bacterial infections, especially for patients with cystic fibrosis and other chronic conditions. Preliminary results show an RK2 conjugation efficiency of >17% into these respiratory microbiota by FACS analysis. GTRIS donors will be introduced to the oropharyngeal cavity in live mice and recipients will be characterized after 24-hour exposure. Success of the GTRIS approach depends at least in part on overcoming several technical challenges. The engineered genetic payload must be delivered into the target microbial community using live donor cells and engineered conjugation machinery. Upon transfer, the genetic payload must evade host defenses through genomic integration. The payload must be expressed in the recipient, resulting in a new identifiable phenotype (e.g. antibiotic resistance or fluorescence). Finally, successful recipients need to be cultivated. Data is provided herein that utilizes RK2 conjugative plasmid and Himar transposon. Other plasmid and transposon systems including pBBR122 [Kovach, M. E., et al., pBBR1MCS: a broad-host-range cloning vector. Biotechniques, 1994. 16(5): p. 800-2] and Tn5 [Reznikoff, W.S., The Tn5 transposon. Annu Rev Microbiol, 1993. 47: p. 945-63] can be used based on the teachings provided herein.

GTRIS has been demonstrated to introduce new genetic cassettes into both in vitro synthetic and in vivo mammalian-associated microbial communities of the gut and respiratory tract. It is demonstrated herein that these genomically tagged microbes can be retrieved from the native community to establish new microbial chasses.

### Example 5: Capture of Natural Mobile Plasmids

To expand the current plasmid repertoire used for different microbes, a plasmid capture technology dubbed Plasmid Trap and Retrieval in situ (PTRIS), which uses “reservoir” cells as recipients of naturally mobilizing plasmids from a complex community is used for isolation by high-throughput selection and detailed characterization [Jones, B. V. and J. R. Marchesi, Transposon-aided capture (TRACA) of plasmids resident in the human gut mobile metagenome. Nat Methods, 2007. 4(1): p. 55-61]. The PTRIS approach is highly synergistic to the GTRIS methodology as the donor GTR shuttle strain can also act as the PTR reservoir strain.

Plasmid capture & selection: GFP-labeled strains (E. coli, P. aeruginosa, L. reuteri, B. thetaiotaomicron) may be implemented as plasmid capturing cells (PCCs) and GTR may be applied to PCCs in complex microbiota communities. As most natural plasmids contain antibiotic resistance genes, a FACS/AR protocols are described herein to retrieve GFP+/AR+ PCCs that have gained new resistance phenotypes indicative of acquisition of new plasmids. A panel of antibiotics may be used to which PCCs are initially sensitive but may become resistant after acquiring a novel plasmid.

PTRIS implementation: PTRIS is demonstrated to capture mobile plasmids from the gut and respiratory murine models described in Strategy 4. Concurrent with GTRIS, the donor cells (GFP+) can be sorted into a separate FACS bin, where the cells are then tested for antibiotic sensitivity. New plasmid transfers can result in new antibiotic resistances in the GFP+ donor cells. PCCs strains can be applied to the murine microbiota both in vitro and in situ to assess the plasmid capture rates under those environmental conditions.

Plasmid characterization: Upon isolation of PCCs that contain new resistances, the novel plasmids may be purified and shot-gun sequencing of PCC isolates can be performedto determine its full sequence.

The function and advantage of these and other embodiments of the present invention will be more fully understood from the Examples below. The following Examples are intended to illustrate the benefits of the present invention and to describe particular embodiments, but are not intended to exemplify the full scope of the invention. Accordingly, it will be understood that the Examples are not meant to limit the scope of the invention.

### Example 6 Metagenomic Alteration of Gut Microbiome by In situ Conjugation (MAGIC)

### Introduction

In nature, microbes live in open, dynamic and complex habitats that are difficult to recapitulate in a laboratory setting. While recent advances in deep sequencing have shed light on the vast microbial diversity in nature, our ability to genetically alter these microbiomes remains limited despite advances in culturomics and synthetic biology¹⁻⁴. Genetic intractability is often attributed to host immunity such as restriction-methylation⁵ or CRISPR-Cas processes⁶, although a myriad of other factors (e.g., DNA transformation, growth state, fitness burden) may also influence gene transfer potential⁷. A specific example of implementation of the embodiments disclosed herein and demonstration of the proof of concept and versatility of the embodiments involves an approach, coined Metagenomic Alteration of Gut microbiome by In situ Conjugation (MAGIC), to genetically modify gut microbiota in their native habitat by engineering the mobilome—the repertoire of mobile genetic elements in the gut microbiome.

MAGIC was applied to the mammalian gut because it harbors a diverse microbial community that plays key functional roles in host physiology⁸. An Escherichia coli donor strain was constructed that can deliver a genetic payload into target recipients by broad host-range bacterial conjugation (FIG. 1). The IncPα-family RP4 conjugation system⁹, which can efficiently conjugate into both Gram-positive and Gram-negative cells, was integrated into the EcGT1 donor genome along with a constitutively expressing mCherry-specR cassette (ΔgalK::mCherry-specR). To strengthen biocontainment of the donor and to facilitate in vitro selection of recipients, an alternative strain EcGT2 (Δasd::mCherry-specR) was generated to be auxotrophic for the essential cell wall component diaminopimelic acid (DAP), thus requiring DAP supplementation in the growth media¹⁰.

### Construction of Modular Mobile Plasmids and In Vitro Validation

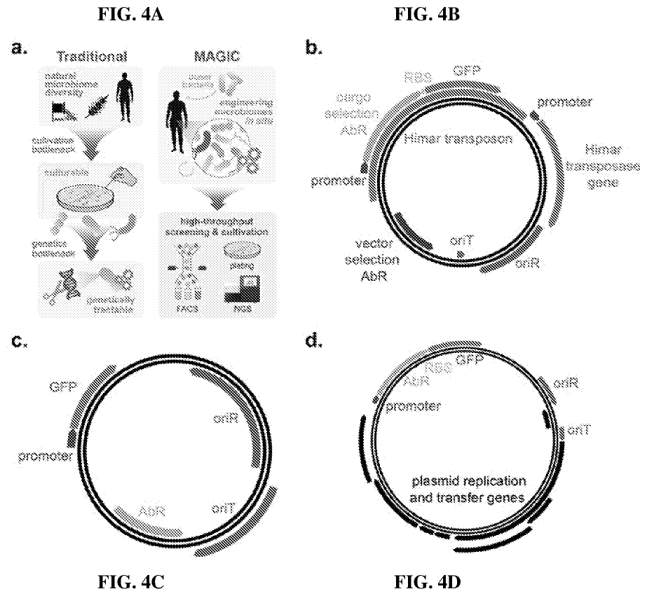

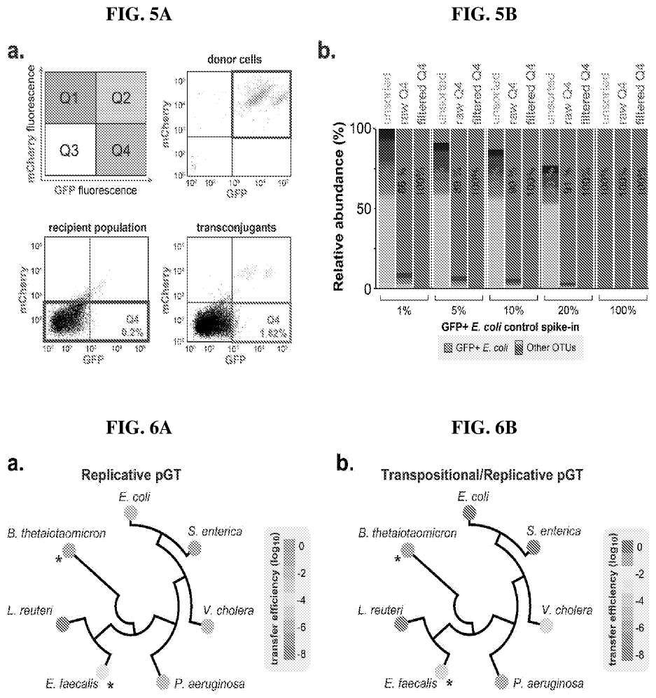

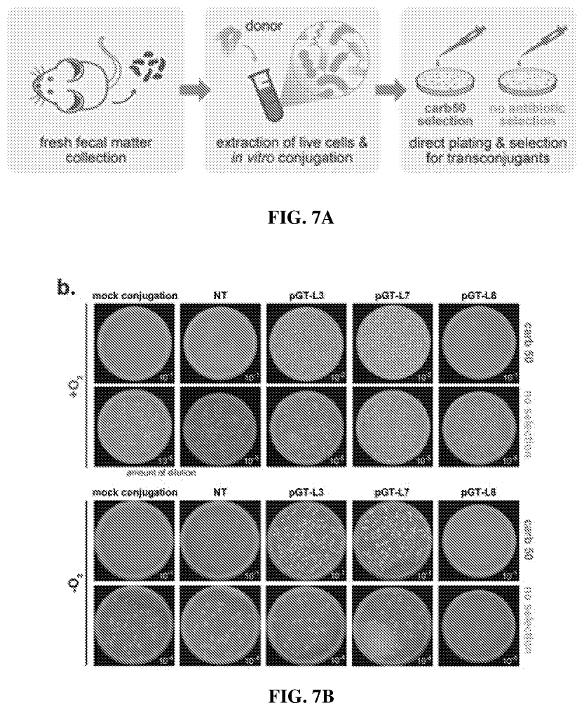

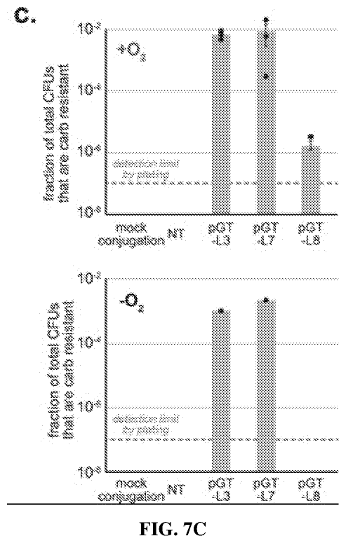

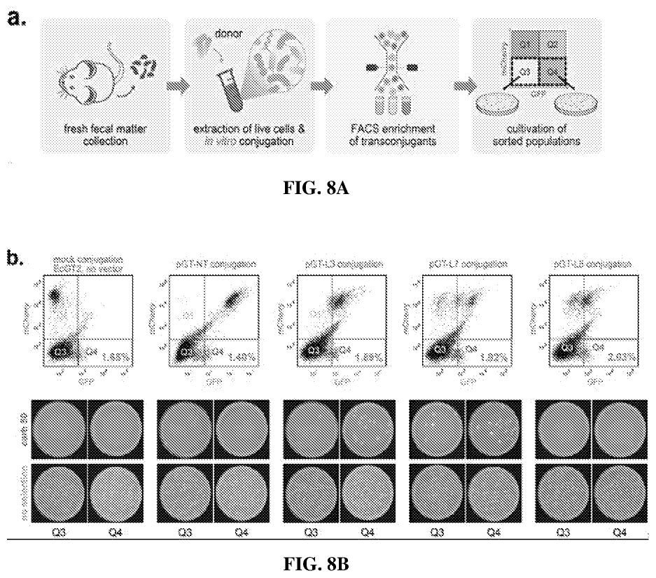

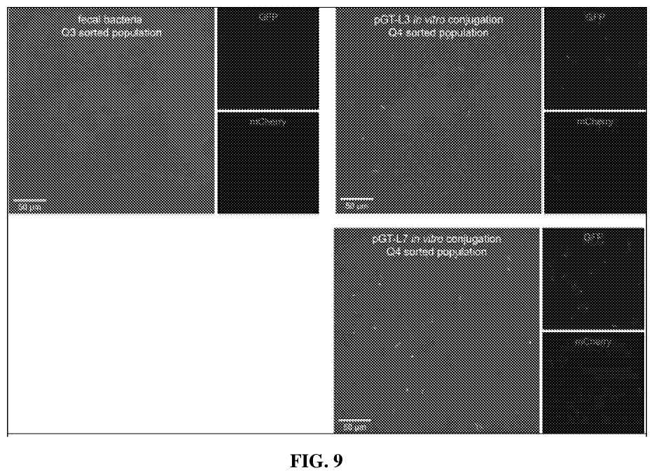

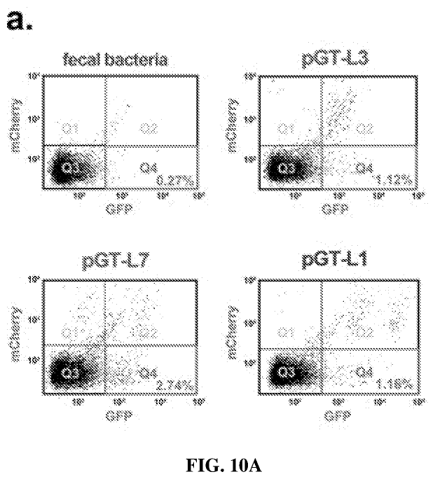

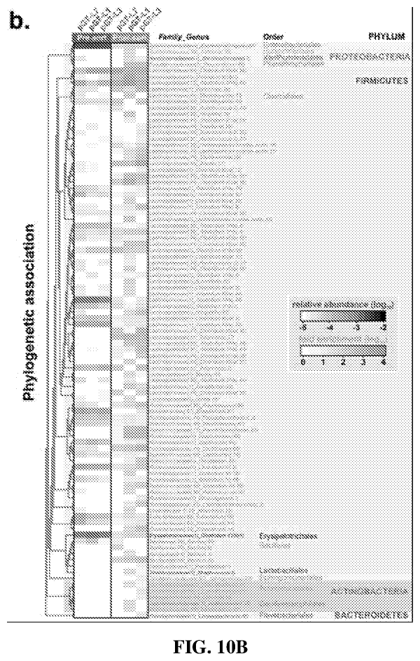

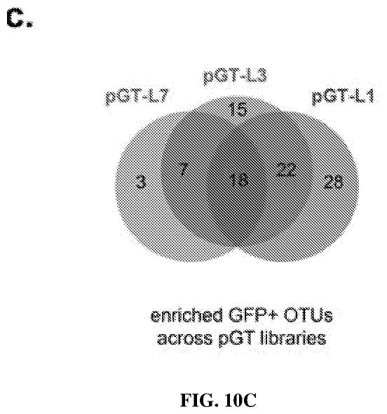

We developed a modular suite of mobile plasmids (pGT) that featured replicative origins with narrow to broad host-ranges, a RP4 transfer origin, a selectable marker, and the desired genetic payload (Tables 1-3, FIG. 4A-FIG. 4D). A broad host-range Himar transposon system was also utilized for delivering integrative payloads. As a demonstration of the system, we used a dual-reporter payload harboring a green fluorescent protein (GFP) and an antibiotic resistance gene (AbR). The use of Fluorescence Activated Cell Sorting (FACS) combined with 16S metagenomic analysis enables identification of successfully modified recipients or transconjugants, which can then be readily isolated on antibiotic selective plates. This multi-pronged strategy can increase the diversity of genetically tractable microbiota that can be captured. We first validated and optimized MAGIC protocols in vitro by assessing the gating stringency of FACS with control spike-ins of GFP-tagged bacteria into a complex sample community (FIG. 5A-FIG. 5B). Subsequently, in vitro conjugations with defined recipient species (FIG. 6A-FIG. 6B) and live bacterial communities extracted from mouse feces (FIG. 7A-FIG. 7C) demonstrated the transfer of the payload from donors to recipients to yield GFP+ transconjugants that could be enriched by FACS (FIG. 8A-FIG. B), which were confirmed by fluorescence microscopy (FIG. 9). 16S rRNA sequencing of FACS-enriched transconjugant populations revealed a diverse range of recipient bacteria (FIG. 10A-FIG. 10C).

### In Situ Conjugation

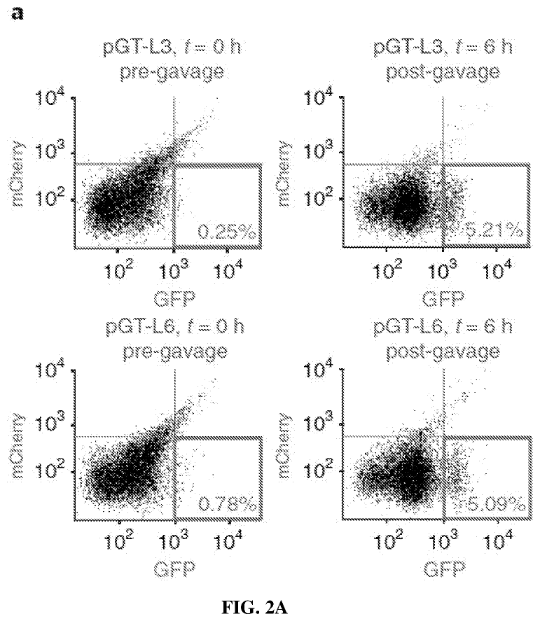

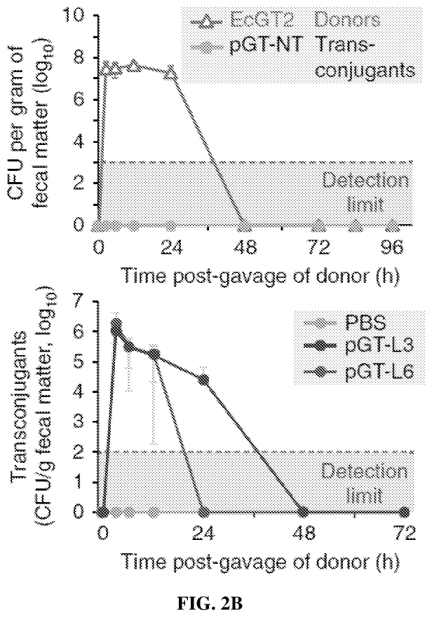

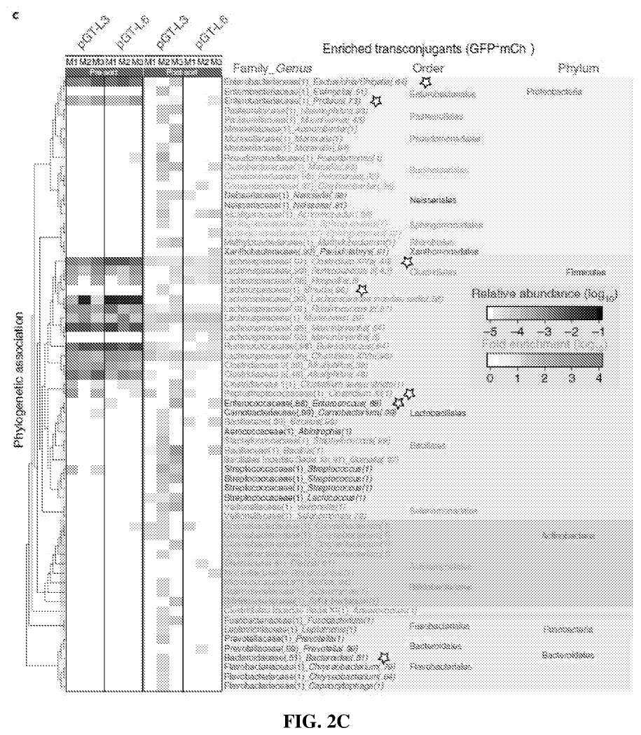

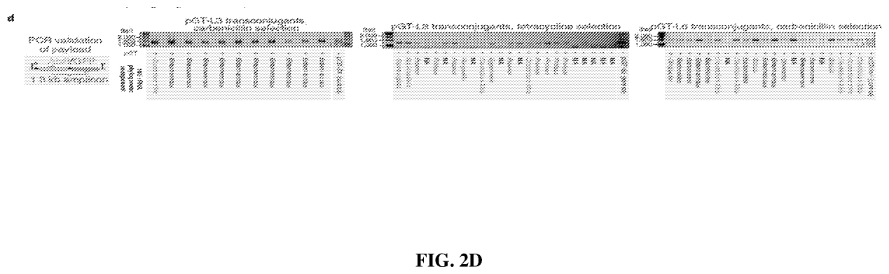

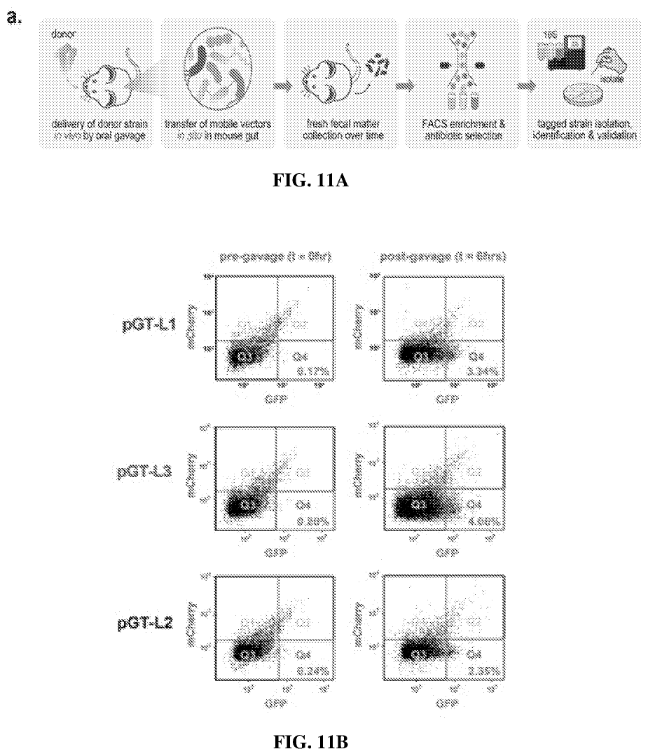

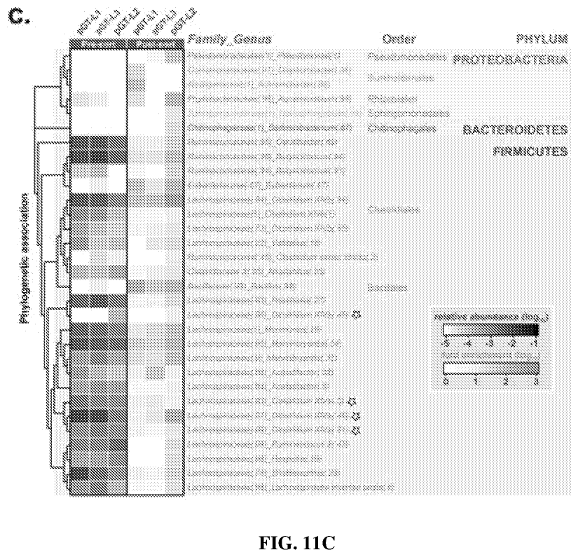

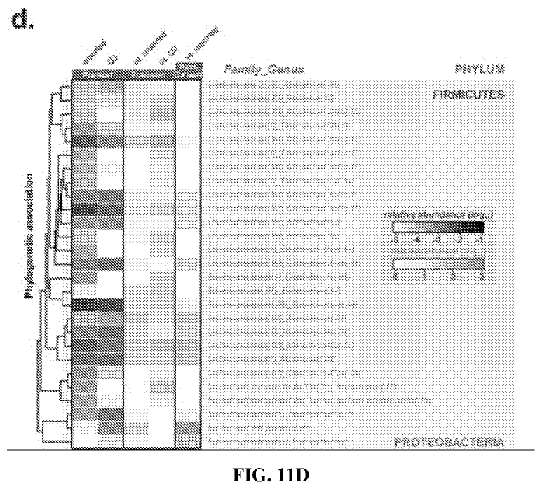

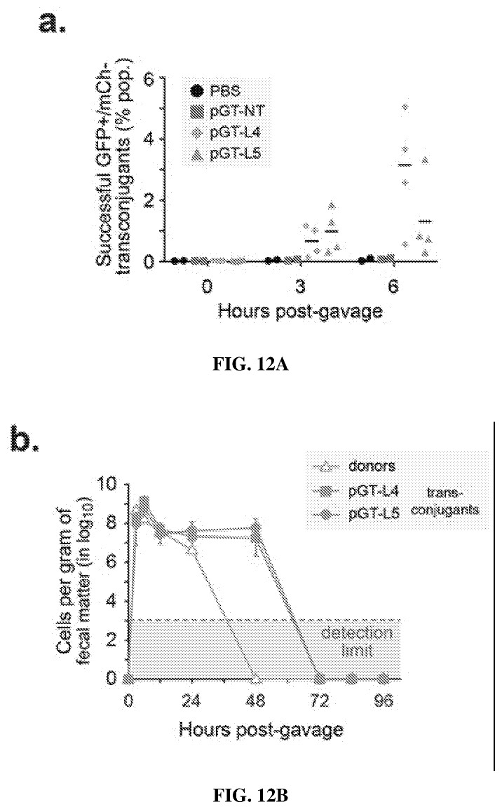

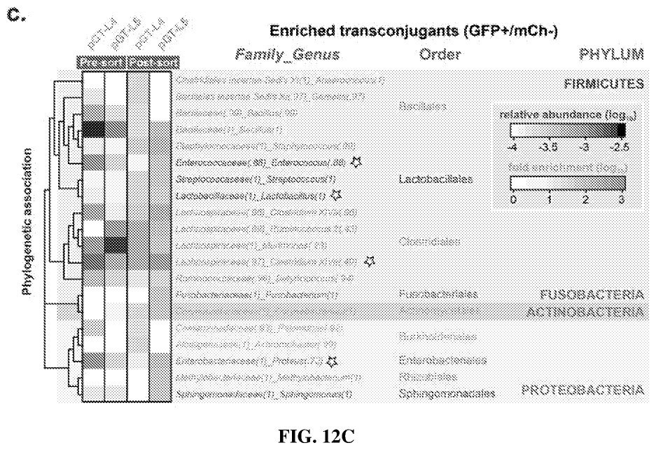

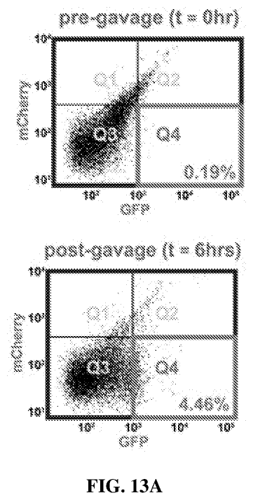

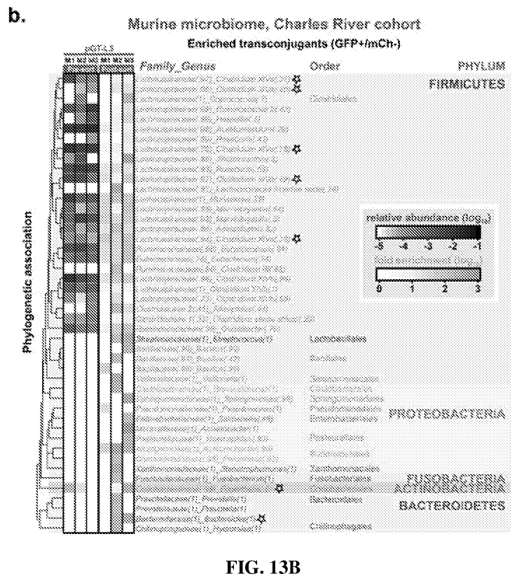

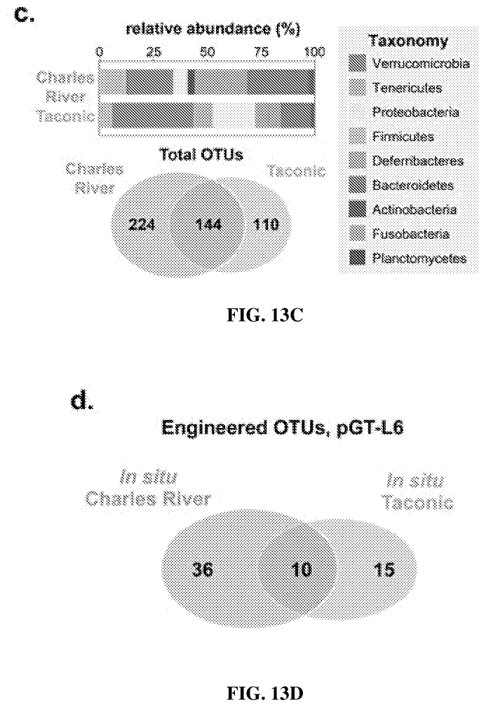

Next, we explored the possibility of implementing MAGIC in vivo, directly in the native gut microbiome of an animal. We hypothesized that different groups of microbiota may be modified by using a library of pGT vectors that exhibit a range of gene expression levels and plasmid replication elements suitable for different gut bacteria. Libraries of pGT vectors (pGT-L1 to pGT-L6) were generated by modularly permuting pGT parts, including regulatory sequences of varying activity, payload selectable genes (bla, catP, tetQ), transposon elements (Himar), and plasmid origins (RSF1010, pBBR1, p15A) (Tables 1 and 2). We carried out 4 separate in vivo studies where EcGT2 donors containing pGT libraries were orally gavaged into conventionally raised C57BL6/J mice obtained from commercial vendors (FIG. 11A). To assess the transfer capacity of individual pGT replicative or integrative designs (pBBR1, p15A-Himar, and RSF1010), we introduced each pGT library (pGT-L1, pGT-L2, or pGT-L3, respectively) separately into a mice cohort from Taconic (FIG. 11B-FIG. 11D). We tested larger combinatorial libraries (pGT-L3 to pGT-L6) in two independent mice cohorts to assess variability across cohorts (FIG. 2A-FIG. 2D, FIG. 12A-FIG. 12C). To compare in situ transfer in different gut communities, we tested the pGT-L6 library in mice from a different source (Charles River) (FIG. 13A-FIG. D).

### Enrichment and Characterization of Transconjugants

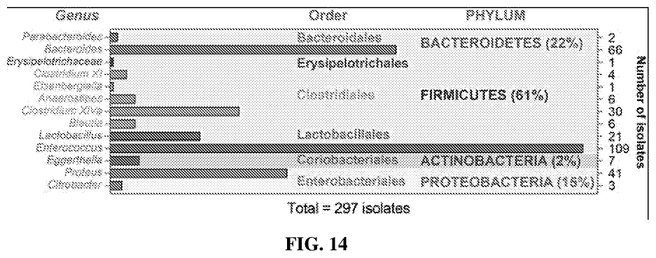

We performed FACS enrichment and 16S metagenomic analysis on fecal material from all mice studies collected over time after oral gavage of pGT libraries. Across in situ studies, up to 5% of resulting bacteria appeared to be successful transconjugants (i.e., GFP⁺/mCherry⁻) six hours post-gavage, compared to control groups (mice gavaged with PBS or EcGT2 carrying a non-transferrable vector pGT-NT) (FIG. 2A, FIG. 11B, FIG. 12 A, FIG. 13A). These GFP⁺/mCherry⁻ transconjugants persisted for up to 72 hours post-gavage (FIG. 2B, FIG. 12B). 16S metagenomic sequencing of these transconjugant populations revealed a wide phylogenetic breadth (FIG. 2C, FIG. 11C, FIG. 12, FIG. 13B). Importantly, we observed significant reproducible enrichment of Proteobacteria and Firmicutes, especially Clostridiales and Bacillales, amongst successful transconjugants across multiple independent experiments. Using the same pGT-L6 library in mice from different vendors, which harbored distinct microbiomes (FIG. 13C), yielded shared and distinct transconjugants (FIG. 13D). In parallel to FACS-metagenomic studies, we isolated individual transconjugants from these fecal samples by selective plating for the payload antibiotic resistance gene and confirmed the presence of the GFP-AbR payload by PCR (FIG. 2D). Across all experiments, we isolated and validated over 297 transconjugants belonging to 19 genera across 4 phyla (FIG. 14, Table 4), validating the capacity of MAGIC to broadly transfer genetic material in situ into diverse recipients in the mammalian gut. In contrast, only 7 genera could be isolated from in vitro conjugation experiments using the same pGT vectors despite comparable diversity of transconjugants detected by FACS-metagenomics (FIG. 10A-FIG. 10C). This difference may be due to in vitro conditions that sub-optimally support growth of diverse species during conjugation reactions, which underscores the value of implementing MAGIC in situ in an established complex microbiome.

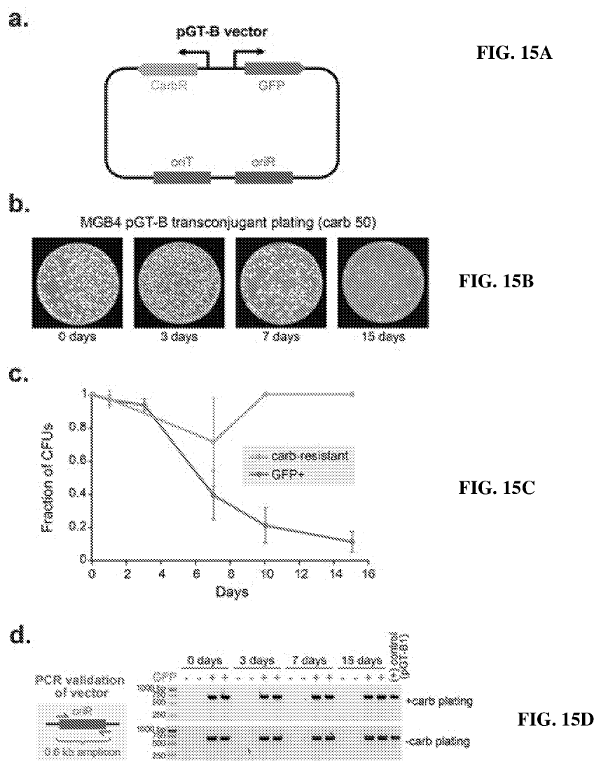

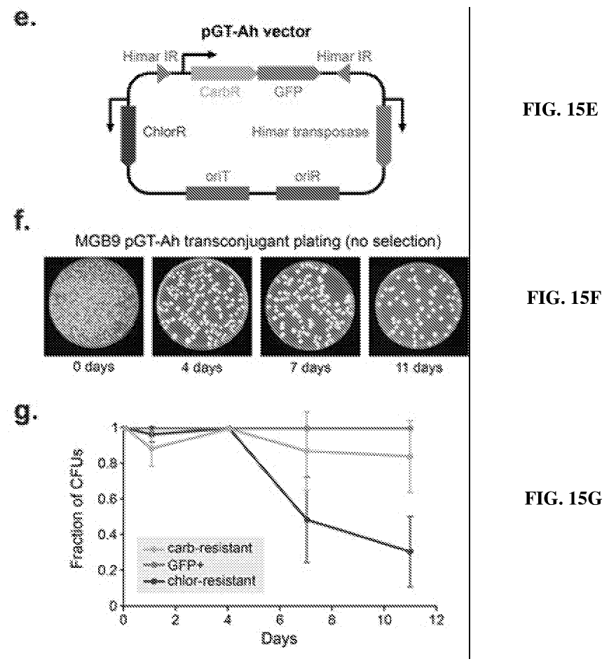

Since transconjugants were no longer detected by 72 hours in situ (FIG. 2B, FIG. 12B), we speculated that the genetic payload (GFP-AbR) on pGT vectors might be unstable or toxic, thus causing its negative selection in transconjugants. This hypothesis was tested in vitro by 20-30 serial passages of two transconjugant isolates of Escherichia fergusonii that contained the GFP-carbR payload either on a pGT-B1 (replicative pBBR1 origin) or a pGT-Ah1 (integrative Himar transposon) plasmid (FIG. 15A- FIG. G). For the pGT-B1 population, we observed a significant increase in the fraction of GFP(-) cells (FIG. 15A-FIG. 15C). PCR assay of the origin of replication indicated that the pGT-B1 plasmid was no longer present in these GFP(−) cells (FIG. 15D). In contrast, cells in the pGT-Ah1 population remained GFP(+) despite a detectable loss of the plasmid in parts of the population over time (FIG. 15E-15G), which suggests a more stable maintenance of the GFP-CarbR payload as a integrative transposon within the host genome. Together, these results highlight the challenges of maintaining long-term in vivo stability of engineered genetic constructs in complex microbial communities, and suggest design considerations for more precise tuning of payload life-span and for improving payload biocontainment.

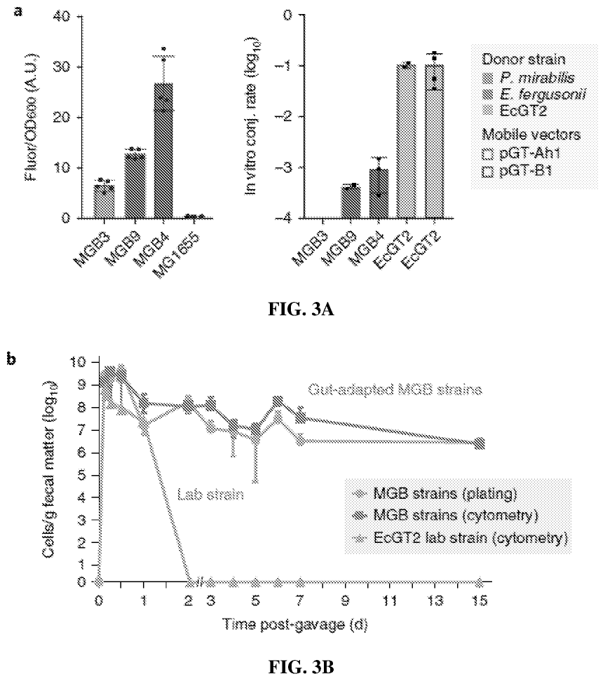

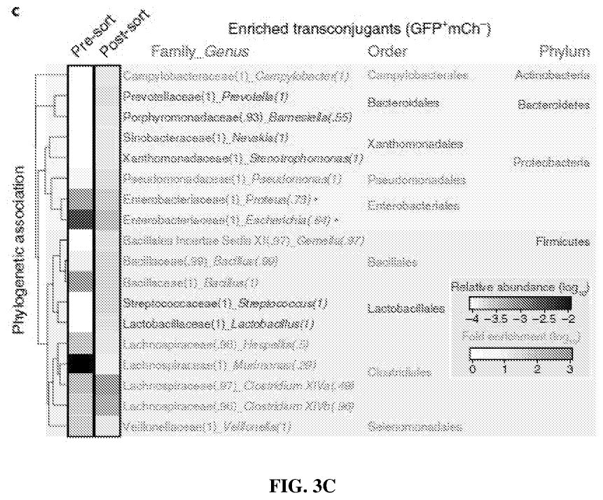

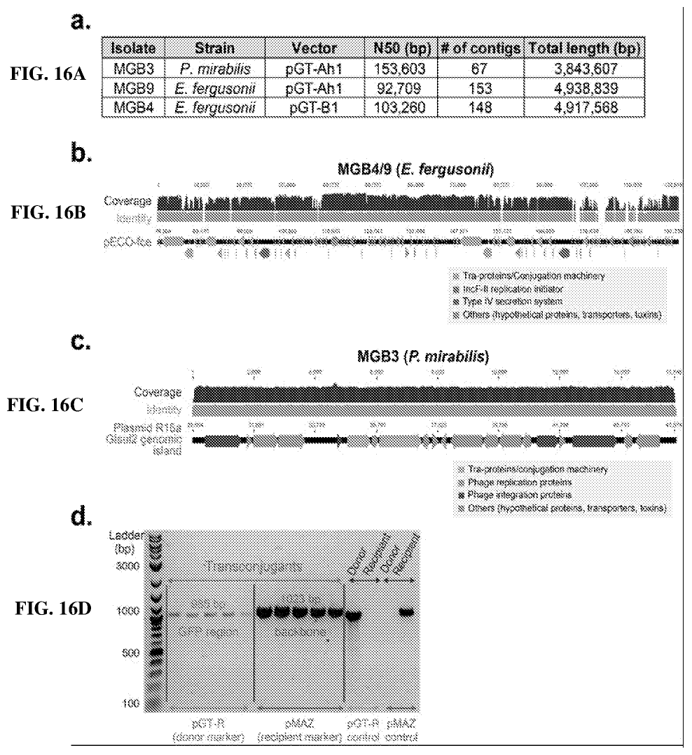

Whole genome sequencing of three transconjugant strains of Proteus mirabilis and Escherichia fergusonii from our studies (designated as Modifiable Gut Bacteria MGB3, MGB4, and MBG9) revealed the presence of putative endogenous DNA mobilization systems (FIG. 16A-FIG. 16C). We wondered whether these native mobilization systems could interface with our engineered pGT vectors and thus performed in vitro conjugations of the MGB strains with laboratory E. coli recipients. Surprisingly, we discovered that MGB4 and MGB9 (both E. fergusonii) were able to mobilize pGT vectors into recipients, although at a lower efficiency than our engineered EcGT2 donor (FIG. 3A, FIG. 16D). These results suggest that some native gut bacteria can promote secondary transfer of engineered payloads using their endogenous conjugation machinery, which may improve payload transfer in situ.

### Recolonization of Transconjugants

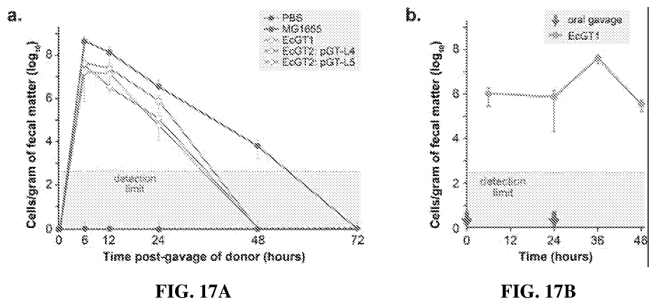

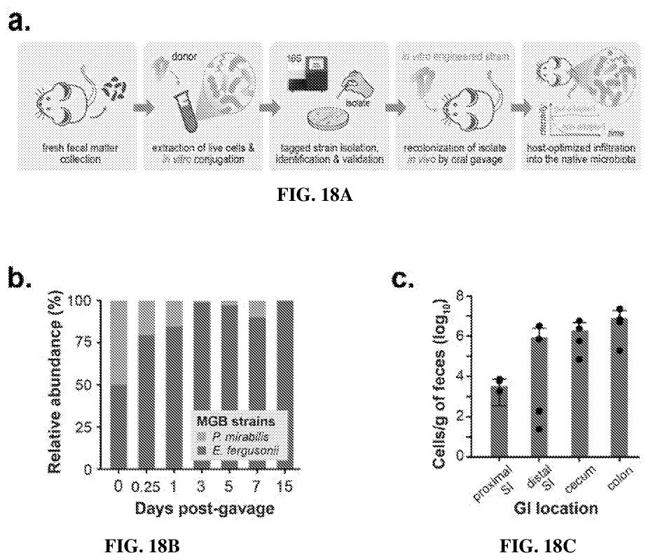

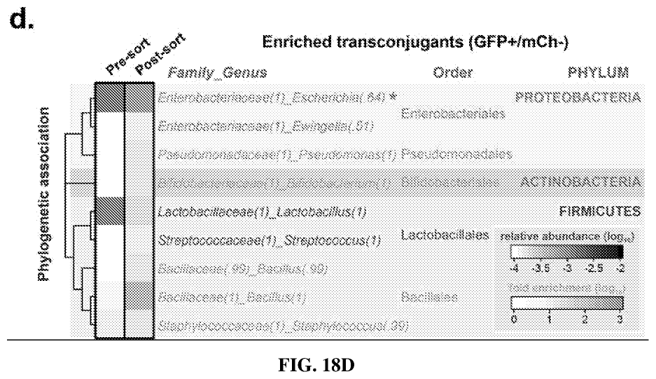

In general, non-gut adapted bacteria (e.g. probiotics) do not colonize an established gut microbiome. Infiltration of foreign species usually requires drastic perturbations, such as use of broad-spectrum antibiotics to suppress the natural flora. Even then, exogenous species do not persist upon discontinuation of antibiotic suppression¹¹. Since our donor strains did not readily colonize the murine gut and transconjugants were lost soon after (FIG. 2B, FIG. 12B, FIG. 17A), we reasoned that using a colonizing donor strain may extend the persistence of payload constructs in situ. To explore this possibility, we tested whether a mixed population of MGB strains (MGB3, MGB4, MGB9) could stably recolonize the native murine gut after a single oral dose without any antibiotic co-administration (FIG. 18A). In contrast to the rapid loss of a non-gut-adapted strain (EcGT1) within 48 hours, MGB strains (especially MGB4) recolonized the murine gut and stably persisted for at least 15 days (FIG. 3B, FIG.18B), populating the entire gastrointestinal tract (FIG. 18C). FACS enrichment and 16S sequencing of GFP-expressing bacteria in feces from these mice revealed transconjugants resulting from in situ transfer of the pGT payload from MGB strains to the native microbiome 6 hours (FIG. 3C) and 11 days post-gavage (FIG. 18D). These transconjugant populations had similar phylogeny although less diversity than those from prior in situ experiments using the non-colonizing EcGT2 donor (FIG. 2C, FIG. 12C). These results highlight the utility of MAGIC to isolate host-derived engineerable strains that can be modified and then used to stably recolonize the native community and mediate further transfer of engineered functions in situ.

In summary, MAGIC enables metagenomic infiltration of genetic payloads into a native microbiome and isolation of genetically modifiable strains from diverse communities. These strains can be reintroduced into their original community to maintain engineered functions via sustained vertical and horizontal transmission in situ. Based on the teachings herein, vector stability and donor strain dosage (FIG. 17B) can be adjusted to better quantitative and temporal control of retention of genetic payloads in situ, which may be useful in applications requiring short-term or long-term actuation of engineered functions¹²⁻¹⁴. Designing genetic programs based on recipient-specific properties enhances targeted execution of desired functions in a defined subset of species in a community^{15, 16}. MAGIC and complementary strategies described herein to engineer the horizontal gene pool can facilitate programmable execution of genetic circuits in other microbial communities¹⁷⁻²⁰. Isolation of genetically tractable representatives from diverse microbiomes will expand the repertoire of new microbial chasses for emerging applications in synthetic biology and microbial ecology.

### Example 7: Methods and Materials Related to Experiments Described in Example 6

Media, chemicals and reagents. E. coli, S. enterica, V cholera, and P. aeruginosa strains were grown in rich LB-Lennox media (BD) buffered to pH 7.45 with NaOH in aerobic conditions at 37° C., while L. reuteri was grown in MRS media (BD). B. thetaiotaomicron and E. faecalis were grown anaerobically at 37° C. in Gifu Anaerobic Modified Medium (GAM) (Nissui Pharmaceutical) or BHI media (BD) supplemented with cysteine (1 g/L), hemin (5 mg/L), resazurin (1 mg/L), and Vitamin K (1 μL/L). All gut bacteria used in the study were grown in LB-Lennox or Gifu Anaerobic Modified Medium (GAM). Antibiotics were used at the following concentrations to select for E. coli: chloramphenicol (chlor) at 20 μg/ml, carbenicillin (carb) at 50 μg/ml, spectinomycin (spec) at 250 μg/ml, kanamycin (kan) at 50 μg/ml, tetracycline (tet) at 25 μg/ml, and erythromycin (erm) at 25 μg/ml. Antibiotics were used at the following ranges of concentrations to select for transconjugant gut bacteria: chloramphenicol (chlor) at 5-20 μg/ml, carbenicillin (carb) at 10-50 μg/ml, tetracycline (tet) at 5-25 μg/ml. Diaminopimelic acid (DAP) was supplemented at 50 μM as needed.

Isolation of live murine gut bacteria. Fresh fecal pellets were harvested from mice, and live gut bacteria were isolated by mechanical homogenization. Briefly, 250 μL of PBS was added to previously weighed pellets in a microcentrifuge tube. Pellets were thoroughly mechanically disrupted using a motorized pellet pestle before adding 750 μL of PBS. The disrupted pellets in PBS were then subjected to four iterations of vortex mixing for 15 sec at medium speed, centrifugation at 1,000 rpm for 30 sec at room temperature, recovery of 750 μL of supernatant into a new tube, and replacement of that volume of PBS before the next iteration. The resulting 3 ml of isolated cells were pelleted by centrifugation at 4,000×g for 5 min at room temperature, the supernatant was discarded, and cells were re-suspended in 0.5-1.0 ml of PBS. All gut bacteria isolations were performed in an anaerobic chamber (Coy Labs).

Donor strain construction. Donor strains EcGT1 and EcGT2 were derived from the S17 λpir E. coli strain²²¹ by generating modifications ΔgalK::mCherry-specR and Δasd::mCherry-specR, respectively, with λ-red recombineering using the pKD46 system²²². Synthetic cassettes containing constitutively active mCherry and spectinomycin resistance genes were constructed with ˜40 bp of homology on both ends to galK or asd flanking regions on the E. coli genome. 100 ng of mCherry-specR cassette DNA were electroporated into recombineering-competent S17-pKD46 cells. Cells were allowed to recover in 3 mL LB+carb at 30° C. for 3 hours prior to plating on LB+spec. Spectinomycin-resistant colonies were genotyped by PCR for validation of mutations. The pKD46 recombineering plasmid was cured out of validated recombinants by growth at 37° C. in the absence of carbenicillin to yield the EcGT1 and EcGT2 strains used throughout the study. When generating the EcGT2 strain, the growth media was supplemented with DAP at all stages of the protocol.

Plasmid construction. pGT vectors were designed to have modular components (e.g., selectable markers, regulatory elements, replication origins) that are interchangeable by isothermal assembly (ITA) or Golden Gate Assembly. Vector selection markers for E. coli were constitutively expressed, while the deliverable cargo or transposase cassettes were expressed using different regulatory elements to enable broad-host or narrow-host range gene expression. Regulatory elements used in this study exhibit a range of activity (Table 1). Vector libraries used in this study are detailed in Table 2. Full vector component sequences are listed in Table 3. The non-transferrable vector pGT-NT used as a negative control was a minimal p15A cloning vector with no origin of transfer, containing a constitutively expressed sfGFP gene.

All plasmids were constructed by isothermal assembly (ITA) with NEBuilder HiFi DNA Assembly Master Mix (New England Biolabs). Component parts were made by high-fidelity PCR with Q5 (NEB) or KAPA Hifi (Kapa Biosystems) polymerases, using existing vectors or gBlocks (Integrated DNA Technologies) as PCR templates. PCR products were digested with DpnI (NEB) and purified with the QIAquick PCR purification kit (Qiagen) prior to ITA and transformation into E. coli. All assembled plasmids were Sanger sequence-verified.

In vitro MAGIC studies on synthetic recipient community. Donor strains harboring pGT vectors and representative recipients (E. coli MG1655, S. enterica ATCC 700931, V. cholera C9503, P. aeruginosa PA01, E. faecalis ATCC 29200, L. reuteri ATCC 23272, B. thetaiotaomicron ATCC 29148) were grown overnight in appropriate media and cultivation conditions, and a 1:1000 dilution culture was re-grown for 14 hours at 37° C. prior to conjugation studies. To prepare cells for in vitro conjugation, donor and recipient populations were washed twice in PBS and cells were quantified by OD₆₀₀ or flow cytometry using SYTO9 staining (Thermo Fisher). 10⁸ donor cells and 10⁸ recipient cells were mixed together, pelleted by centrifugation, and re-suspended in 10 μL PBS. Donor and recipient mixes were spotted on an agar plate and incubated for 5 hours at 30° C. or 37° C. for conjugation. In vitro conjugations were performed on LB-Lennox (E. coli, S. enterica, V. cholera, P. aeruginosa, E. faecalis), MRS (L. reuteri), or supplemented BHI agar (B. thetaiotaomicron). Post-conjugation, cells were scraped from the plate into 1 mL PBS, and 100 uL was plated on appropriate antibiotics and incubated overnight at 30° C. or 37° C. to determine the number of colony forming units (CFU) of transconjugants.

In vitro MAGIC studies on natural recipient community. Donor strains harboring pGT vectors were streaked onto LB-Lennox agar plates with appropriate antibiotics and supplements, grown at 37° C. overnight, and then grown from a single colony in 2 mL liquid media for 10 hours at 37° C. prior to conjugation. The recipient community was isolated anaerobically from fresh murine feces as described above, immediately before conjugation. Donor cells were washed twice in PBS and quantified by OD₆₀₀, while recipient cells were quantified by flow cytometry using SYTO9 staining. 10⁸ donor cells and 10⁹ recipient cells were mixed, pelleted by centrifugation at 5000×g, and resuspended in 25 μL PBS. The mixes were spotted on PBS+1.5% agar plates and incubated at 37° C. either aerobically or anaerobically overnight (9-10 hours). Post-conjugation, cells were scraped from the plate into 1 mL of PBS and subjected to antibiotic selection on GAM media, FACS enrichment, and metagenomic 16S analysis (see below).

In vitro assessment of pGT vectors horizontal gene transfer mediated by natural isolates. MGB natural isolates harboring pGT vectors (MGB3, MGB9, MBG4) were conjugated with a recipient E. coli strain harboring a kanamycin resistance plasmid compatible with pGT vectors. Prior to conjugations, all strains were streaked onto GAM agar plates with appropriate antibiotics, grown at 37° C. overnight, and then grown from a single colony in 5 mL liquid GAM for 10 hours at 37° C. prior to conjugation. MGB donor and recipient cells were washed twice in PBS and quantified by OD₆₀₀. 10⁹ cells each of MGB and recipient strains were mixed, pelleted by centrifugation at 5000×g, and resuspended in 15 μL PBS. The mixtures were spotted on GAM agar plates and incubated at 37° C. aerobically for 6 hours. Post-conjugation, cells were scraped from the plate into 1 mL of PBS and plated on selective and non-selective GAM media. Conjugation efficiency was calculated as

$$\frac{t}{n} ,$$

where t is the number of E. coli transconjugant CFUs and n is the total number of E. coli CFUs.

Measurement of GFP expression in MGB strains. MGB isolates harboring pGT vectors (MGB3, MGB9, MBG4) were streaked onto GAM agar plates with appropriate antibiotics, grown at 37° C. overnight, and then diluted to OD₆₀₀ 0.001 in liquid GAM into a 96 well plate. The plate was incubated in a Synergy H1 (BioTek) microplate reader for 24 hours at 37° C. with orbital shaking. Measurements of OD₆₀₀ and GFP expression (excitation 488 nm, emission 510 nm) were taken using GenS software (BioTek) at the end of 24 hours.

In vivo MAGIC studies in mice. Conventionally raised C57BL/6 female mice (Taconic Biosciences or Charles River Laboratories) were used throughout the study. Two control groups of 4 mice each were gavaged with PBS and EcGT2 containing a non-transferable GFP vector (pGT-NT). Three to four mice were used in each group gavaged with a pGT donor mix or with MGB strains. To equilibrate the murine gut microbiome ahead of time, mice from multiple litters were mixed, co-housed for at least 1 week prior to all experiments, and randomly allocated into groups. Mice were gavaged with 10⁹ donor cells (EcGT2 or MGB strains) in 300 uL of PBS at 8-10 weeks old. Control mice were gavaged with 300 uL of PBS. Fecal matter was collected immediately before gavage and periodically after gavage to analyze the resulting microbiome populations by FACS, metagenomic 16S sequencing, and plating. Upon completion of the study, mice were euthanized and small and large intestinal tissues were extracted. Luminal contents were washed from each tissue sample with PBS and bacteria were extracted by homogenization of the luminal contents for plating and final CFU determination.

Flow cytometry and fluorescence-activated cell sorting (FACS) measurements. Gut bacteria isolated from fresh fecal pellets were analyzed for evidence of successful conjugation on a flow cytometer (Guava easyCyte HT) using red (642 nm) and blue (488 nm) lasers with Red2 and Green photodiodes to detect mCherry (587/610 nm) and sfGFP (485/510 nm) fluorescence, respectively. Bacteria at 100× and 1,000× dilutions in PBS were used for optimal detection of donor (GFP⁺/mCh⁺), gut microbes without a transferred vector (GFP⁻/mCh⁻), and transconjugants (GFP⁺/mCh⁻). Data were collected and analyzed usingInCyte 3.1 software. For FACS enrichment studies, a BD FACS Aria II cell sorter operated with BD FACSDiva software was used to gate for sfGFP (FITC filter 515/10 nm) and mCherry (mCherry filter 616/26 nm). A double gating on GFP and mCherry channels was used to select for cells with GFP⁺/mCh⁻ fluorescence. In addition, background events were also taken into account by using the GFP⁺/mCh⁻ fluorescence detected in the fecal sample prior to gavage as baseline signal. An increase over the baseline signifies an enrichment of transconjugants. Population density (cells/gram fecal matter) was calculated based on number of cells sorted over the mass of the sorted fecal sample. Additional plating and direct colony counting were used to validate flow cytometric measurements. FACS plots were formatted using FCS Express 6.

Fluorescence microscopy of fecal bacteria. Bacteria were suspended in PBS and centrifuged at 5000×g to concentrate into a smaller volume, which varied depending on the concentration of bacteria. The bacteria were resuspended by pipetting, and a volume of 15 uL was dropped onto a Superfrost Plus microscope slide (Thermo Shandon) and covered with a glass cover slip. Slides were air-dried until the PBS receded from the edges of the cover slip and then sealed with clear nail polish. Bacteria were imaged at 40x magnification on a Nikon Eclipse Ti2 microscope on bright field, RFP, and GFP channels using NIS-Elements-AR software.

Validation of pGT vectors in transconjugants. Transconjugant validation was performed by colony PCR of the GFP-antibiotic resistance payload and/or the pGT vector backbone. PCR products with the expected size were further verified by Sanger sequencing. Taxonomy assignment of isolated colonies was based on 16S rRNA PCR amplification and Sanger sequencing. All transconjugant strains validated in the study are listed in Table 4.

In vitro evolution of transconjugant gut bacteria. Escherichia fergusonii transconjugants MGB4 and MGB9 were serially passaged in LB media for 11-15 days. Starting from a single colony, the strains were inoculated into LB and grown at 37 C with shaking. Every 12 hours the liquid culture was diluted 1:1000 into fresh LB media. At selected time points an aliquot of the saturated culture was plated on selective (50 μg/mL carbenicillin) and non-selective plates to quantify the percentage of cells expressing the payload antibiotic resistance and GFP genes. MGB9 cultures were also plated on selective plates with 20 μg/mL chloramphenicol to check for maintenance of the plasmid backbone.

Metagenomic 16S sequencing. Genomic DNA was extracted from isolated bacteria populations using the MasterPure Gram Positive DNA Purification Kit (Epicentre). PCR amplification of the 16S rRNA V4 region and multiplexed barcoding of samples were performed based on previous protocols²³ . The V4 region of the 16S rRNA gene was amplified using customized primers based on the method described in Kozich et al.²²³ with the following modifications: (i) alteration of 16S primers to match updated EMP 505f and 806rB primers²²⁴⁻²⁶ and (ii) use of NexteraXT indices such that each index pair is separated by a Hamming distance of >2 and Illumina low-plex pooling guidelines can be used. Sequencing was performed using the Illumina MiSeq system (500V2 kit).

Analysis of 16S next-generation sequencing (NGS) data. Bacteria from fecal samples taken right before gavage (T0) and 6 hours post-gavage (T6) and were sorted by FACS to enrich for transconjugants. The compositions of the sorted transconjugant and total populations for each sample were determined from 16S sequencing data using the UPARSE pipeline²²⁷ (USEARCH version 10.0.240) to generate Operational Taxonomic Unit (OTU) tables and abundances and the RDP classifier²²⁸ to assign the taxonomy. Phylogenetic associations were analyzed at the genus level with at least 90% confidence for 16S assignment. In all MiSeq runs, two blank controls with sterile water as input material were included to check for contaminants in the reagents and to filter out contaminant OTUs if present. Reads mapping to non-bacterial DNA (e.g., mitochondria, plastids, or other eukaryotic DNA) were also excluded from analysis. Only OTUs with more than 10 reads were considered in downstream analysis.

Relative abundances of OTUs in unsorted total fecal populations were calculated as the normalized number of reads in a sample. Relative abundances of OTUs in TO FACS-enriched populations were used to measure false positive background fluorescence, which was subtracted from the T6 transconjugant populations. The corrected relative abundance of each OTU in a T6 FACS-enriched population is given by the formula:

$$R {A}_{6 , i , sorted} = \frac{{A}_{6 , i} * {N}_{6} - {A}_{0 , i} * {N}_{0}}{{Σ}_{i} ( {A}_{6 , i} * {N}_{6} - {A}_{0 , i} * {N}_{0} )}$$

where RA_{t,i,sorted} is the corrected relative abundance of OTU i at time t, A_{t,i} is the normalized number of reads of OTU i at time t in the FACS-sorted sample, and Nₜ is the fraction of mCherry-/GFP+ FACS-sorted events at time t. OTUs for which RA_{6,i,sorted} is negative are eliminated from subsequent analysis, and all remaining RA_{6,i,sorted} values are renormalized.

The fold enrichment of each OTU in the FACS-sorted population is defined as its relative abundance in the FACS-sorted population divided by its relative abundance in the unsorted total population at T6. To overcome the problem of detection limits (i.e., OTU i appears in the sorted population but is below the detection limit in the total population), we added a pseudo-count of p to all relative abundances when calculating fold enrichments. p is given by

p=10^{└-log}^{₁₀} ^{n┘}

where n is the total number of reads in the FACS-sorted sample and └−log₁₀ n┘ is the floor function—the greatest integer less than or equal to—of −log₁₀ n. The fold enrichment of OTU i with the pseudo-count correction is calculated as

$${F}_{i} = \frac{R {A}_{6 , i , sorted} + p}{R {A}_{6 , i , unsorted} + p}$$

If the relative abundance of OTU i in the unsorted population is below the detection limit, then the fold enrichment is calculable as

$$\frac{R {A}_{6 , i , sorted} + p}{p} ,$$

instead of

$$\frac{R {A}_{6 , i , sorted}}{0} .$$

The pseudo-count-corrected fold enrichment Fᵢ overestimates the true fold enrichment

$$( \frac{R {A}_{6 , i , sorted}}{R {A}_{6 , i , unsorted}} )$$

by at most 10%, while possibly underestimating it. Because

$$0 < p \leq \frac{1}{n} and {RA}_{6 , i , sorted} \geq \frac{10}{n} ,$$

$${F}_{i} = \frac{R {A}_{6 , i , sorted} + p}{R {A}_{6 , i , unsorted} + p} \leq \frac{R {A}_{6 , i , sorted} + p}{R {A}_{6 , i , unsorted}} \leq \frac{1.1 * R {A}_{6 , i , sorted}}{R {A}_{6 , i , unsorted}}$$

In all heat maps showing fold enrichment versus relative abundance, only OTUs with Fᵢ>10 are displayed to show more stringent and high confidence results. R code for this analysis is available upon request.

Whole genome sequencing of engineered mouse gut bacteria (MGB) isolates. To sequence MGB isolates, we prepared a sequencing library using the Nextera kit (Illumina) and utilized the Illumina HiSeq 2500 platform for 100 bp single-end reads. The SPAdes single cell assembler pipeline (version 3.9.1)²²⁹ was employed to generate whole genome contigs. BLAST and PlasmidFinder (version 1.3)³⁰ were used to analyze the sequences and identify native mobilization systems. Geneious (version 7.1.5) was used to visualize contig alignments to genomes and plasmids.

### REFERENCES FOR EXAMPLES 6 AND 7

1. Lagier, J. C. et al. Culture of previously uncultured members of the human gut microbiota by culturomics. Nat Microbiol 1, 16203 (2016).

2. Yaung, S. J., Church, G. M. & Wang, H. H. Recent progress in engineering human-associated microbiomes. Methods in molecular biology 1151, 3-25 (2014).

3. Cuiv, P. O. et al. Isolation of Genetically Tractable Most-Wanted Bacteria by Metaparental Mating. Sci Rep 5, 13282 (2015).

4. Mimee, M., Tucker, A. C., Voigt, C. A. & Lu, T. K. Programming a Human Commensal Bacterium, Bacteroides thetaiotaomicron, to Sense and Respond to Stimuli in the Murine Gut Microbiota. Cell Syst 1, 62-71 (2015).

5. Tock, M. R. & Dryden, D. T. The biology of restriction and anti-restriction. Curr Opin Microbiol 8, 466-472 (2005).

6. Marraffini, L. A. CRISPR-Cas immunity in prokaryotes. Nature 526, 55-61 (2015).

7. Thomas, C. M. & Nielsen, K. M. Mechanisms of, and barriers to, horizontal gene transfer between bacteria. Nat Rev Microbiol 3, 711-721 (2005).

8. Human Microbiome Project, C. Structure, function and diversity of the healthy human microbiome. Nature 486, 207-214 (2012).

9. Pansegrau, W. et al. Complete nucleotide sequence of Birmingham IncP alpha plasmids. Compilation and comparative analysis. J Mol Biol 239, 623-663 (1994).

10. Hapfelmeier, S. et al. Reversible microbial colonization of germ-free mice reveals the dynamics of IgA immune responses. Science 328, 1705-1709 (2010).

11. Myhal, M. L., Laux, D. C. & Cohen, P. S. Relative colonizing abilities of human fecal and K 12 strains of Escherichia coli in the large intestines of streptomycin-treated mice. Eur J Clin Microbiol 1, 186-192 (1982).

12. Kommineni, S. et al. Bacteriocin production augments niche competition by enterococci in the mammalian gastrointestinal tract. Nature 526, 719-722 (2015).

13. Saeidi, N. et al. Engineering microbes to sense and eradicate Pseudomonas aeruginosa, a human pathogen. Mol Syst Biol 7, 521 (2011).

14. Steidler, L. et al. Treatment of murine colitis by Lactococcus lactis secreting interleukin-10. Science 289, 1352-1355 (2000).

15. Wegmann, U., Horn, N. & Carding, S. R. Defining the bacteroides ribosomal binding site. Applied and environmental microbiology 79, 1980-1989 (2013).

16. Sheth, R. U., Cabral, V., Chen, S. P. & Wang, H. H. Manipulating Bacterial Communities by in situ Microbiome Engineering. Trends Genet 32, 189-200 (2016).

17. Klumper, U. et al. Broad host range plasmids can invade an unexpectedly diverse fraction of a soil bacterial community. The ISME journal 9, 934-945 (2015).

18. Dahlberg, C., Bergstrom, M. & Hermansson, M. In Situ Detection of High Levels of Horizontal Plasmid Transfer in Marine Bacterial Communities. Appl Environ Microbiol 64, 2670-2675 (1998).

19. Bikard, D. et al. Exploiting CRISPR-Cas nucleases to produce sequence-specific antimicrobials. Nature biotechnology 32, 1146-1150 (2014).

20. Brophy, J. A. N. et al. Engineered integrative and conjugative elements for efficient and inducible DNA transfer to undomesticated bacteria. Nat Microbiol 3, 1043-1053 (2018).

21. Simon, R., Priefer, U. & Puhler, A. A Broad Host Range Mobilization System for In Vivo Genetic Engineering: Transposon Mutagenesis in Gram Negative Bacteria. Nat Biotech 1, 784-791 (1983).

22. Datsenko, K. A. & Wanner, B. L. One-step inactivation of chromosomal genes in Escherichia coli K-12 using PCR products. P Natl Acad Sci USA 97, 6640-6645 (2000).

23. Kozich, J. J., Westcott, S. L., Baxter, N. T., Highlander, S. K. & Schloss, P. D. Development of a dual-index sequencing strategy and curation pipeline for analyzing amplicon sequence data on the MiSeq Illumina sequencing platform. Applied and environmental microbiology 79, 5112-5120 (2013).

24. Caporaso, J. G. et al. Global patterns of 16S rRNA diversity at a depth of millions of sequences per sample. Proceedings of the National Academy of Sciences of the United States of America 108 Suppl 1, 4516-4522 (2011).

25. Parada, A. E., Needham, D. M. & Fuhrman, J. A. Every base matters: assessing small subunit rRNA primers for marine microbiomes with mock communities, time series and global field samples. Environ Microbiol 18, 1403-1414 (2016).

26. Apprill, A., McNally, S., Parsons, R. & Weber, L. Minor revision to V4 region SSU rRNA 806R gene primer greatly increases detection of SAR11 bacterioplankton. Aquatic Microbial Ecology 75, 129-137 (2015).

27. Edgar, R. C. UPARSE: highly accurate OTU sequences from microbial amplicon reads. Nat Methods 10, 996-998 (2013).

28. Wang, Q., Garrity, G. M., Tiedje, J. M. & Cole, J. R. Naive Bayesian classifier for rapid assignment of rRNA sequences into the new bacterial taxonomy. Appl Environ Microb 73, 5261-5267 (2007).

29. Nurk, S. et al. Assembling single-cell genomes and mini-metagenomes from chimeric MDA products. J Comput Biol 20, 714-737 (2013).

30. Carattoli, A. et al. In silico detection and typing of plasmids using PlasmidFinder and plasmid multilocus sequence typing. Antimicrob Agents Chemother 58, 3895-3903 (2014).

31. Stalker, D. M., Kolter, R. & Helinski, D. R. Nucleotide sequence of the region of an origin of replication of the antibiotic resistance plasmid R6K. Proceedings of the National Academy of Sciences of the United States of America 76, 1150-1154 (1979).

32. Hiszczynska-Sawicka, E. & Kur, J. Effect of Escherichia coli IHF mutations on plasmid p15A copy number. Plasmid 38, 174-179 (1997).

33. Kues, U. & Stahl, U. Replication of plasmids in gram-negative bacteria. Microbiol Rev 53, 491-516 (1989).

34. Antoine, R. & Locht, C. Isolation and molecular characterization of a novel broad-host-range plasmid from Bordetella bronchiseptica with sequence similarities to plasmids from gram-positive organisms. Mol Microbiol 6, 1785-1799 (1992).

35. Frey, J., Bagdasarian, M. M. & Bagdasarian, M. Replication and copy number control of the broad-host-range plasmid RSF1010. Gene 113, 101-106 (1992).

36. Bryksin, A. V. & Matsumura, I. Rational design of a plasmid origin that replicates efficiently in both gram-positive and gram-negative bacteria. PloS one 5, e13244 (2010).

37. Lampe, D. J., Akerley, B. J., Rubin, E. J., Mekalanos, J. J. & Robertson, H. M. Hyperactive transposase mutants of the Himar1 mariner transposon. Proc Natl Acad Sci US A 96, 11428-11433 (1999).

TABLE 1
List of vectors and vector components.
Origins of replication (oriR):

| Origin | Copy # | Host range | Code |
|---|---|---|---|
| R6K¹ | 10-20 | Narrow (Proteobacteria) | K |
| p15A² | 14-16 | Narrow (Enterobacteria) | A |
| oriV³ | 4-7 | Broad (Gram- and Gram+) | V |
| pBBR1⁴ | 15-40 | Broad (preferably Gram-) | B |
| RSF1010⁵ | 12 | Broad (Gram- and Gram+) | S |
| RCR⁶ | 250-350 | Broad (Eubacteria) | W |

Integrative elements:

| Transposase | Transposon inverted repeat sequence | Host range | Code |
|---|---|---|---|
| none | — | — | — |
| Himar⁷ (SEQ | ACAGGTTGGATGATAAGTCCCCGGTCT | Broad | h |
| ID NO. 9) | | | |
| Tn5 (SEQ ID | CTGTCTCTTATACACATCT | Broad | t |
| NO. 10) | | | |

Regulation sequences (Code Column also represents the SEQ ID NO.):

| | Expression | | |
|---|---|---|---|
| Promoter UTR | in E. coli | Origin of sequence | Code |
| GATTGCATTAGGTTTTAGTTTCTTGTATAATGC | +++ | Bacillus cellulosilyticus | 1 |
| TTAATGTTGGTCACTGACAGGCTACGATACGG | | | |
| AAGGTTGCTCACGCCCGGCCCCTTTGCCATGGCT | | | |
| AGTGTGTGGAAATTTCCGAGGAGCAAGTCTAT | | | |
| TTCCAAAAATGGGCGAAAAAGGAGGTAATAC | | | |
| A | | | |
| GGGAGAGCTTCAACGGCGCTTCTACCCATTTGC | + | Geobacillus sp. | 2 |
| TTGGAAAGGATGAGGAGCAGGAAGAAATTCCG | | | |
| TCCCCAATGCGACGGCCCTTTACATCCATGTTG | | | |
| TTTGATAGTATAATGGATACGGATTGACCAAA | | | |
| TTGTTCATTTAGTCAGTTTGAAGGATGAGGAGT | | | |
| GTGAAGGATACGGCTGCGGCACTTCGACATCGC | | Eggerthella lenta | 3 |
| CCCATGTGGCGGCTTTGAACTGGGCTTATGAAA | | | |
| CGCGTTCACAACCTTTTTTGACCATCGGCGCGA | | | |
| ACGTGGTATCATGCGTTCAGCTTTTGCCCATAC | | | |
| ATACTACGTGCTCAATCTAGGAGGATTTCATA | | | |
| C | | | |
| CTCTAGAGTAGTAGATTATTTTAGGAATTTAG | + | Segmented filamentous | 4 |
| ATGTTTTGTATGAAATAGATGCTTCGTATGGA | | bacteria | |
| ATTAATGAAATTTTTAGTCAGGTAAAAAAGG | | | |
| TAATAGGAGAATATT | | | |
| CTTTTAAATGATGAAAAGAAATATTTAGGGA | ++ | Segmented filamentous | 5 |
| AGATTGTTTCGACGCGAATTTTTGATCTTGAA | | bacteria | |
| AATGATCACCTTATCGGACAAGCTTTAAAAT | | | |
| AGGAGGATATAAAAAT | | | |
| ATAAGGATTCTTTAAAGAGAGATATAGTTAT | + | Segmented filamentous | 6 |
| CTCAAAGACTGTAGAATTTTTACTAAATCAA | | bacteria | |
| AATAAAAAAAGAGGTATTAAATAGAGTGTA | | | |
| TTTTAAAGGAGGAGACTT | | | |
| AAACACCAATAAAATTAGAATATTTAGGAGC | + | Segmented filamentous | 7 |
| GACTTTAAAAAAGTTTAATAAGAATTGTTTA | | bacteria | |
| TGAGATATTTTTATTATATTTAAACTCAATTT | | | |
| AAAGTAGGGAGAATAG | | | |
| GCAACTGTTCAAGAAGTTATTAACTCGGGAGT | + | Clostridium perfringens | 8 |
| CCAGTCGAAGTGGGCAAGTTGAAAAATTCACA | | | |
| AAAATGTGGTATAATATCTTTGTTCATTAGAG | | | |
| CGATAAACTTGAATTTGAGACGGAACTTAG | | | |

Vector selection genes:

| Resistance gene | Antibiotic selection | [Ab] in E. coli |
|---|---|---|
| Beta-lactamase | Carbenicillin | 50 μg/ml |
| Chlor | Chloramphenicol | 20 μg/ml |
| Tet | Tet | 25 μg/ml |
| Spec | Spec | 250 μg/ml |
| Kan | Kan | 50 μg/ml |

Cargo selection cassettes:

| Resistance cassette | Antibiotic selection | | [Ab] in E. coli | |
|---|---|---|---|---|
| GFP-Beta-lactamase | Carb | | 50 μg/ml | |
| GFP-CatP | Chlor | | 20 μg/ml | |
| GFP-Tet | Tet | | 25 μg/ml | |
| GFP-Spec | Spec | | 250 μg/ml | |
| GFP-Kan | Kan | | 50 μg/ml | |
| GFP-ErmG | Erm | | — | |
| | Cargo | Cargo | Vector | Transposase |
| Vector name | selection | promoter | selection | promoter |
| pGT-Ah1 | GFP-Beta- | 4 | Chlor | 4 |
| | lactamase | | | |
| pGT-Ah2 | GFP-Beta- | 5 | Chlor | 5 |
| | lactamase | | | |
| pGT-Ah3 | GFP-Beta- | 6 | Chlor | 6 |
| | lactamase | | | |
| pGT-Ah4 | GFP-Beta- | 7 | Chlor | 7 |
| | lactamase | | | |
| pGT-Ah5 | GFP-CatP | 8 | Kan | 4 |
| pGT-Ah6 | GFP-CatP | 8 | Kan | 5 |
| pGT-Ah7 | GFP-CatP | 8 | Kan | 6 |
| pGT-Ah8 | GFP-CatP | 8 | Kan | 7 |
| pGT-Ah9 | GFP-Tet | 4 | Chlor | 4 |
| pGT-Ah10 | GFP-Tet | 4 | Chlor | 5 |
| pGT-Ah11 | GFP-Tet | 4 | Chlor | 6 |
| pGT-B1 | GFP | 1 | Beta-lactamase | — |
| pGT-B2 | GFP | 2 | Beta-lactamase | — |
| pGT-B3 | GFP | 3 | Beta-lactamase | — |
| pGT-S1 | GFP-Beta- | 4 | Beta-lactamase | — |
| | lactamase | | | |
| pGT-S2 | GFP-Beta- | 5 | Beta-lactamase | — |
| | lactamase | | | |
| pGT-S3 | GFP-Tet | 4 | Tet | — |
| pGT-S4 | GFP-Tet | 5 | Tet | — |
| pGT-Kh1 | GFP-Beta- | 4 | Chlor | 4 |
| | lactamase | | | |
| pGT-Kh2 | GFP-Beta- | 5 | Chlor | 5 |
| | lactamase | | | |
| pGT-Kh3 | GFP-Beta- | 7 | Chlor | 7 |
| | lactamase | | | |

TABLE 2
Vector libraries used in this study.

| Library | Vectors |
|---|---|
| pGT-Ll | B1, B2, B3 |
| pGT-L2 | Ah5, Ah6, Ah7, Ah8 |
| pGT-L3 | S1, S2, S3, S4 |
| pGT-L4 | Ahl, Ah3, Bl,B2, B3 |
| pGT-L5 | Ah5, Ah6, Ah7, Ah8, Ah9, AhlO, Ahl 1 |
| pGT-L6 | Ahl, Ah3, Ah5, Ah6, Ah7, Ah8, Ah9, AhlO, Ahll, B1, B2, B3 |
| pGT-L7 | Ahl, Ah2, Ah3, Ah4 |
| pGT-L8 | Khl, Kh2, Kh3 |

TABLE 3
Full sequences of pGT vector parts
Cargo selection genes
Beta-lactamase (carbenicillin/ampicillin resistance)-SEQ ID NO: 13
ATGAGTATTCAACATTTCCGTGTCGCCCTTATTCCCTTTTTTGCGGCATTTTGCCTTCCTGTT
TTTGCTCACCCAGAAACGCTGGTGAAAGTAAAAGATGCTGAAGATCAGTTGGGTGCACGAGTGGG
TTACATCGAACTGGATCTCAACAGCGGTAAGATCCTTGAGAGTTTTCGCCCCGAAGAACGTTTTCC
AATGATGAGCACTTTTAAAGTTCTGCTATGTGGCGCGGTATTATCCCGTATTGACGCCGGGCAAGA
GCAACTCGGTCGCCGCATACACTATTCTCAGAATGACTTGGTTGAGTACTCACCAGTCACAGAAAA
GCATCTTACGGATGGCATGACAGTAAGAGAATTATGCAGTGCTGCCATAACCATGAGTGATAACA
CTGCGGCCAACTTACTTCTGACAACGATCGGAGGACCGAAGGAGCTAACCGCTTTTTTGCACAACA
TGGGGGATCATGTAACTCGCCTTGATCGTTGGGAACCGGAGCTGAATGAAGCCATACCAAACGAC
GAGCGTGACACCACGATGCCTGTAGCAATGGCAACAACGTTGCGCAAACTATTAACTGGCGAACT
ACTTACTCTAGCTTCCCGGCAACAATTAATAGACTGGATGGAGGCGGATAAAGTTGCAGGACCAC
TTCTGCGCTCGGCCCTTCCGGCTGGCTGGTTTATTGCTGATAAATCTGGAGCCGGTGAGCGTGGGT
CTCGCGGTATCATTGCAGCACTGGGGCCAGATGGTAAGCCCTCCCGTATCGTAGTTATCTACACGA
CGGGGAGTCAGGCAACTATGGATGAACGAAATAGACAGATCGCTGAGATAGGTGCCTCACTGATT
AAGCATTGGTAA
CatP (chloramphenicol resistance)-SEQ ID NO: 14
ATGGTATTTGAAAAAATTGATAAAAATAGTTGGAACAGAAAAGAGTATTTTGACCACTAC
TTTGCAAGTGTACCTTGTACATACAGCATGACCGTTAAAGTGGATATCACACAAATAAAGGAAAA
GGGAATGAAACTATATCCTGCAATGCTTTATTATATTGCAATGATTGTAAACCGCCATTCAGAGTT
TAGGACGGCAATCAATCAAGATGGTGAATTGGGGATATATGATGAGATGATACCAAGCTATACAA
TATTTCACAATGATACTGAAACATTTTCCAGCCTTTGGACTGAGTGTAAGTCTGACTTTAAATCATT
TTTAGCAGATTATGAAAGTGATACGCAACGGTATGGAAACAATCATAGAATGGAAGGAAAGCCAA
ATGCTCCGGAAAACATTTTTAATGTATCTATGATACCGTGGTCAACCTTCGATGGCTTTAATCTGAA
TTTGCAGAAAGGATATGATTATTTGATTCCTATTTTTACTATGGGGAAATATTATAAAGAAGATAA
CAAAATTATACTTCCTTTGGCAATTCAAGTTCATCACGCAGTATGTGACGGATTTCACATTTGCCGT
TTTGTAAACGAATTGCAGGAATTGATAAATAGTTAA Derived from pJIR750 plasmid from Clostridium
perfringens
Tet (tetracycline resistance)-SEQ ID NO: 15
ATGAATATTATAAATTTAGGAATTCTTGCTCACATTGATGCAGGAAAAACTTCCGTAACCG
AGAATCTGCTGTTTGCCAGTGGAGCAACGGAAAAGTGCGGCTGTGTGGATAATGGTGACACCATA
ACGGACTCTATGGATATAGAGAAACGTAGAGGAATTACTGTTCGGGCTTCTACGACATCTATTATC
TGGAATGGTGTGAAATGCAATATCATTGACACTCCGGGACACATGGATTTTATTGCGGAAGTGGA
GCGGACATTCAAAATGCTTGATGGAGCAGTCCTCATCTTATCCGCAAAGGAAGGCATACAAGCGC
AGACAAAGTTGCTGTTCAATACTTTACAGAAGCTGCAAATCCCGACAATTATATTTATCAATAAGA
TTGACCGAGCCGGTGTGAATTTGGAGCGTTTGTATCTGGATATAAAAGCAAATCTGTCTCAAGATG
TCCTGTTTATGCAAAATGTTGTCGATGGATCGGTTTATCCGGTTTGCTCCCAAACATATATAAAGG
AAGAATACAAAGAATTTGTATGCAACCATGACGACAATATATTAGAACGATATTTGGCGGATAGC
GAAATTTCACCGGCTGATTATTGGAATACGATAATCGCTCTTGTGGCAAAAGCCAAAGTCTATCCG
GTGCTACATGGATCAGCAATGTTCAATATCGGTATCAATGAGTTGTTGGACGCCATCACTTCTTTTA
TACTTCCTCCGGCATCGGTCTCAAACAGACTTTCATCTTATCTTTATAAGATAGAGCATGACCCCAA
AGGACATAAAAGAAGTTTTCTAAAAATAATTGACGGAAGTCTGAGACTTCGAGACGTTGTAAGAA
TCAACGATTCGGAAAAATTCATCAAGATTAAAAATCTAAAAACTATCAATCAGGGCAGAGAGATA
AATGTTGATGAAGTGGGCGCCAATGATATCGCGATTGTAGAGGATATGGATGATTTTCGAATCGG
AAATTATTTAGGTGCTGAACCTTGTTTGATTCAAGGATTATCGCATCAGCATCCCGCTCTCAAATCC
TCCGTCCGGCCAGACAGGCCCGAAGAGAGAAGCAAGGTGATATCCGCTCTGAATACATTGTGGAT
TGAAGACCCGTCTTTGTCCTTTTCCATAAACTCATATAGTGATGAATTGGAAATCTCGTTATATGGT
TTAACCCAAAAGGAAATCATACAGACATTGCTGGAAGAACGATTTTCCGTAAAGGTCCATTTTGAT
GAGATCAAGACTATATACAAAGAACGACCTGTAAAAAAGGTCAATAAGATTATTCAGATCGAAGT
GCCGCCCAACCCTTATTGGGCCACAATAGGGCTGACTCTTGAACCCTTACCGTTAGGGACAGGGTT
GCAAATCGAAAGTGACATCTCCTATGGTTATCTGAACCATTCTTTTCAAAATGCCGTTTTTGAAGG
GATTCGTATGTCTTGCCAATCCGGGTTACATGGATGGGAAGTGACTGATCTGAAAGTAACTTTTAC
TCAAGCCGAGTATTATAGCCCGGTAAGTACACCTGCTGATTTCAGACAGCTGACCCCTTATGTCTT
CAGGCTGGCCTTGCAACAGTCAGGTGTGGACATTCTCGAACCGATGCTCTATTTTGAGTTGCAGAT
ACCCCAAGCGGCAAGTTCCAAAGCTATTACAGATTTGCAAAAAATGATGTCTGAGATTGAAGACA
TCAGTTGCAATAATGAGTGGTGTCATATTAAAGGGAAAGTTCCATTAAATACAAGTAAAGACTAT
GCATCAGAAGTAAGTTCATACACTAAGGGCTTAGGCATTTTTATGGTTAAGCCATGCGGGTATCAA
ATAACAAAAGGCGGTTATTCTGATAATATCCGCATGAACGAAAAAGATAAACTTTTATTCATGTTC
CAAAAATCAATGTCATCAAAATAA
Spec (spectinomycin resistance)-SEQ ID NO: 16
ATGCGCTCACGCAACTGGTCCAGAACCTTGACCGAACGCAGCGGTGGTAACGGCGCAGTG
GCGGTTTTCATGGCTTGTTATGACTGTTTTTTTGGGGTACAGTCTATGCCTCGGGCATCCAAGCAGC
AAGCGCGTTACGCCGTGGGTCGATGTTTGATGTTATGGAGCAGCAACGATGTTACGCAGCAGGGC
AGTCGCCCTAAAACAAAGTTAAACATCATGAGGGAAGCGGTGATCGCCGAAGTATCGACTCAACT
ATCAGAGGTAGTTGGCGTCATCGAGCGCCATCTCGAACCGACGTTGCTGGCCGTACATTTGTACGG
CTCCGCAGTGGATGGCGGCCTGAAGCCACACAGTGATATTGATTTGCTGGTTACGGTGACCGTAAG
GCTTGATGAAACAACGCGGCGAGCTTTGATCAACGACCTTTTGGAAACTTCGGCTTCCCCTGGAGA
GAGCGAGATTCTCCGCGCTGTAGAAGTCACCATTGTTGTGCACGACGACATCATTCCGTGGCGTTA
TCCAGCTAAGCGCGAACTGCAATTTGGAGAATGGCAGCGCAATGACATTCTTGCAGGTATCTTCGA
GCCAGCCACGATCGACATTGATCTGGCTATCTTGCTGACAAAAGCAAGAGAACATAGCGTTGCCTT
GGTAGGTCCAGCGGCGGAGGAACTCTTTGATCCGGTTCCTGAACAGGATCTATTTGAGGCGCTAAA
TGAAACCTTAACGCTATGGAACTCGCCGCCCGACTGGGCTGGCGATGAGCGAAATGTAGTGCTTA
CGTTGTCCCGCATTTGGTACAGCGCAGTAACCGGCAAAATCGCGCCGAAGGATGTCGCTGCCGACT
GGGCAATGGAGCGCCTGCCGGCCCAGTATCAGCCCGTCATACTTGAAGCTAGACAGGCTTATCTTG
GACAAGAAGAAGATCGCTTGGCCTCGCGCGCAGATCAGTTGGAAGAATTTGTCCACTACGTGAAA
GGCGAGATCACCAAGGTAGTCGGCAAATAA
Kan (kanamycin resistance)-SEQ ID NO: 17
ATGAGCCATATTCAACGGGAAACGTCGAGGCCGCGATTAAATTCCAACATGGATGCTGAT
TTATATGGGTATAAATGGGCTCGCGATAATGTCGGGCAATCAGGTGCGACAATCTATCGCTTGTAT
GGGAAGCCCGATGCGCCAGAGTTGTTTCTGAAACATGGCAAAGGTAGCGTTGCCAATGATGTTAC
AGATGAGATGGTCAGACTAAACTGGCTGACGGAATTTATGCCTCTTCCGACCATCAAGCATTTTAT
CCGTACTCCTGATGATGCATGGTTACTCACCACTGCGATCCCCGGAAAAACAGCATTCCAGGTATT
AGAAGAATATCCTGATTCAGGTGAAAATATTGTTGATGCGCTGGCAGTGTTCCTGCGCCGGTTGCA
TTCGATTCCTGTTTGTAATTGTCCTTTTAACAGCGATCGCGTATTTCGTCTCGCTCAGGCGCAATCA
CGAATGAATAACGGTTTGGTTGATGCGAGTGATTTTGATGACGAGCGTAATGGCTGGCCTGTTGAA
CAAGTCTGGAAAGAAATGCATAAACTTTTGCCATTCTCACCGGATTCAGTCGTCACTCATGGTGAT
TTCTCACTTGATAACCTTATTTTTGACGAGGGGAAATTAATAGGTTGTATTGATGTTGGACGAGTC
GGAATCGCAGACCGATACCAGGATCTTGCCATCCTATGGAACTGCCTCGGTGAGTTTTCTCCTTCA
TTACAGAAACGGCTTTTTCAAAAATATGGTATTGATAATCCTGATATGAATAAATTGCAGTTTCAT
TTGATGCTCGATGAGTTTTTCTAA
ermG (erythromycin resistance)-SEQ ID NO: 18
ATGAACAAAGTAAATATAAAAGATAGTCAAAATTTTATTACTTCAAAATATCACATAGAA
AAAATAATGAATTGCATAAGTTTAGATGAAAAAGATAACATCTTTGAAATAGGTGCAGGGAAAGG
TCATTTTACTGCTGGATTGGTAAAGAGATGTAATTTTGTAACGGCGATAGAAATTGATTCTAAATT
ATGTGAGGTAACTCGTAATAAGCTCTTAAATTATCCTAACTATCAAATAGTAAATGATGATATACT
GAAATTTACATTTCCTAGCCACAATCCATATAAAATATTTGGCAGCATACCTTACAACATAAGCAC
AAATATAATTCGAAAAATTGTTTTTGAAAGTTCAGCCACAATAAGTTATTTAATAGTGGAATATGG
TTTTGCTAAAATGTTATTAGATACAAACAGATCACTAGCATTGCTGTTAATGGCAGAGGTAGATAT
TTCTATATTAGCAAAAATTCCTAGGTATTATTTCCATCCAAAACCTAAAGTGGATAGCACATTAAT
TGTATTAAAAAGAAAGCCAGCAAAAATGGCATTTAAAGAGAGAAAAAAATATGAAACTTTTGTAA
TGAAATGGGTTAACAAAGAGTACGAAAAACTGTTTACAAAAAATCAATTTAATAAAGCTTTAAAA
CATGCGAGAATATATGATATAAACAATATTAGTTTCGAACAATTTGTATCGCTATTTAATAGTTAT
AAAATATTTAACGGCTAA
sfGFP-SEQ ID NO: 19
ATGCGTAAAGGCGAAGAGCTGTTCACTGGTTTCGTCACTATTCTGGTGGAACTGGATGGTG
ATGTCAACGGTCATAAGTTTTCCGTGCGTGGCGAGGGTGAAGGTGACGCAACTAATGGTAAACTG
ACGCTGAAGTTCATCTGTACTACTGGTAAACTGCCGGTACCTTGGCCGACTCTGGTAACGACGCTG
ACTTATGGTGTTCAGTGCTTTGCTCGTTATCCGGACCACATGAAGCAGCATGACTTCTTCAAGTCCG
CCATGCCGGAAGGCTATGTGCAGGAACGCACGATTTCCTTTAAGGATGACGGCACGTACAAAACG
CGTGCGGAAGTGAAATTTGAAGGCGATACCCTGGTAAACCGCATTGAGCTGAAAGGCATTGACTT
TAAAGAAGACGGCAATATCCTGGGCCATAAGCTGGAATACAATTTTAACAGCCACAATGTTTACA
TCACCGCCGATAAACAAAAAAATGGCATTAAAGCGAATTTTAAAATTCGCCACAACGTGGAGGAT
GGCAGCGTGCAGCTGGCTGATCACTACCAGCAAAACACTCCAATCGGTGATGGTCCTGTTCTGCTG
CCAGACAATCACTATCTGAGCACGCAAAGCGTTCTGTCTAAAGATCCGAACGAGAAACGCGATCA
CATGGTTCTGCTGGAGTTCGTAACCGCAGCGGGCATCACGCATGGTATGGATGAACTGTACAAATA
A
Vector selection genes
Chlor (chloramphenicol resistance)-SEQ ID NO: 20
ATGGAGAAAAAAATCACTGGATATACCACCGTTGATATATCCCAATGGCATCGTAAAGAA
CATTTTGAGGCATTTCAGTCAGTTGCTCAATGTACCTATAACCAGACCGTTCAGCTGGATATTACG
GCCTTTTTAAAGACCGTAAAGAAAAATAAGCACAAGTTTTATCCGGCCTTTATTCACATTCTTGCC
CGCCTGATGAATGCTCATCCGGAATTACGTATGGCAATGAAAGACGGTGAGCTGGTGATATGGGA
TAGTGTTCACCCTTGTTACACCGTTTTCCATGAGCAAACTGAAACGTTTTCATCGCTCTGGAGTGAA
TACCACGACGATTTCCGGCAGTTTCTACACATATATTCGCAAGATGTGGCGTGTTACGGTGAAAAC
CTGGCCTATTTCCCTAAAGGGTTTATTGAGAATATGTTTTTCGTCTCAGCCAATCCCTGGGTGAGTT
TCACCAGTTTTGATTTAAACGTGGCCAATATGGACAACTTCTTCGCCCCCGTTTTCACCATGGGCAA
ATATTATACGCAAGGCGACAAGGTGCTGATGCCGCTGGCGATTCAGGTTCATCATGCCGTTTGTGA
TGGCTTCCATGTCGGCAGAATGCTTAATGAATTACAACAGTACTGCGATGAGTGGCAGGGCGGGG
CGTAA All vector selection markers not listed here are the same as the ones in the
″Cargo selection genes″ section.
Origins of replication/Plasmid backbones
R6K origin of replication-SEQ ID NO: 21
ATCCCTGGCTTGTTGTCCACAACCGTTAAACCTTAAAAGCTTTAAAAGCCTTATATATTCTT
TTTTTTCTTATAAAACTTAAAACCTTAGAGGCTATTTAAGTTGCTGATTTATATTAATTTTATTGTTC
AAACATGAGAGCTTAGTACGTGAAACATGAGAGCTTAGTACGTTAGCCATGAGAGCTTAGTACGT
TAGCCATGAGGGTTTAGTTCGTTAAACATGAGAGCTTAGTACGTTAAACATGAGAGCTTAGTACGT
GAAACATGAGAGCTTAGTACGTACTATCAACAGGTTGAACTGCTGATCTTC Requires additional
pir gene for replication
p15A origin of replication-SEQ ID NO: 22
AACAACTTATATCGTATGGGGCTGACTTCAGGTGCTACATTTGAAGAGATAAATTGCACTG
AAATCTAGAAATATTTTATCTGATTAATAAGATGATCTTCTTGAGATCGTTTTGGTCTGCGCGTAAT
CTCTTGCTCTGAAAACGAAAAAACCGCCTTGCAGGGCGGTTTTTCGAAGGTTCTCTGAGCTACCAA
CTCTTTGAACCGAGGTAACTGGCTTGGAGGAGCGCAGTCACCAAAACTTGTCCTTTCAGTTTAGCC
TTAACCGGCGCATGACTTCAAGACTAACTCCTCTAAATCAATTACCAGTGGCTGCTGCCAGTGGTG
CTTTTGCATGTCTTTCCGGGTTGGACTCAAGACGATAGTTACCGGATAAGGCGCAGCGGTCGGACT
GAACGGGGGGTTCGTGCATACAGTCCAGCTTGGAGCGAACTGCCTACCCGGAACTGAGTGTCAGG
CGTGGAATGAGACAAACGCGGCCATAACAGCGGAATGACACCGGTAAACCGAAAGGCAGGAACA
GGAGAGCGCACGAGGGAGCCGCCAGGGGGAAACGCCTGGTATCTTTATAGTCCTGTCGGGTTTCG
CCACCACTGATTTGAGCGTCAGATTTCGTGATGCTTGTCAGGGGGGCGGAGCCTATGGAAAAACG
GCTTTGCCGCGGCCCTCTCACTTCCCTGTTAAGTATCTTCCTGGCATCTTCCAGGAAATCTCCGCCC
CGTTCGTAAGCCATTTCCGCTCGCCGCAGTCGAACGACCGAGCGTAGCGAGTCAGTGAGCGAGGA
AGCGGAATATATCC
oriV-SEQ ID NO: 23
AGCGGGCCGGGAGGGTTCGAGAAGGGGGGGCACCCCCCTTCGGCGTGCGCGGTCACGCG
CCAGGGCGCAGCCCTGGTTAAAAACAAGGTTTATAAATATTGGTTTAAAAGCAGGTTAAAAGACA
GGTTAGCGGTGGCCGAAAAACGGGCGGAAACCCTTGCAAATGCTGGATTTTCTGCCTGTGGACAG
CCCCTCAAATGTCAATAGGTGCGCCCCTCATCTGTCATCACTCTGCCCCTCAAGTGTCAAGGATCG
CGCCCCTCATCTGTCAGTAGTCGCGCCCCTCAAGTGTCAATACCGCAGGGCACTTATCCCCAGGCT
TGTCCACATCATCTGTGGGAAACTCGCGTAAAATCAGGCGTTTTCGCCGATTTGCGAGGCTGGCCA
GCTCCACGTCGCCGGCCGAAATCGAGCCTGCCCCTCATCTGTCAACGCCGCGCCGGGTGAGTCGGC
CCCTCAAGTGTCAACGTCCGCCCCTCATCTGTCAGTGAGGGCCAAGTTTTCCGCGTGGTATCCACA
ACGCCGGCGGCCGCGGTGTCTCGCACACGGCTTCGACGGCGTTTCTGGCGCGTTTGCAGGGCCATA
GACGGCCGCCAGCCCAGCGGCGAGGGCAACCAGCCCGGTGAGCGTCGGAAAGGCGCTGGAAGCC
CCGTAGCGACGCGGAGAGGGGCGAGACAAGCCAAGGGCGCAGGCTCGATGCGCAGCACGACATA
GCCGGTTCTCGCAAGGACGAGAATTTCCCTGCGGTGCCCCTCAAGTGTCAATGAAAGTTTCCAACG
CGAGCCATTCGCGAGAGCCTTGAGTCCACGCTAGATCTATCTCA Requires trfA protein for
replication
pBBR1 origin of replication-SEQ ID NO: 24
CTACGGGCTTGCTCTCCGGGCTTCGCCCTGCGCGGTCGCTGCGCTCCCTTGCCAGCCCGTG
GATATGTGGACGATGGCCGCGAGCGGCCACCGGCTGGCTCGCTTCGCTCGGCCCGTGGACAACCC
TGCTGGACAAGCTGATGGACAGGCTGCGCCTGCCCACGAGCTTGACCACAGGGATTGCCCACCGG
CTACCCAGCCTTCGACCACATACCCACCGGCTCCAACTGCGCGGCCTGCGGCCTTGCCCCATCAAT
TTTTTTAATTTTCTCTGGGGAAAAGCCTCCGGCCTGCGGCCTGCGCGCTTCGCTTGCCGGTTGGACA
CCAAGTGGAAGGCGGGTCAAGGCTCGCGCAGCGACCGCGCAGCGGCTTGGCCTTGACGCGCCTGG
AACGACCCAAGCCTATGCGAGTGGGGGCAGTCGAAGGCGAAGCCCGCCCGCCTGCCCCCCGAGCC
TCACGGCGGCGAGTGCGGGGGTTCCAAGGGGGCAGCGCCACCTTGGGCAAGGCCGAAGGCCGCG
CAGTCGATCAACAAGCCCCGGAGGGGCCACTTTTTGCCGGAGGGGGAGCCGCGCCGAAGGCGTGG
GGGAACCCCGCAGGGGTGCCCTTCTTTGGGCACCAAAGAACTAGATATAGGGCGAAATGCGAAAG
ACTTAAAAATCAACAACTTAAAAAAGGGGGGTACGCAACAGCTCATTGCGGCACCCCCCGCAATA
GCTCATTGCGTAGGTTAAAGAAAATCTGTAATTGACTGCCACTTTTACGCAACGCATAATTGTTGT
CGCGCTGCCGAAAAGTTGCAGCTGATTGCGCATGGTGCCGCAACCGTGCGGCACCCTACCGCATG
GAGATAAGCATGGCCACGCAGTCCAGAGAAATCGGCATTCAAGCCAAGAACAAGCCCGGTCACTG
GGTGCAAACGGAACGCAAAGCGCATGAGGCGTGGGCCGGGCTTATTGCGAGGAAACCCACGGCG
GCAATGCTGCTGCATCACCTCGTGGCGCAGATGGGCCACCAGAACGCCGTGGTGGTCAGCCAGAA
GACACTTTCCAAGCTCATCGGACGTTCTTTGCGGACGGTCCAATACGCAGTCAAGGACTTGGTGGC
CGAGCGCTGGATCTCCGTCGTGAAGCTCAACGGCCCCGGCACCGTGTCGGCCTACGTGGTCAATGA
CCGCGTGGCGTGGGGCCAGCCCCGCGACCAGTTGCGCCTGTCGGTGTTCAGTGCCGCCGTGGTGGT
TGATCACGACGACCAGGACGAATCGCTGTTGGGGCATGGCGACCTGCGCCGCATCCCGACCCTGT
ATCCGGGCGAGCAGCAACTACCGACCGGCCCCGGCGAGGAGCCGCCCAGCCAGCCCGGCATTCCG
GGCATGGAACCAGACCTGCCAGCCTTGACCGAAACGGAGGAATGGGAACGGCGCGGGCAGCAGC
GCCTGCCGATGCCCGATGAGCCGTGTTTTCTGGACGATGGCGAGCCGTTGGAGCCGCCGACACGG
GTCACGCTGCCGCGCCGGTAG Includes coding sequence of required replication protein
RSF1010 plasmid backbone-SEQ ID NO: 25
GCTCGACCAGGCGTACGCTTATGGGTGCCTTTCCGCAGCTTGGAACGCGGATGGAGAAGA
GGAGCAACGCGATCTAGCTATCGCGGCCGCGATCAAGCAGGTGCGACAGACGTCATACTAGATAT
CAAGCGACTTCTCCTATCCCCTGGGAACACATCAATCTCACCGGAGAATATCGCTGGCCAAAGCCT
TAGCGTAGGATTCCGCCCCTTCCCGCAAACGACCCCAAACAGGAAACGCAGCTGAAACGGGAAGC
TCAACACCCACTGACGCATGGGTTGTTCAGGCAGTACTTCATCAACCAGCAAGGCGGCACTTTCGG
CCATCCGCCGCGCCCCACAGCTCGGGCAGAAACCGCGACGCTTACAGCTGAAAGCGACCAGGTGC
TCGGCGTGGCAAGACTCGCAGCGAACCCGTAGAAAGCCATGCTCCAGCCGCCCGCATTGGAGAAA
TTCTTCAAATTCCCGTTGCACATAGCCCGGCAATTCCTTTCCCTGCTCTGCCATAAGCGCAGCGAAT
GCCGGGTAATACTCGTCAACGATCTGATAGAGAAGGGTTTGCTCGGGTCGGTGGCTCTGGTAACG
ACCAGTATCCCGATCCCGGCTGGCCGTCCTGGCCGCCACATGAGGCATGTTCCGCGTCCTTGCAAT
ACTGTGTTTACATACAGTCTATCGCTTAGCGGAAAGTTCTTTTACCCTCAGCCGAAATGCCTGCCGT
TGCTAGACATTGCCAGCCAGTGCCCGTCACTCCCGTACTAACTGTCACGAACCCCTGCAATAACTG
TCACGCCCCCCTGCAATAACTGTCACGAACCCCTGCAATAACTGTCACGCCCCCAAACCTGCAAAC
CCAGCAGGGGCGGGGGCTGGCGGGGTGTTGGAAAAATCCATCCATGATTATCTAAGAATAATCCA
CTAGGCGCGGTTATCAGCGCCCTTGTGGGGCGCTGCTGCCCTTGCCCAATATGCCCGGCCAGAGGC
CGGATAGCTGGTCTATTCGCTGCGCTAGGCTACACACCGCCCCACCGCTGCGCGGCAGGGGGAAA
GGCGGGCAAAGCCCGCTAAACCCCACACCAAACCCCGCAGAAATACGCTGGAGCGCTTTTAGCCG
CTTTAGCGGCCTTTCCCCCTACCCGAAGGGTGGGGGCGCGTGTGCAGCCCCGCAGGGCCTGTCTCG
GTCGATCATTCAGCCCGGCTCATCCTTCTGGCGTGGCGGCAGACCGAACAAGGCGCGGTCGTGGTC
GCGTTCAAGGTACGCATCCATTGCCGCCATGAGCCGATCCTCCGGCCACTCGCTGCTGTTCACCTT
GGCCAAAATCATGGCCCCCACCAGCACCTTGCGCCTTGTTTCGTTCTTGCGCTCTTGCTGCTGTTCC
CTTGCCCGCACCCGCTGAATTTCGGCATTGATTCGCGCTCGTTGTTCTTCGAGCTTGGCCAGCCGAT
CCGCCGCCTTGTTGCTCCCCTTAACCATCTTGACACCCCATTGTTAATGTGCTGTCTCGTAGGCTAT
CATGGAGGCACAGCGGCGGCAATCCCGACCCTACTTTGTAGGGGAGGGCGCACTTACCGGTTTCTC
TTCGAGAAACTGGCCTAACGGCCACCCTTCGGGCGGTGCGCTCTCCGAGGGCCATTGCATGGAGCC
GAAAAGCAAAAGCAACAGCGAGGCAGCATGGCGATTTATCACCTTACGGCGAAAACCGGCAGCA
GGTCGGGCGGCCAATCGGCCAGGGCCAAGGCCGACTACATCCAGCGCGAAGGCAAGTATGCCCGC
GACATGGATGAAGTCTTGCACGCCGAATCCGGGCACATGCCGGAGTTCGTCGAGCGGCCCGCCGA
CTACTGGGATGCTGCCGACCTGTATGAACGCGCCAATGGGCGGCTGTTCAAGGAGGTCGAATTTGC
CCTGCCGGTCGAGCTGACCCTCGACCAGCAGAAGGCGCTGGCGTCCGAGTTCGCCCAGCACCTGA
CCGGTGCCGAGCGCCTGCCGTATACGCTGGCCATCCATGCCGGTGGCGGCGAGAACCCGCACTGC
CACCTGATGATCTCCGAGCGGATCAATGACGGCATCGAGCGGCCCGCCGCTCAGTGGTTCAAGCG
GTACAACGGCAAGACCCCGGAGAAGGGCGGGGCACAGAAGACCGAAGCGCTCAAGCCCAAGGCA
TGGCTTGAGCAGACCCGCGAGGCATGGGCCGACCATGCCAACCGGGCATTAGAGCGGGCTGGCCA
CGACGCCCGCATTGACCACAGAACACTTGAGGCGCAGGGCATCGAGCGCCTGCCCGGTGTTCACC
TGGGGCCGAACGTGGTGGAGATGGAAGGCCGGGGCATCCGCACCGACCGGGCAGACGTGGCCCT
GAACATCGACACCGCCAACGCCCAGATCATCGACTTACAGGAATACCGGGAGGCAATAGACCATG
AACGCAATCGACAGAGTGAAGAAATCCAGAGGCATCAACGAGTTAGCGGAGCAGATCGAACCGC
TGGCCCAGAGCATGGCGACACTGGCCGACGAAGCCCGGCAGGTCATGAGCCAGACCCAGCAGGCC
AGCGAGGCGCAGGCGGCGGAGTGGCTGAAAGCCCAGCGCCAGACAGGGGCGGCATGGGTGGAGC
TGGCCAAAGAGTTGCGGGAGGTAGCCGCCGAGGTGAGCAGCGCCGCGCAGAGCGCCCGGAGCGC
GTCGCGGGGGTGGCACTGGAAGCTATGGCTAACCGTGATGCTGGCTTCCATGATGCCTACGGTGGT
GCTGCTGATCGCATCGTTGCTCTTGCTCGACCTGACGCCACTGACAACCGAGGACGGCTCGATCTG
GCTGCGCTTGGTGGCCCGATGAAGAACGACAGGACTTTGCAGGCCATAGGCCGACAGCTCAAGGC
CATGGGCTGTGAGCGCTTCGATATCGGCGTCAGGGACGCCACCACCGGCCAGATGATGAACCGGG
AATGGTCAGCCGCCGAAGTGCTCCAGAACACGCCATGGCTCAAGCGGATGAATGCCCAGGGCAAT
GACGTGTATATCAGGCCCGCCGAGCAGGAGCGGCATGGTCTGGTGCTGGTGGACGACCTCAGCGA
GTTTGACCTGGATGACATGAAAGCCGAGGGCCGGGAGCCTGCCCTGGTAGTGGAAACCAGCCCGA
AGAACTATCAGGCATGGGTCAAGGTGGCCGACGCCGCAGGCGGTGAACTTCGGGGGCAGATTGCC
CGGACGCTGGCCAGCGAGTACGACGCCGACCCGGCCAGCGCCGACAGCCGCCACTATGGCCGCTT
GGCGGGCTTCACCAACCGCAAGGACAAGCACACCACCCGCGCCGGTTATCAGCCGTGGGTGCTGC
TGCGTGAATCCAAGGGCAAGACCGCCACCGCTGGCCCGGCGCTGGTGCAGCAGGCTGGCCAGCAG
ATCGAGCAGGCCCAGCGGCAGCAGGAGAAGGCCCGCAGGCTGGCCAGCCTCGAACTGCCCGAGC
GGCAGCTTAGCCGCCACCGGCGCACGGCGCTGGACGAGTACCGCAGCGAGATGGCCGGGCTGGTC
AAGCGCTTCGGTGATGACCTCAGCAAGTGCGACTTTATCGCCGCGCAGAAGCTGGCCAGCCGGGG
CCGCAGTGCCGAGGAAATCGGCAAGGCCATGGCCGAGGCCAGCCCAGCGCTGGCAGAGCGCAAG
CCCGGCCACGAAGCGGATTACATCGAGCGCACCGTCAGCAAGGTCATGGGTCTGCCCAGCGTCCA
GCTTGCGCGGGCCGAGCTGGCACGGGCACCGGCACCCCGCCAGCGAGGCATGGACAGGGGCGGG
CCAGATTTCAGCATGTAGTGCTTGCGTTGGTACTCACGCCTGTTATACTATGAGTACTCACGCACA
GAAGGGGGTTTTATGGAATACGAAAAAAGCGCTTCAGGGTCGGTCTACCTGATCAAAAGTGACAA
GGGCTATTGGTTGCCCGGTGGCTTTGGTTATACGTCAAACAAGGCCGAGGCTGGCCGCTTTTCAGT
CGCTGATATGGCCAGCCTTAACCTTGACGGCTGCACCTTGTCCTTGTTCCGCGAAGACAAGCCTTT
CGGCCCCGGCAAGTTTCTCGGTGACTGATATGAAAGACCAAAAGGACAAGCAGACCGGCGACCTG
CTGGCCAGCCCTGACGCTGTACGCCAAGCGCGATATGCCGAGCGCATGAAGGCCAAAGGGATGCG
TCAGCGCAAGTTCTGGCTGACCGACGACGAATACGAGGCGCTGCGCGAGTGCCTGGAAGAACTCA
GAGCGGCGCAGGGCGGGGGTAGTGACCCCGCCAGCGCCTAACCACCAACTGCCTGCAAAGGAGG
CAATCAATGGCTACCCATAAGCCTATCAATATTCTGGAGGCGTTCGCAGCAGCGCCGCCACCGCTG
GACTACGTTTTGCCCAACATGGTGGCCGGTACGGTCGGGGCGCTGGTGTCGCCCGGTGGTGCCGGT
AAATCCATGCTGGCCCTGCAACTGGCCGCACAGATTGCAGGCGGGCCGGATCTGCTGGAGGTGGG
CGAACTGCCCACCGGCCCGGTGATCTACCTGCCCGCCGAAGACCCGCCCACCGCCATTCATCACCG
CCTGCACGCCCTTGGGGCGCACCTCAGCGCCGAGGAACGGCAAGCCGTGGCTGACGGCCTGCTGA
TCCAGCCGCTGATCGGCAGCCTGCCCAACATCATGGCCCCGGAGTGGTTCGACGGCCTCAAGCGC
GCCGCCGAGGGCCGCCGCCTGATGGTGCTGGACACGCTGCGCCGGTTCCACATCGAGGAAGAAAA
CGCCAGCGGCCCCATGGCCCAGGTCATCGGTCGCATGGAGGCCATCGCCGCCGATACCGGGTGCT
CTATCGTGTTCCTGCACCATGCCAGCAAGGGCGCGGCCATGATGGGCGCAGGCGACCAGCAGCAG
GCCAGCCGGGGCAGCTCGGTACTGGTCGATAACATCCGCTGGCAGTCCTACCTGTCGAGCATGACC
AGCGCCGAGGCCGAGGAATGGGGTGTGGACGACGACCAGCGCCGGTTCTTCGTCCGCTTCGGTGT
GAGCAAGGCCAACTATGGCGCACCGTTCGCTGATCGGTGGTTCAGGCGGCATGACGGCGGGGTGC
TCAAGCCCGCCGTGCTGGAGAGGCAGCGCAAGAGCAAGGGGGTGCCCCGTGGTGAAGCCTAAGA
ACAAGCACAGCCTCAGCCACGTCCGGCACGACCCGGCGCACTGTCTGGCCCCCGGCCTGTTCCGTG
CCCTCAAGCGGGGCGAGCGCAAGCGCAGCAAGCTGGACGTGACGTATGACTACGGCGACGGCAA
GCGGATCGAGTTCAGCGGCCCGGAGCCGCTGGGCGCTGATGATCTGCGCATCCTGCAAGGGCTGG
TGGCCATGGCTGGGCCTAATGGCCTAGTGCTTGGCCCGGAACCCAAGACCGAAGGCGGACGGCAG
CTCCGGCTGTTCCTGGAACCCAAGTGGGAGGCCGTCACCGCTGAATGCCATGTGGTCAAAGGTAG
CTATCGGGCGCTGGCAAAGGAAATCGGGGCAGAGGTCGATAGTGGTGGGGCGCTCAAGCACATAC
AGGACTGCATCGAGCGCCTTTGGAAGGTATCCATCATCGCCCAGAATGGCCGCAAGCGGCAGGGG
TTTCGGCTGCTGTCGGAGTACGCCAGCGACGAGGCGGACGGGCGCCTGTACGTGGCCCTGAACCC
CTTGATCGCGCAGGCCGTCATGGGTGGCGGCCAGCATGTGCGCATCAGCATGGACGAGGTGCGGG
CGCTGGACAGCGAAACCGCCCGCCTGCTGCACCAGCGGCTGTGTGGCTGGATCGACCCCGGCAAA
ACCGGCAAGGCTTCCATAGATACCTTGTGCGGCTATGTCTGGCCGTCAGAGGCCAGTGGTTCGACC
ATGCGCAAGCGCCGCCAGCGGGTGCGCGAGGCGTTGCCGGAGCTGGTCGCGCTGGGCTGGACGGT
AACCGAGTTCGCGGCGGGCAAGTACGACATCACCCGGCCCAAGGCGGCAGGCTGACCCCCCCCAC
TCTATTGTAAACAAGACATTTTTATCTTTTATATTCAATGGCTTATTTTCCTGCTAATTGGTAATACC
ATGAAAAATACCATGCTCAGAAAAGGCTTAACAATATTTTGAAAAATTGCCTACTGAGCGCTGCC
GCACAGCTCCATAGGCC Includes genes for mobilization proteins A, B, C and replication proteins
A, B, C
RCR-SEQ ID NO: 26
TCCGCCGCCCTAGACCTAGTGTCATTTTATTTCCCCCGTTTCAGCATCAAGAACCTTTGCAT
AACTTGCTCTATATCCACACTGATAATTGCCCTCAAACCATAATCTAAAGGCGCTAGAGTTTGTTG
AAACAATATCTTTTACATCATTCGTATTTAAAATTCCAAACTCCGCTCCCCTAAGGCGAATAAAAG
CCATTAAATCTTTTGTATTTACCAAATTATAGTCATCCACTATATCTAAGAGTAAATTCTTCAATTC
TCTTTTTTGGCTTTCATCAAGTGTTATATAGCGGTCAATATCAAAATCATTAATGTTCAAAATATCT
TTTTTGTCGTATATATGTTTATTCTTAGCAATAGCGTCCTTTGATTCATGAGTCAAATATTCATATG
AACCTTTGATATAATCAAGTATCTCAACATGAGCAACTGAACTATTCCCCAATTTTCGCTTAATCTT
GTTCCTAACGCTTTCTATTGTTACAGGATTTCGTGCAATATATATAACGTGATAGTGTGGTTTTTTA
TAGTGCTTTCCATTTCGTATAACATCACTACTATTCCATGTATCTTTATCTTTTTTTTCGTCCATATC
GTGTAAAGGACTGACAGCCATAGATACGCCCAAACTCTCTAATTTTTCCTTCCAATCATTAGGAAT
TGAGTCAGGATATAATAAAAATCCAAAATTTCTAGCTTTAGTATTTTTAATAGCCATGATATAATT
ACCTTATCAAAAACAAGTAGCGAAAACTCGTATCCTTCTAAAAACGCGAGCTTTCGCTTATTTTTTT
TGTTCTGATTCCTTTCTTGCATATTCTTCTATAGCTAACGCCGCAACCGCAGATTTTGAAAAACCTT
TTTGTTTCGCCATATCTGTTAATTTTTTATCTTGCTCTTTTGTCAGAGAAATCATAACTCTTTTTTTC
GATTCTGAAATCACCATTTAAAAAACTCCAATCAAATAATTTTATAAAGTTAGTGTATCACTTTGT
AATCATAAAAACAACAATAAAGCTACTTAAATATAGATTTATAAAAAACGTTGGCGAAAACGTTG
GCGATTCGTTGGCGATTGAAAAACCCCTTAAACCCTTGAGCCAGTTGGGATAGAGCGTTTTTGGCA
CAAAAATTGGCACTCGGCACTTAATGGGGGGTCGTAGTACGGAAGCAAAATTCGCTTCCTTTCCCC
CCATTTTTTTCCAAATTCCAAATTTTTTTCAAAAATTTTCCAGCGCTACCGCTCGGCAAAATTGCAA
GCAATTTTTAAAATCAAACCCATGAGGGAATTTCATTCCCTCATACTCCCTTGAGCCTCCTCCAACC
GAAATAGAAGGGCGCTGCGCTTATTATTTCATTCAGTCATCGGCTTTCATAATCTAACAGACAACA
TCTTCGCTGCAAAGCCACGCTACGCTCAAGGGCTTTTACGCTACGATAACGCCTGTTTTAACGATT
ATGCCGATAACTAAACGAAATAAACGCTAAAACGTCTCAGAAACGATTTTGAGACGTTTTAATAA
AAAATCGCCTAGTGC
Transposon inverted repeat sequences
Himar ACAGGTTGGATGATAAGTCCCCGGTCT (SEQ ID NO: 9)
Tn5 CTGTCTCTTATACACATCT (SEQ ID NO: 10)
Regulatory sequences (5′ UTRs, incl. promoter and RBS)
SEQ ID NO: 1
GATTGCATTAGGTTTTAGTTTCTTGTATAATGCTTAATGTTGGTCACTGACAGGCTACGAT
ACGGAAGGTTGCTCACGCCCGGCCCCTTTGCCATGGCTAGTGTGTGGAAATTTCCGAGGAGCAAGT
CTATTTCCAAAAATGGGCGAAAAAGGAGGTAATACA From Bacillus cellulosilyticus
SEQ ID NO: 2
GGGAGAGCTTCAACGGCGCTTCTACCCATTTGCTTGGAAAGGATGAGGAGCAGGAAGAAA
TTCCGTCCCCAATGCGACGGCCCTTTACATCCATGTTGTTTGATAGTATAATGGATACGGATTGACC
AAATTGTTCATTTAGTCAGTTTGAAGGATGAGGAGT From Geobacillus sp.
SEQ ID NO: 3
GTGAAGGATACGGCTGCGGCACTTCGACATCGCCCCATGTGGCGGCTTTGAACTGGGCTT
ATGAAACGCGTTCACAACCTTTTTTGACCATCGGCGCGAACGTGGTATCATGCGTTCAGCTTTTGC
CCATACATACTACGTGCTCAATCTAGGAGGATTTCATAC From Eggerthella lenta
SEQ ID NO: 4
CTCTAGAGTAGTAGATTATTTTAGGAATTTAGATGTTTTGTATGAAATAGATGCTTCGTAT
GGAATTAATGAAATTTTTAGTCAGGTAAAAAAGGTAATAGGAGAATATT From Segmented
Filamentous Bacteria (SFB)
SEQ ID NO: 5
GTTTTAAATGATGAAAAGAAATATTTAGGGAAGATTGTTTCGACGCGAATTGTTGATCTGG
AAAATGATCACCTTATCGGACAAGCTTTAAAATAGGAGGATATAAAAAT From Segmented
Filamentous Bacteria (SFB)
SEQ ID NO: 6
ATAAGGATTCTTTAAAGAGAGATATAGTTATGTCAAAGACTGTAGAATTTTTAGTAAATCA
AAATAAAAAAAGAGGTATTAAATAGAGTGTATTTTAAAGGAGGAGACTT From Segmented
Filamentous Bacteria (SFB)
SEQ ID NO: 7
AAACACCAATAAAATTAGAATATTTAGGAGCGACTTTAAAAAAGTTTAATAAGAATTGTT
TATGAGATATTTTTATTATATTTAAACTCAATTTAAAGTAGGGAGAATAG From Segmented
Filamentous Bacteria (SFB)
SEQ ID NO: 8
GCAAGTGTTCAAGAAGTTATTAAGTCGGGAGTGCAGTCGAAGTGGGCAAGTTGAAAAATT
CACAAAAATGTGGTATAATATCTTTGTTCATTAGAGCGATAAACTTGAATTTGAGAGGGAACTTAG
From Clostridium perfringens
Primers for PCR validation of transconjugants
16S forward AGAGTTTGATCATGGCTCAG (SEQ ID NO: 27)
16S reverse CGGTTACCTTGTTACGACTT (SEQ ID NO: 28)
GFP validation primer forward ATGCGTAAAGGCGAAGAGC (SEQ ID NO: 29)
GFP validation primer reverse TTATTTGTACAGTTCATCCATACCATG (SEQ ID NO: 30)
Beta-lactamase validation primer forward ATGAGTATTCAACATTTCCGTGTC (SEQ ID NO: 31)
Beta-lactamase validation primer reverse TTACCAATGCTTAATCAGTGAGGC (SEQ ID NO: 32)
pGT-B backbone validation primer forward CTGCGCAACCCAAGTGCTAC (SEQ ID NO: 33)
pGT-B backbone validation primer reverse CAGTCCAGAGAAATCGGCATTCA (SEQ ID NO: 34)
pGT-Ah backbone validation primer forward ATGGAAAAAAAGGAATTTCGTGTTTTG (SEQ ID
NO: 35)
pGT-Ah backbone validation primer reverse TTATTCAACATAGTTCCCTTCAAGAGC (SEQ ID NO: 36)
CarbR internal forward primer CCGAAGAACGTTTTCCAATGATGAG (SEQ ID NO: 37)
GFP internal reverse primerTGATTGTCTGGCAGCAGAAC (SEQ ID NO: 38)
catP (chlor resistance) validation primer forward GCAAGTGTTCAAGAAGTTATTAAGTC (SEQ ID NO:
39)
catP (chlor resistance) validation primer reverse TTAACTATTTATCAATTCCTGCAATTCG (SEQ ID
NO: 40)
tetQ (tet resistance) internal forward primer TGGAAGAACGATTTTCCGTAAAGGT (SEQ ID NO: 41)

TABLE 4
List of isolated transconjugant strains
Strains are grouped by the mouse cohort they were isolated from and the vector library used
in the study.
All family-level assignments were made using the RDP classifier with confidence >0.89.
Taconic mice in situ conjugations

| | | | Genus-level | | |
|---|---|---|---|---|---|
| Vector | | | assignment | Vector | Antibiotic |
| library | Family | Genus | confidence | received | resistance |
| pGT-L6 | Erysipelotrichaceae | Erysipelotrichaceae | 1 | pGT-Ah | carb |
| | (Clostridium XVIII) | incertae sedis | | | |
| | Bacteroidaceae | Bacteroides | 1 | pGT-Ah | carb |
| | Enterobacteriaceae | Proteus | 1 | pGT-Ah | carb |
| | Enterobacteriaceae | Citrobacter | 1 | PGT-Ah | carb |
| | Enterococcaceae | Enterococcus | 1 | PGT-Ah | carb |
| | Lachnospiraceae | Hungatella | 0.72 | PGT-Ah | carb |
| | Lachnospiraceae | Clostridium XIVa | 1 | PGT-Ah | carb |
| | Lachnospiraceae | Anaerostipes | 1 | PGT-Ah | carb |
| | Lachnospiraceae | Moryella | 0.19 | PGT-Ah | carb |
| | Lachnospiraceae | Blautia | 1 | PGT-Ah | carb |
| | Lactobacillaceae | Lactobacillus | 1 | PGT-Ah | carb |
| | Peptostreptococcaceae | Clostridium XI | 1 | pGT-Ah | carb |
| pGT-L3 | Coriobacteriaceae | Epperthella | 1 | pGT-S | tet |
| | Enterobacteriaceae | Cosenzaea | 0.73 | pGT-S | tet |
| | Enterobacteriaceae | Proteus | 1 | pGT-S | tet |
| | Enterococcaceae | Enterococcus | 1 | pGT-S | carb |
| | Lachnospiraceae | Lactonifactor | 0.7 | pGT-S | tet |
| | Lachnospiraceae | Clostridium XIVa | 1 | pGT-S | carb |
| | Lachnospiraceae | Hungatella | 0.71 | pGT-S | tet |
| | Lachnospiraceae | Clostridium XIVa | 1 | pGT-S | tet |
| | Lachnospiraceae | Blautia | 1 | pGT-S | tet |
| | Lachnospiraceae | Robinsoniella | 0.42 | pGT-S | tet |
| | Lachnospiraceae | Eisenberpiella | 0.99 | pGT-S | tet |
| | Lactobacillaceae | Lactobacillus | 0.89 | pGT-S | tet |

Charles River mice in situ conjugations

| | | | | | |
|---|---|---|---|---|---|
| pGT-L6 | Bacteroidaceae | Bacteroides | 1 | pGT-Ah | carb |
| | Enterococcaceae | Enterococcus | 1 | PGT-Ah | carb |
| | Lactobacillaceae | Lactobacillus | 1 | PGT-Ah | carb |
| | Porphyromonadaceae | Parabacteroides | 1 | pGT-Ah | carb |

In vitro conjugations

| | | | | | |
|---|---|---|---|---|---|
| pGT-L7 | Enterobacteriaceae | Proteus | 1 | pGT-Ah | carb |
| | Enterococcaceae | Enterococcus | 1 | pGT-Ah | carb |
| | Enterobacteriaceae | Escherichia | 1 | PGT-Ah | carb |
| | Lactobacillaceae | Lactobacillus | 1 | PGT-Ah | carb |
| | Bacillaceae | Bacillus | 1 | PGT-Ah | carb |
| pGT-L3 | Enterobacteriaceae | Escherichia | 1 | pGT-S | carb |
| | Enterococcaceae | Enterococcus | 1 | pGT-S | carb |
| | Enterobacteriaceae | Proteus | 1 | pGT-S | carb |
| pGT-L5 | Enterobacteriaceae | Cosenzae | 0.89 | PGT-Ah | chi |
| | Enterobacteriaceae | Proteus | 1 | pGT-Ah | chi |
| | Burkholderiaceae | Cupriavidus | 1 | pGT-Ah | chi |
| pGT-L4 | Enterobacteriaceae | Escherichia | 1 | PGT-Ah | carb |
| | Enterobacteriaceae | Proteus | 1 | PGT-Ah | carb |
| | Enterobacteriaceae | Escherichia | 1 | pGT-B | carb |

Many modifications and variations of this invention can be made without departing from its spirit and scope, as will be apparent to those skilled in the art. The invention is defined by the terms of the appended claims, along with the full scope of equivalents to which such claims are entitled. The specific embodiments described herein, including the following examples, are offered by way of example only, and do not by their details limit the scope of the invention.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GenelD entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

1. A method for altering a microbiome of a subject, comprising the steps of:
(a) providing a donor bacterial strain, wherein the donor bacterial strain comprises a genomically integrated conjugation system or an episomal system, a fluorophore gene and is optionally auxotropic for at least one compound;
(b) introducing a plasmid vector to the donor bacterial strain, wherein the plasmid vector comprises an origin of replication (oriR), an origin of transfer (oriT), an antibiotic selection gene, and sfGFP gene; or the plasmid vector comprises an OriR, and OriT, a Himar transposon comprising an antibiotic selection gene, sfGFP gene and a Himar transposase gene;
(c) selecting recipient bacterial strains that had incorporated the plasmid vector by antibiotic selection or Fluorescence Activated Cell Sorting (FACS); and,
(d) optionally recolonizing the subject with recipient bacteria from step (c).

2. The method of claim 1, further comprising the steps of:
(i) isolating gut bacteria from the subject to provide a recipient bacterial strain after step (b); and,
(ii) mixing the donor and recipient bacterial strains.

3. (canceled)

4. The method of claim 1, wherein the conjugation system is RP4, R1, F conjugation or pKM101.

5. The method of claim 1, wherein the origin of replication (oriR) is selected from the group consisting of pBBR1, OriV, R6K, p15A, pBI143, IncP, IncX, IncF, IncB, Col and RS1010.

6. The method of claim 1, wherein the origin of transfer (oriT) is Rk2 or F.

7-8. (canceled)

9. The method of claim 1, wherein the donor bacterial strain is Escherichia coli, or a strain of Shigella.

10-11. (canceled)

12. The method of claim 1, wherein the recipient bacterial strain is of a phyla selected from the group consisting of Bacteriodetes, Proteobacteria, Fusobacteria, Actinobacteria, Deferribacteres, Tenericutes, Planctomycetes, and Firmicutes.

13-15. (canceled)

16. A bacterial cell comprising a genomically integrated or episomal conjugation system into which a plasmid vector has been introduced, the plasmid vector comprising
at least one origin of replication (oriR) sequence;
an origin of transfer sequence;
a payload sequence of interest;
a regulatory sequence linked with the payload sequence so as to control expression of the payload sequence of interest; and
optionally, a transposase sequence and a first and second transposon-associated recognition sequence positioned upstream and downstream of the payload sequence, respectively, wherein the transposase comprises Himar transposase.

17. (canceled)

18. The bacterial cell of claim 16, wherein the at least one oriR sequence comprises a donor oriR and a recipient oriR.

19. (canceled)

20. The bacterial cell of claim 16, wherein the origin of transfer sequence is RK2 or F.

21. The bacterial cell of claim 16, wherein the conjugation system comprises a transfer (tra) sequence.

22. The bacterial cell of claim 21, wherein the tra sequence comprises at least a portion of RK2.

23. (canceled)

24. The bacterial cell of claim 16, wherein the regulatory sequence is active in one or more bacterial species.

25-34. (canceled)

35. The bacterial cell of claim 16, wherein the transposon-associated recognition sequence comprises SEQ ID No. 9 or SEQ ID NO. 10, or sequences possessing at least 80, 85, 90, 95 or 99 percent identity therewith.

36. A transconjugant bacterial cell that has received a payload sequence by conjugation with the bacterial cell of claim 16, wherein the isolated recipient bacterial cell is capable of conjugation and transfer of the payload sequence interest to another bacterial cell.

37-38. (canceled)

39. The transconjugant bacterial cell of claim 36, wherein the payload sequence of interest was received in situ or in vitro.

40. The transconjugant bacterial cell of claim 36,, wherein the transconjugant bacterial cell is of a different strain or species respective to the bacterial cell of claim 16.

41. A method of altering a microbiome comprising contacting at least one cell of the microbiome with a bacterial cell of claim 16.

42. The method of 41, wherein the microbiome is in or on a subject.

43. The method of claim 41, wherein the microbiome is a gut of the subject.

44-48. (canceled)


SEQUENCE LISTING (41 sequences)

SEQ ID NO: 1 - DNA, 163 bp, Bacillus cellulosilyticus
gattgcattaggttttagtttcttgtataatgcttaatgttggtcactgacaggctacgatacggaaggttgctcacgcccggcccctttgccatggctagtgtgtggaaatttccgaggagcaagtctatttccaaaaatgggcgaaaaaggaggtaataca

SEQ ID NO: 2 - DNA, 163 bp, Geobacillus sp.
gggagagcttcaacggcgcttctacccatttgcttggaaaggatgaggagcaggaagaaattccgtccccaatgcgacggccctttacatccatgttgtttgatagtataatggatacggattgaccaaattgttcatttagtcagtttgaaggatgaggagt

SEQ ID NO: 3 - DNA, 165 bp, Eggerthella lenta
gtgaaggatacggctgcggcacttcgacatcgccccatgtggcggctttgaactgggcttatgaaacgcgttcacaaccttttttgaccatcggcgcgaacgtggtatcatgcgttcagcttttgcccatacatactacgtgctcaatctaggaggatttcatac

SEQ ID NO: 4 - DNA, 110 bp, Segmented filamentous bacteria
ctctagagtagtagattattttaggaatttagatgttttgtatgaaatagatgcttcgtatggaattaatgaaatttttagtcaggtaaaaaaggtaataggagaatatt

SEQ ID NO: 5 - DNA, 110 bp, Segmented filamentous bacteria
gttttaaatgatgaaaagaaatatttagggaagattgtttcgacgcgaattgttgatctggaaaatgatcaccttatcggacaagctttaaaataggaggatataaaaat

SEQ ID NO: 6 - DNA, 110 bp, Segmented filamentous bacteria
ataaggattctttaaagagagatatagttatgtcaaagactgtagaatttttagtaaatcaaaataaaaaaagaggtattaaatagagtgtattttaaaggaggagactt

SEQ ID NO: 7 - DNA, 110 bp, Segmented filamentous bacteria
aaacaccaataaaattagaatatttaggagcgactttaaaaaagtttaataagaattgtttatgagatatttttattatatttaaactcaatttaaagtagggagaatag

SEQ ID NO: 8 - DNA, 126 bp, Clostridium perfringens
gcaagtgttcaagaagttattaagtcgggagtgcagtcgaagtgggcaagttgaaaaattcacaaaaatgtggtataatatctttgttcattagagcgataaacttgaatttgagagggaacttag

SEQ ID NO: 9 - DNA, 27 bp, Artificial Sequence
  Synthetic Himar
acaggttggatgataagtccccggtct

SEQ ID NO: 10 - DNA, 19 bp, Artificial Sequence
  Synthetic Tn5
ctgtctcttatacacatct

SEQ ID NO: 11 - protein, 18 aa, Artificial Sequence
  Synthetic linker
GGSGGSGGSGGSGGSGGS

SEQ ID NO: 12 - DNA, 27 bp, Artificial Sequence
  Synthetic modified Himar1 inverted repeat (IR) sequence
tagaccgggacttatcatccaacctgt

SEQ ID NO: 13 - DNA, 861 bp, Artificial Sequence
  Synthetic Beta-lactamase (carbenicillin/ampicillin resistance)
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 14 - DNA, 624 bp, Artificial Sequence
  Synthetic CatP (chloramphenicol resistance), Derived from pJIR750 plasmid from Clostridium perfringens
atggtatttgaaaaaattgataaaaatagttggaacagaaaagagtattttgaccactactttgcaagtgtaccttgtacatacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaa

SEQ ID NO: 15 - DNA, 1926 bp, Artificial Sequence
  Synthetic Tet (tetracycline resistance)
atgaatattataaatttaggaattcttgctcacattgatgcaggaaaaacttccgtaaccgagaatctgctgtttgccagtggagcaacggaaaagtgcggctgtgtggataatggtgacaccataacggactctatggatatagagaaacgtagaggaattactgttcgggcttctacgacatctattatctggaatggtgtgaaatgcaatatcattgacactccgggacacatggattttattgcggaagtggagcggacattcaaaatgcttgatggagcagtcctcatcttatccgcaaaggaaggcatacaagcgcagacaaagttgctgttcaatactttacagaagctgcaaatcccgacaattatatttatcaataagattgaccgagccggtgtgaatttggagcgtttgtatctggatataaaagcaaatctgtctcaagatgtcctgtttatgcaaaatgttgtcgatggatcggtttatccggtttgctcccaaacatatataaaggaagaatacaaagaatttgtatgcaaccatgacgacaatatattagaacgatatttggcggatagcgaaatttcaccggctgattattggaatacgataatcgctcttgtggcaaaagccaaagtctatccggtgctacatggatcagcaatgttcaatatcggtatcaatgagttgttggacgccatcacttcttttatacttcctccggcatcggtctcaaacagactttcatcttatctttataagatagagcatgaccccaaaggacataaaagaagttttctaaaaataattgacggaagtctgagacttcgagacgttgtaagaatcaacgattcggaaaaattcatcaagattaaaaatctaaaaactatcaatcagggcagagagataaatgttgatgaagtgggcgccaatgatatcgcgattgtagaggatatggatgattttcgaatcggaaattatttaggtgctgaaccttgtttgattcaaggattatcgcatcagcatcccgctctcaaatcctccgtccggccagacaggcccgaagagagaagcaaggtgatatccgctctgaatacattgtggattgaagacccgtctttgtccttttccataaactcatatagtgatgaattggaaatctcgttatatggtttaacccaaaaggaaatcatacagacattgctggaagaacgattttccgtaaaggtccattttgatgagatcaagactatatacaaagaacgacctgtaaaaaaggtcaataagattattcagatcgaagtgccgcccaacccttattgggccacaatagggctgactcttgaacccttaccgttagggacagggttgcaaatcgaaagtgacatctcctatggttatctgaaccattcttttcaaaatgccgtttttgaagggattcgtatgtcttgccaatccgggttacatggatgggaagtgactgatctgaaagtaacttttactcaagccgagtattatagcccggtaagtacacctgctgatttcagacagctgaccccttatgtcttcaggctggccttgcaacagtcaggtgtggacattctcgaaccgatgctctattttgagttgcagataccccaagcggcaagttccaaagctattacagatttgcaaaaaatgatgtctgagattgaagacatcagttgcaataatgagtggtgtcatattaaagggaaagttccattaaatacaagtaaagactatgcatcagaagtaagttcatacactaagggcttaggcatttttatggttaagccatgcgggtatcaaataacaaaaggcggttattctgataatatccgcatgaacgaaaaagataaacttttattcatgttccaaaaatcaatgtcatcaaaataa

SEQ ID NO: 16 - DNA, 1011 bp, Artificial Sequence
  Synthetic Spec (spectinomycin resistance)
atgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataa

SEQ ID NO: 17 - DNA, 810 bp, Artificial Sequence
  Synthetic Kan (kanamycin resistance)
atgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 18 - DNA, 735 bp, Artificial Sequence
  Synthetic ermG (erythromycin resistance)
atgaacaaagtaaatataaaagatagtcaaaattttattacttcaaaatatcacatagaaaaaataatgaattgcataagtttagatgaaaaagataacatctttgaaataggtgcagggaaaggtcattttactgctggattggtaaagagatgtaattttgtaacggcgatagaaattgattctaaattatgtgaggtaactcgtaataagctcttaaattatcctaactatcaaatagtaaatgatgatatactgaaatttacatttcctagccacaatccatataaaatatttggcagcataccttacaacataagcacaaatataattcgaaaaattgtttttgaaagttcagccacaataagttatttaatagtggaatatggttttgctaaaatgttattagatacaaacagatcactagcattgctgttaatggcagaggtagatatttctatattagcaaaaattcctaggtattatttccatccaaaacctaaagtggatagcacattaattgtattaaaaagaaagccagcaaaaatggcatttaaagagagaaaaaaatatgaaacttttgtaatgaaatgggttaacaaagagtacgaaaaactgtttacaaaaaatcaatttaataaagctttaaaacatgcgagaatatatgatataaacaatattagtttcgaacaatttgtatcgctatttaatagttataaaatatttaacggctaa

SEQ ID NO: 19 - DNA, 717 bp, Artificial Sequence
  Synthetic sfGFP
atgcgtaaaggcgaagagctgttcactggtttcgtcactattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccacatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcacatggttctgctggagttcgtaaccgcagcgggcatcacgcatggtatggatgaactgtacaaataa

SEQ ID NO: 20 - DNA, 660 bp, Artificial Sequence
  Synthetic Chlor (chloramphenicol resistance)
atggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaa

SEQ ID NO: 21 - DNA, 312 bp, Artificial Sequence
  Synthetic R6K origin of replication, Requires additional pir gene for replication
atccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttc

SEQ ID NO: 22 - DNA, 797 bp, Artificial Sequence
  Synthetic p15A origin of replication
aacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcc

SEQ ID NO: 23 - DNA, 823 bp, Artificial Sequence
  Synthetic oriV, Requires trfA protein for replication
agcgggccgggagggttcgagaagggggggcaccccccttcggcgtgcgcggtcacgcgccagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcatcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgtggtatccacaacgccggcggccgcggtgtctcgcacacggcttcgacggcgtttctggcgcgtttgcagggccatagacggccgccagcccagcggcgagggcaaccagcccggtgagcgtcggaaaggcgctggaagccccgtagcgacgcggagaggggcgagacaagccaagggcgcaggctcgatgcgcagcacgacatagccggttctcgcaaggacgagaatttccctgcggtgcccctcaagtgtcaatgaaagtttccaacgcgagccattcgcgagagccttgagtccacgctagatctatctca

SEQ ID NO: 24 - DNA, 1516 bp, Artificial Sequence
  Synthetic pBBR1 origin of replication, Includes coding sequence of required replication protein
ctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtag

SEQ ID NO: 25 - DNA, 6146 bp, Artificial Sequence
  Synthetic RSF1010 plasmid backbone, Includes genes for mobilization proteins A, B, C and replication proteins A, B, C
gctcgaccaggcgtacgcttatgggtgcctttccgcagcttggaacgcggatggagaagaggagcaacgcgatctagctatcgcggccgcgatcaagcaggtgcgacagacgtcatactagatatcaagcgacttctcctatcccctgggaacacatcaatctcaccggagaatatcgctggccaaagccttagcgtaggattccgccccttcccgcaaacgaccccaaacaggaaacgcagctgaaacgggaagctcaacacccactgacgcatgggttgttcaggcagtacttcatcaaccagcaaggcggcactttcggccatccgccgcgccccacagctcgggcagaaaccgcgacgcttacagctgaaagcgaccaggtgctcggcgtggcaagactcgcagcgaacccgtagaaagccatgctccagccgcccgcattggagaaattcttcaaattcccgttgcacatagcccggcaattcctttccctgctctgccataagcgcagcgaatgccgggtaatactcgtcaacgatctgatagagaagggtttgctcgggtcggtggctctggtaacgaccagtatcccgatcccggctggccgtcctggccgccacatgaggcatgttccgcgtccttgcaatactgtgtttacatacagtctatcgcttagcggaaagttcttttaccctcagccgaaatgcctgccgttgctagacattgccagccagtgcccgtcactcccgtactaactgtcacgaacccctgcaataactgtcacgcccccctgcaataactgtcacgaacccctgcaataactgtcacgcccccaaacctgcaaacccagcaggggcgggggctggcggggtgttggaaaaatccatccatgattatctaagaataatccactaggcgcggttatcagcgcccttgtggggcgctgctgcccttgcccaatatgcccggccagaggccggatagctggtctattcgctgcgctaggctacacaccgccccaccgctgcgcggcagggggaaaggcgggcaaagcccgctaaaccccacaccaaaccccgcagaaatacgctggagcgcttttagccgctttagcggcctttccccctacccgaagggtgggggcgcgtgtgcagccccgcagggcctgtctcggtcgatcattcagcccggctcatccttctggcgtggcggcagaccgaacaaggcgcggtcgtggtcgcgttcaaggtacgcatccattgccgccatgagccgatcctccggccactcgctgctgttcaccttggccaaaatcatggcccccaccagcaccttgcgccttgtttcgttcttgcgctcttgctgctgttcccttgcccgcacccgctgaatttcggcattgattcgcgctcgttgttcttcgagcttggccagccgatccgccgccttgttgctccccttaaccatcttgacaccccattgttaatgtgctgtctcgtaggctatcatggaggcacagcggcggcaatcccgaccctactttgtaggggagggcgcacttaccggtttctcttcgagaaactggcctaacggccacccttcgggcggtgcgctctccgagggccattgcatggagccgaaaagcaaaagcaacagcgaggcagcatggcgatttatcaccttacggcgaaaaccggcagcaggtcgggcggccaatcggccagggccaaggccgactacatccagcgcgaaggcaagtatgcccgcgacatggatgaagtcttgcacgccgaatccgggcacatgccggagttcgtcgagcggcccgccgactactgggatgctgccgacctgtatgaacgcgccaatgggcggctgttcaaggaggtcgaatttgccctgccggtcgagctgaccctcgaccagcagaaggcgctggcgtccgagttcgcccagcacctgaccggtgccgagcgcctgccgtatacgctggccatccatgccggtggcggcgagaacccgcactgccacctgatgatctccgagcggatcaatgacggcatcgagcggcccgccgctcagtggttcaagcggtacaacggcaagaccccggagaagggcggggcacagaagaccgaagcgctcaagcccaaggcatggcttgagcagacccgcgaggcatgggccgaccatgccaaccgggcattagagcgggctggccacgacgcccgcattgaccacagaacacttgaggcgcagggcatcgagcgcctgcccggtgttcacctggggccgaacgtggtggagatggaaggccggggcatccgcaccgaccgggcagacgtggccctgaacatcgacaccgccaacgcccagatcatcgacttacaggaataccgggaggcaatagaccatgaacgcaatcgacagagtgaagaaatccagaggcatcaacgagttagcggagcagatcgaaccgctggcccagagcatggcgacactggccgacgaagcccggcaggtcatgagccagacccagcaggccagcgaggcgcaggcggcggagtggctgaaagcccagcgccagacaggggcggcatgggtggagctggccaaagagttgcgggaggtagccgccgaggtgagcagcgccgcgcagagcgcccggagcgcgtcgcgggggtggcactggaagctatggctaaccgtgatgctggcttccatgatgcctacggtggtgctgctgatcgcatcgttgctcttgctcgacctgacgccactgacaaccgaggacggctcgatctggctgcgcttggtggcccgatgaagaacgacaggactttgcaggccataggccgacagctcaaggccatgggctgtgagcgcttcgatatcggcgtcagggacgccaccaccggccagatgatgaaccgggaatggtcagccgccgaagtgctccagaacacgccatggctcaagcggatgaatgcccagggcaatgacgtgtatatcaggcccgccgagcaggagcggcatggtctggtgctggtggacgacctcagcgagtttgacctggatgacatgaaagccgagggccgggagcctgccctggtagtggaaaccagcccgaagaactatcaggcatgggtcaaggtggccgacgccgcaggcggtgaacttcgggggcagattgcccggacgctggccagcgagtacgacgccgacccggccagcgccgacagccgccactatggccgcttggcgggcttcaccaaccgcaaggacaagcacaccacccgcgccggttatcagccgtgggtgctgctgcgtgaatccaagggcaagaccgccaccgctggcccggcgctggtgcagcaggctggccagcagatcgagcaggcccagcggcagcaggagaaggcccgcaggctggccagcctcgaactgcccgagcggcagcttagccgccaccggcgcacggcgctggacgagtaccgcagcgagatggccgggctggtcaagcgcttcggtgatgacctcagcaagtgcgactttatcgccgcgcagaagctggccagccggggccgcagtgccgaggaaatcggcaaggccatggccgaggccagcccagcgctggcagagcgcaagcccggccacgaagcggattacatcgagcgcaccgtcagcaaggtcatgggtctgcccagcgtccagcttgcgcgggccgagctggcacgggcaccggcaccccgccagcgaggcatggacaggggcgggccagatttcagcatgtagtgcttgcgttggtactcacgcctgttatactatgagtactcacgcacagaagggggttttatggaatacgaaaaaagcgcttcagggtcggtctacctgatcaaaagtgacaagggctattggttgcccggtggctttggttatacgtcaaacaaggccgaggctggccgcttttcagtcgctgatatggccagccttaaccttgacggctgcaccttgtccttgttccgcgaagacaagcctttcggccccggcaagtttctcggtgactgatatgaaagaccaaaaggacaagcagaccggcgacctgctggccagccctgacgctgtacgccaagcgcgatatgccgagcgcatgaaggccaaagggatgcgtcagcgcaagttctggctgaccgacgacgaatacgaggcgctgcgcgagtgcctggaagaactcagagcggcgcagggcgggggtagtgaccccgccagcgcctaaccaccaactgcctgcaaaggaggcaatcaatggctacccataagcctatcaatattctggaggcgttcgcagcagcgccgccaccgctggactacgttttgcccaacatggtggccggtacggtcggggcgctggtgtcgcccggtggtgccggtaaatccatgctggccctgcaactggccgcacagattgcaggcgggccggatctgctggaggtgggcgaactgcccaccggcccggtgatctacctgcccgccgaagacccgcccaccgccattcatcaccgcctgcacgcccttggggcgcacctcagcgccgaggaacggcaagccgtggctgacggcctgctgatccagccgctgatcggcagcctgcccaacatcatggccccggagtggttcgacggcctcaagcgcgccgccgagggccgccgcctgatggtgctggacacgctgcgccggttccacatcgaggaagaaaacgccagcggccccatggcccaggtcatcggtcgcatggaggccatcgccgccgataccgggtgctctatcgtgttcctgcaccatgccagcaagggcgcggccatgatgggcgcaggcgaccagcagcaggccagccggggcagctcggtactggtcgataacatccgctggcagtcctacctgtcgagcatgaccagcgccgaggccgaggaatggggtgtggacgacgaccagcgccggttcttcgtccgcttcggtgtgagcaaggccaactatggcgcaccgttcgctgatcggtggttcaggcggcatgacggcggggtgctcaagcccgccgtgctggagaggcagcgcaagagcaagggggtgccccgtggtgaagcctaagaacaagcacagcctcagccacgtccggcacgacccggcgcactgtctggcccccggcctgttccgtgccctcaagcggggcgagcgcaagcgcagcaagctggacgtgacgtatgactacggcgacggcaagcggatcgagttcagcggcccggagccgctgggcgctgatgatctgcgcatcctgcaagggctggtggccatggctgggcctaatggcctagtgcttggcccggaacccaagaccgaaggcggacggcagctccggctgttcctggaacccaagtgggaggccgtcaccgctgaatgccatgtggtcaaaggtagctatcgggcgctggcaaaggaaatcggggcagaggtcgatagtggtggggcgctcaagcacatacaggactgcatcgagcgcctttggaaggtatccatcatcgcccagaatggccgcaagcggcaggggtttcggctgctgtcggagtacgccagcgacgaggcggacgggcgcctgtacgtggccctgaaccccttgatcgcgcaggccgtcatgggtggcggccagcatgtgcgcatcagcatggacgaggtgcgggcgctggacagcgaaaccgcccgcctgctgcaccagcggctgtgtggctggatcgaccccggcaaaaccggcaaggcttccatagataccttgtgcggctatgtctggccgtcagaggccagtggttcgaccatgcgcaagcgccgccagcgggtgcgcgaggcgttgccggagctggtcgcgctgggctggacggtaaccgagttcgcggcgggcaagtacgacatcacccggcccaaggcggcaggctgaccccccccactctattgtaaacaagacatttttatcttttatattcaatggcttattttcctgctaattggtaataccatgaaaaataccatgctcagaaaaggcttaacaatattttgaaaaattgcctactgagcgctgccgcacagctccataggcc

SEQ ID NO: 26 - DNA, 1540 bp, Artificial Sequence
  Synthetic RCR
tccgccgccctagacctagtgtcattttatttcccccgtttcagcatcaagaacctttgcataacttgctctatatccacactgataattgccctcaaaccataatctaaaggcgctagagtttgttgaaacaatatcttttacatcattcgtatttaaaattccaaactccgctcccctaaggcgaataaaagccattaaatcttttgtatttaccaaattatagtcatccactatatctaagagtaaattcttcaattctcttttttggctttcatcaagtgttatatagcggtcaatatcaaaatcattaatgttcaaaatatcttttttgtcgtatatatgtttattcttagcaatagcgtcctttgattcatgagtcaaatattcatatgaacctttgatataatcaagtatctcaacatgagcaactgaactattccccaattttcgcttaatcttgttcctaacgctttctattgttacaggatttcgtgcaatatatataacgtgatagtgtggttttttatagtgctttccatttcgtataacatcactactattccatgtatctttatcttttttttcgtccatatcgtgtaaaggactgacagccatagatacgcccaaactctctaatttttccttccaatcattaggaattgagtcaggatataataaaaatccaaaatttctagctttagtatttttaatagccatgatataattaccttatcaaaaacaagtagcgaaaactcgtatccttctaaaaacgcgagctttcgcttattttttttgttctgattcctttcttgcatattcttctatagctaacgccgcaaccgcagattttgaaaaacctttttgtttcgccatatctgttaattttttatcttgctcttttgtcagagaaatcataactctttttttcgattctgaaatcaccatttaaaaaactccaatcaaataattttataaagttagtgtatcactttgtaatcataaaaacaacaataaagctacttaaatatagatttataaaaaacgttggcgaaaacgttggcgattcgttggcgattgaaaaaccccttaaacccttgagccagttgggatagagcgtttttggcacaaaaattggcactcggcacttaatggggggtcgtagtacggaagcaaaattcgcttcctttccccccatttttttccaaattccaaatttttttcaaaaattttccagcgctaccgctcggcaaaattgcaagcaatttttaaaatcaaacccatgagggaatttcattccctcatactcccttgagcctcctccaaccgaaatagaagggcgctgcgcttattatttcattcagtcatcggctttcataatctaacagacaacatcttcgctgcaaagccacgctacgctcaagggcttttacgctacgataacgcctgttttaacgattatgccgataactaaacgaaataaacgctaaaacgtctcagaaacgattttgagacgttttaataaaaaatcgcctagtgc

SEQ ID NO: 27 - DNA, 20 bp, Artificial Sequence
  Synthetic 16S forward
agagtttgatcatggctcag

SEQ ID NO: 28 - DNA, 20 bp, Artificial Sequence
  Synthetic 16S reverse
cggttaccttgttacgactt

SEQ ID NO: 29 - DNA, 19 bp, Artificial Sequence
  Synthetic GFP validation primer forward
atgcgtaaaggcgaagagc

SEQ ID NO: 30 - DNA, 27 bp, Artificial Sequence
  Synthetic GFP validation primer reverse
ttatttgtacagttcatccataccatg

SEQ ID NO: 31 - DNA, 24 bp, Artificial Sequence
  Synthetic Beta-lactamase validation primer forward
atgagtattcaacatttccgtgtc

SEQ ID NO: 32 - DNA, 24 bp, Artificial Sequence
  Synthetic Beta-lactamase validation primer reverse
ttaccaatgcttaatcagtgaggc

SEQ ID NO: 33 - DNA, 20 bp, Artificial Sequence
  Synthetic pGT-B backbone validation primer forward
ctgcgcaacccaagtgctac

SEQ ID NO: 34 - DNA, 23 bp, Artificial Sequence
  Synthetic pGT-B backbone validation primer reverse
cagtccagagaaatcggcattca

SEQ ID NO: 35 - DNA, 27 bp, Artificial Sequence
  Synthetic pGT-Ah backbone validation primer forward
atggaaaaaaaggaatttcgtgttttg

SEQ ID NO: 36 - DNA, 27 bp, Artificial Sequence
  Synthetic pGT-Ah backbone validation primer reverse
ttattcaacatagttcccttcaagagc

SEQ ID NO: 37 - DNA, 25 bp, Artificial Sequence
  Synthetic CarbR internal forward primer
ccgaagaacgttttccaatgatgag

SEQ ID NO: 38 - DNA, 20 bp, Artificial Sequence
  Synthetic GFP internal reverse primer
tgattgtctggcagcagaac

SEQ ID NO: 39 - DNA, 26 bp, Artificial Sequence
  Synthetic catP (chlor resistance) validation primer forward
gcaagtgttcaagaagttattaagtc

SEQ ID NO: 40 - DNA, 28 bp, Artificial Sequence
  Synthetic catP (chlor resistance) validation primer reverse
ttaactatttatcaattcctgcaattcg

SEQ ID NO: 41 - DNA, 25 bp, Artificial Sequence
  Synthetic tetQ (tet resistance) internal forward primer
tggaagaacgattttccgtaaaggt